(19) United States
(12) Patent Application Publication
(10) Pub. No.: US 2026/0013884 A1
(43) Pub. Date: Jan. 15, 2026

(54) MEDICAL DEVICE FOR REMOVING MATERIAL FROM A VESSEL

(71) Applicant: MicroVention, Inc., Aliso Viejo, CA (US)

(72) Inventor: Belete Degaga, Mission Viejo, CA (US)

(73) Assignee: MicroVention, Inc., Aliso Viejo, CA (US)

(21) Appl. No.: 19/266,046

(22) Filed: Jul. 10, 2025

Related U.S. Application Data
(60) Provisional application No. 63/669,654, filed on Jul. 10, 2024.

(51) Int. Cl. A61B 17/22; A61B 17/00

(52) U.S. Cl. CPC A61B 17/22; A61B 17/00234; A61B 2017/00238; A61B 2017/00305; A61B 2017/00557; A61B 2017/22065

(57) ABSTRACT

A medical device includes a pusher and a clot-engaging device coupled to the pusher. The clot-engaging device includes a plurality of bristles extending from a surface of the clot-engaging device. In one configuration, each bristle of the plurality of bristles includes a first end attached to the surface of the clot-engaging device. In one configuration the material composition of each bristle in the first set of bristles is polymer or polymer-based. In one configuration, at least a portion of a body of each bristle of the plurality of bristles extends linearly in a radial direction from a center of the clot-engaging device. In another configuration, at least one bristle of the plurality of bristles is pointing in a tangential direction away from the surface of the clot-engaging device.

### CROSS-REFERENCE TO RELATED APPLICATION

This application claims the benefit of U.S. Provisional Patent Application Ser. No. 63/669,654 entitled “MEDICAL DEVICE FOR REMOVING MATERIAL FROM A VESSEL” filed on Jul. 10, 2024, which is expressly incorporated by reference herein in its entirety.

### TECHNICAL FIELD

The present disclosure relates generally to a medical device, and more particularly, to a device for retrieving a foreign body inside a human, such as a blood clot from a blood vessel.

### Introduction

The buildup of thrombi in vasculature can lead to formation of blood clots. The formation of clots can result in restricted blood supply to downstream areas of the vasculature. When located in the neurovascular system, these clots can lead to stroke.

Recent technologies to remove clots utilize devices designed to hold and capture the clot, followed by withdrawal of the device to physically remove the captured clots from the body. Several of these devices may fail to capture the clot in its entirety, or may promote clot fragmentation which may allow thrombi to dislodge and accumulate at another site, thus continuing the risk of stroke. In addition, several of these devices may promote endothelial denudation due to high friction between the device and the vessel wall.

There is need for an obstruction removal device which reduces the likelihood of fragmented thrombi staying in the vasculature while maximizing the chance of mechanically capturing the clot, and limiting the risk of endothelial denudation.

### BRIEF SUMMARY

The following presents a simplified summary of one or more aspects in order to provide a basic understanding of such aspects. This summary is not an extensive overview of all contemplated aspects. This summary neither identifies key or critical elements of all aspects nor delineates the scope of any or all aspects. Its sole purpose is to present some concepts of one or more aspects in a simplified form as a prelude to the more detailed description that is presented later.

In one aspect of the disclosure, a medical device is provided. The medical device may include a pusher and a clot-engaging device coupled to the pusher. The clot-engaging device may include a plurality of bristles from a surface of the clot-engaging device.

In one aspect of the disclosure, a medical device is provided. The medical device may include a pusher; and a clot-engaging device coupled to the pusher. The clot-engaging device may include at least one semi-continuous protrusion or groove or at least one cavity on an outer surface or an inner surface of the clot-engaging device.

In one aspect of the disclosure, a medical device is provided. The medical device may include a pusher with a distal end having an elongated member; a plurality of bristles, where each bristle has a proximal bristle end distributed along the elongated member and a distal bristle tip not distributed along the elongated member, and where the plurality of bristles includes: a first set of bristles distributed along a first cross-sectional portion of the elongated member, and a second set of bristles distributed along a second cross-sectional portion of the elongated member; and where the elongated member has a first bristle-free region located between the first cross-sectional portion and the second cross-sectional portion of the elongated member.

In one aspect of the disclosure, a medical device is provided. The medical device may include a pusher with a distal end having an elongated member; a plurality of bristles, where each bristle has a proximal bristle end distributed along the elongated member and a distal bristle tip not distributed along the elongated member, and where the plurality of bristles includes: a first set of bristles distributed along a first longitudinal portion of the elongated member, and a second set of bristles distributed along a second longitudinal portion of the elongated member; and where the elongated member has a first bristle-free region located between the first longitudinal portion and the second longitudinal portion of the elongated member.

In one aspect of the disclosure, a medical device is provided. The medical device may include an elongated member having an outer surface, inner lumen, and a tube body therebetween, where the elongated member has at least one semi-continuous protrusion or groove along the outer surface or an inner surface of the elongated member with a depth that is less than a thickness of the elongated member.

In one aspect of the disclosure, a medical device is provided. The medical device may include an elongated member having an outer surface, inner lumen, and a tube body therebetween, where the elongated member has a plurality of cavities along the outer surface each having a depth that is less than a thickness of the elongated member.

In one aspect of the disclosure, a medical device is provided. The medical device may include an elongated member having an outer surface, inner lumen, and a tube body therebetween; and at least one helical protrusion or groove that spirals helically and longitudinally along the outer surface of the elongated member for one or more rotations.

In one aspect of the disclosure, a medical device is provided. The medical device may include a balloon catheter having a proximal region, a distal region, and an internal lumen therebetween; an inflatable balloon coupled to the distal region of the balloon catheter, where the inflatable balloon is in fluid communication with an inflation port at the proximal region of the balloon catheter via an inflation channel within the balloon catheter; and a plurality of bristles distributed along the inflatable balloon or a spring shaft located, at least in part, along an axial length of the balloon catheter that includes the inflatable balloon.

In one aspect of the disclosure, a medical device is provided. The medical device may include a pusher with a distal end having an elongated member; and a set of bristle means for capturing a foreign body and coupled to the elongated member.

In one aspect of the disclosure, a medical device is provided. The medical device may include a pusher with a distal end having an elongated member; and a set of cavity means or a set of semi-continuous protrusion or groove means for capturing a foreign body and coupled to the elongated member.

In one aspect of the disclosure, a medical device is provided. The medical device may include a pusher with a distal end having an elongated member; and at least one capturing means for capturing a foreign body and distributed along an inflatable means associated with the elongated member.

In one aspect of the disclosure, a method of removing a material from a blood vessel is provided. The method may include navigating a catheter into the blood vessel to a target site that is distal to the material, where the catheter includes: a pusher with a distal end having an elongated member, a plurality of bristles, where each bristle has a proximal bristle end coupled to the elongated member and a distal bristle tip not coupled to the elongated member, and where the plurality of bristles includes: a first set of bristles coupled to a first longitudinal portion of the elongated member, and a second set of bristles coupled to a second longitudinal portion of the elongated member; and where the elongated member has a first bristle-free region located between the first longitudinal portion and the second longitudinal portion of the elongated member; engaging the plurality of bristles with the material; retracting the catheter, the plurality of bristles, and the material in a proximal direction.

In one aspect of the disclosure, a method of removing a material from a blood vessel is provided. The method may include navigating a catheter into the blood vessel to a target site that is distal to the material, where the catheter includes: a pusher with a distal end having an elongated member, a plurality of bristles, where each bristle has a proximal bristle end coupled to the elongated member and a distal bristle tip not coupled to the elongated member, and where the plurality of bristles includes: a first set of bristles coupled to a first cross-sectional portion of the elongated member, a second set of bristles coupled to a second cross-sectional portion of the elongated member; and where the elongated member has a first bristle-free region located between the first cross-sectional portion and the second cross-sectional portion of the elongated member; engaging the plurality of bristles with the material; retracting the catheter, the plurality of bristles, and the material in a proximal direction.

In one aspect of the disclosure, a method of removing a material from a blood vessel is provided. The method may include navigating a balloon catheter into the blood vessel to a target site that is distal to the material, where the balloon catheter includes an inflatable balloon having an outer surface with a plurality of bristles; inflating the balloon; engaging the plurality of bristles with the material; retracting the balloon catheter, the plurality of bristles, and the material in a proximal direction.

To the accomplishment of the foregoing and related ends, the one or more aspects include the features hereinafter fully described and particularly pointed out in the claims. The following description and the drawings set forth in detail certain illustrative features of the one or more aspects. These features are indicative, however, of but a few of the various ways in which the principles of various aspects may be employed.

### BRIEF DESCRIPTION OF THE DRAWINGS

FIG. 1 is a diagram illustrating an example of a clot retrieval device having a plurality of bristles, in accordance with an example embodiment of the present disclosure.

FIG. 2 is a diagram illustrating another view of the example clot retrieval device having a plurality of bristles, in accordance with an example embodiment of the present disclosure.

FIG. 3 is a diagram illustrating a sideview (cross-sectional) of the example clot retrieval device having a plurality of bristles, in accordance with an example embodiment of the present disclosure.

FIG. 4 is a diagram illustrating a cross-sectional view of a cross-section of the example clot retrieval device having a plurality of bristles, in accordance with an example embodiment of the present disclosure.

FIG. 5A is a diagram illustrating a cross-sectional view of a cross-section of a different example clot retrieval device having a plurality of bristles distributed along an inner surface of an elongated member that is hollow, in accordance with an example embodiment of the present disclosure.

FIG. 5B is a diagram illustrating a cross-sectional view of a cross-section of a different example clot retrieval device having a plurality of bristles distributed along an inner surface and an outer surface of an elongated member that is hollow, in accordance with an example embodiment of the present disclosure.

FIG. 6 is a diagram illustrating an example of a clot retrieval device having a plurality of hook-shaped bristles, in accordance with an example embodiment of the present disclosure.

FIG. 7 is a diagram illustrating an example of a clot retrieval device having a plurality of hook-shaped bristles and non-hook-shaped bristles of different length, in accordance with an example embodiment of the present disclosure.

FIG. 8 is a diagram illustrating an example of a clot retrieval device having a plurality of bristles and a tapered elongated member, in accordance with an example embodiment of the present disclosure.

FIG. 9 is another view of a diagram illustrating an example of a clot retrieval device having a plurality of bristles and a tapered elongated member, in accordance with an example embodiment of the present disclosure.

FIG. 10 is another view of a diagram illustrating an example of a clot retrieval device having a plurality of bristles and a tapered elongated member, in accordance with an example embodiment of the present disclosure.

FIG. 11 is an example of a clot retrieval device having a plurality of bristles that may each be a straight bristle that is aligned in a radial direction extending outwardly from a center of the elongated member, in accordance with an example embodiment of the present disclosure.

FIG. 12 is a diagram illustrating an example of a clot retrieval device with various example types of bristles, in accordance with an example embodiment of the present disclosure.

FIG. 13 is a diagram illustrating an example of a clot retrieval device that includes an inflatable balloon, in accordance with an example embodiment of the present disclosure.

FIG. 14 is a diagram illustrating an example of a clot retrieval device that includes an inflatable balloon, in accordance with an example embodiment of the present disclosure.

FIG. 15 is a diagram illustrating an example of a clot retrieval device that includes an inflatable balloon and a plurality of bristles distributed along an outer surface of the inflatable balloon, in accordance with an example embodiment of the present disclosure.

FIG. 16 is a diagram illustrating an example of a clot retrieval device that includes an inflatable balloon and a plurality of bristles distributed along an outer surface of the inflatable balloon, in accordance with an example embodiment of the present disclosure.

FIG. 17 is a diagram illustrating an example of a clot retrieval device with at least one helical semi-continuous protrusion or groove and at least one cavity, in accordance with an example embodiment of the present disclosure.

FIGS. 18A-18B are diagrams illustrating two different sideviews, cross-sectional and non-cross-sectional, of the example clot-engaging device with at least one helical semi-continuous protrusion or groove and at least one cavity, in accordance with an example embodiment of the present disclosure.

FIG. 19A is a diagram illustrating a perspective view of the example clot-engaging device with at least one helical semi-continuous protrusion or groove and at least one cavity, in accordance with an example embodiment of the present disclosure.

FIG. 19B is a diagram illustrating a sideview for the example clot-engaging device with at least one helical semi-continuous protrusion or groove and at least one cavity, in accordance with an example embodiment of the present disclosure.

FIG. 20 is a diagram illustrating an example of a clot retrieval device with at least one helical semi-continuous protrusion or groove and at least one cavity, in accordance with an example embodiment of the present disclosure.

FIG. 21 is a diagram illustrating a particular segment of the example clot retrieval device with at least one helical semi-continuous protrusion or groove and at least one cavity, in accordance with an example embodiment of the present disclosure.

FIG. 22 is a diagram illustrating an example clot-engaging device with at least one helical semi-continuous protrusion or groove and at least one cavity, in accordance with an example embodiment of the present disclosure.

FIG. 23 is a diagram illustrating a side view of a section of the example clot-engaging device with at least one helical semi-continuous protrusion or groove and at least one cavity, in accordance with an example embodiment of the present disclosure.

FIG. 24 is a diagram illustrating a top view of the example clot-engaging device with at least one helical semi-continuous protrusion or groove and at least one cavity, in accordance with an example embodiment of the present disclosure.

FIG. 25 is a diagram illustrating an example of a clot retrieval device having a plurality of bristles and a plurality of coils, in accordance with an example embodiment of the present disclosure.

FIG. 26 is a diagram illustrating a sideview of an example of the clot retrieval device having a plurality of bristles and a plurality of coils, in accordance with an example embodiment of the present disclosure.

FIG. 27 is a diagram illustrating a sideview of an example of the clot retrieval device having a plurality of bristles and a plurality of coils, in accordance with an example embodiment of the present disclosure.

FIG. 28 is a diagram illustrating a sideview of an example of a clot retrieval device having a plurality of helical protrusions, in accordance with an example embodiment of the present disclosure.

FIG. 29 is a diagram illustrating a sideview (cross-sectional) of an example of a clot retrieval device having a plurality of helical protrusions, in accordance with an example embodiment of the present disclosure.

FIG. 30 is a diagram illustrating a sideview of an example of a clot retrieval device having a plurality of bristles of different length, in accordance with an example embodiment of the present disclosure.

FIG. 31 is a diagram illustrating a sideview (cross-sectional) of an example of a clot retrieval device having a plurality of bristles of different length, in accordance with an example embodiment of the present disclosure.

FIG. 32 is a flowchart of a method of removing a material, in accordance with an example embodiment of the present disclosure.

FIG. 33 is a flowchart of a method of removing a material, in accordance with an example embodiment of the present disclosure.

### DETAILED DESCRIPTION

Specific embodiments of the present disclosure will now be described with reference to the accompanying drawings. Aspects of the present disclosure may, however, be embodied in many different forms and should not be construed as limited to the embodiments set forth herein; rather, these embodiments are provided so that this disclosure will be thorough and complete, and will fully convey the scope of the present disclosure to those skilled in the art. The terminology used in the detailed description of the embodiments illustrated in the accompanying drawings is not intended to be limiting of the present disclosure. In the drawings, like numbers refer to like elements.

The detailed description set forth below in connection with the drawings describes various configurations and does not represent the only configurations in which the concepts described herein may be practiced. The detailed description includes specific details for the purpose of providing a thorough understanding of various concepts. However, these concepts may be practiced without these specific details.

Formation of thrombus in the vasculature can lead to clots over time, putting a patient at risk of ischemic stroke. Ischemic stroke caused by a fatty buildup may occur when plaque breaks off from an artery and travels to the brain. Clots may start in the heart and travel through the circulatory system. A clot may break up on its own or become lodged in an artery. When the clot blocks a brain artery, the brain may not obtain enough blood or oxygen, and cells may start to die. Approximately 87 percent of all strokes are ischemic strokes. Another type of major stroke is hemorrhagic stroke, in which a blood vessel in the brain ruptures and causes bleeding. The bleeding compresses brain tissue, damaging or killing it. A third type of stroke is transient ischemic attack (TIA), which may also be referred to as a mini-stroke. This type of stroke is caused by a temporary blockage or decreased blood flow to the brain. Symptoms may disappear on their own. Global ischemia, which is a more severe type of ischemic stroke, may occur when the flow of oxygen to the brain is greatly reduced or completely stopped.

A pulmonary embolism (PE) is one of the leading causes of cardiovascular mortality behind acute myocardial infarction and stroke and a leading cause of in-hospital mortality. As an example, up to 40% of patients are classified as intermediate risk (sub massive) PE, defined by the presence hemodynamic stability with significant right ventricular (RV) dysfunction and/or cardiac injury. A catheter-directed thrombectomy was shown to reduce early RV strain in patients with acute intermediate-risk PE. A catheter system may include a large aspiration guide catheter that is placed through the right heart into the pulmonary artery (PA) through which a device advances to disrupt and extract the thrombus while simultaneously aspirating and withdrawing the thrombus.

As used herein, the terms “blood clot,” “clot,” “target material,” or “foreign body” may refer to undesirable material in a blood vessel, such as a coronary artery, carotid artery, femoral artery, or other types of artery. Clots within vessels can be formed from blood cells, collagen, cholesterol, plaque, fat, calcified plaque, bubbles, arterial tissue, aggregates of proteins (e.g., fibrin), and/or other miscellaneous fragments or combinations thereof. Clots can lodge, for example, in narrow regions of blood vessels that feed the major organs, and therefore, cause loss of oxygen-rich blood flow to surrounding tissues resulting in localized cell death or micro-infarcts. Cerebral micro-infarcts are ischemic strokes that may lead to confusion, disturbance of speech, paralysis, visual disturbances, balance disturbances, and even death. In the heart, clots can cause myocardial infarcts, i.e., heart attacks. If the clots are left untreated, they can be potentially life threatening, and therefore, the presence of clots in blood vessels need immediate medical intervention.

Clots may be treated or eliminated by utilizing biologic intervention, surgical intervention, or a combination of the two. Biologic treatments involve the delivery of agents directly to the clots by a catheter to either dissolve or at least stabilize it until the body can eliminate it. However, the disadvantage of treating clots with biologic agents is that they may expose much of the body to the agents, potentially leading to life-threatening bleeding complications.

Alternately or additionally, mechanical means can be used to remove the clots from a patient's vessel. There are a variety of mechanical means to remove clots, including aspiration and mechanical thrombectomy. Mechanical treatments may involve aspiration, maceration, and compression of the clots within the vessel and finally removing it either by invasive surgery or by non-invasive means, such as with an aspiration catheter connected to an aspiration source (e.g., pump or syringe). The distinct advantage of invasive or non-invasive mechanical treatment is that it directly attacks the clots and eliminates the vascular obstruction without affecting non-diseased areas of the body, unlike biologic agents.

The technique of mechanical thrombectomy may involve utilizing a mechanical device to grip and withdraw the clot. These mechanical devices may be referred to as clot retrieval devices. Many clot retrieval or devices may have a distally open configuration to enable the devices to grip and remove clots from the vasculature.

Some of the traditional clot retrieval devices may be metal-based (e.g., made using metal). A metal-based clot retrieval device may be a long, thin, self-expanding mesh tube that is attached to a wire. The metal-based clot retrieval device may be placed within a catheter (which may be a larger tube), which allows an operator to position the metal-based clot retrieval device.

The heart undergoes changes with aging. Many changes are due to the development of heart disorders, which become more common with aging. Other changes are due to aging itself. As people age, the heart tends to enlarge slightly, developing thicker walls and slightly larger chambers. The increase in size is mainly due to an increase in the size of individual heart muscle cells. Thicker walls also become stiffer, which may not allow the chambers to fill with as much blood before each ventricle pumps. The age-related stiffening of the heart walls causes the left ventricle to not fill as well and may sometimes lead to heart failure (called diastolic heart failure or heart failure with preserved ejection fraction), especially in older people with other diseases such as high blood pressure, obesity, and diabetes.

The walls of the arteries and arterioles may become thicker, and the space within the arteries may expand slightly. Elastic tissue within the walls of the arteries and arterioles may be lost. Together, these changes may make the vessels stiffer and less elastic. Therefore, blood clot retrieval devices that are also stiffer and less elastic (e.g., metal-based or stiffer polymer-based) may be less compatible within such blood vessels and may increase the risk of inadvertently rupturing weaker blood vessels during a clot retrieval procedure.

Based on aspects provided herein, a clot retrieval device may be constructed from any appropriate materials, including but not limited to polymers, metals, other non-metals, or combinations thereof. For example, material composition of the clot retrieval device may be polymer-based (e.g., made using polymer without metal, made using polymer and metal, or include a polymer coating on an outer surface of the clot retrieval device). One advantage of various aspects provided herein may include being able to retrieve smaller clots and clean the blood vessel by including various bristles, cavities, protrusion, or groove. Another advantage of various aspects provided herein may include being gentle and soft inside the blood vessel for less harm and increased safety.

As used herein, the term “clot retrieval device” may be used to refer to a medical device for removing a foreign body, such as a blood clot, from a blood vessel. As used herein, the term “clot-engaging device” may be used to refer to a component on the clot retrieval device that may be configured to engage with the foreign body to remove the foreign body, such as a blood clot, from a blood vessel. The clot-engaging device may be an elongated member on the clot retrieval device. As used herein, the terms “clot-engaging device” and “elongated member” may be used interchangeably. A clot retrieval device may be included in a catheter and moved to a target site so that the clot-engaging device may engage with the foreign body.

The clot retrieval device may include a pusher with a distal end having an elongated member, which may be polymer-based (e.g., made using polymer without metal or include a polymer coating on an outer surface of the clot retrieval device) and may have a plurality of bristles distributed along an outer surface or an inner surface of the elongated member, which may also be polymer-based. In some aspects, alternatively or additionally, the clot retrieval device may include at least one cavity or at least one semi-continuous protrusion or groove (e.g., which may be constructed based on molding or sweep and cut) distributed along an outer surface or an inner surface of the elongated member. A sweep and cut may also be referred to as a “sweep cutout.” A sweep cutout may be constructed based on removing a portion on the surface (during construction) based on a pattern. In some aspects, each element of a clot retrieval device may be constructed using a same or different materials. In some aspects, the at least one semi-continuous protrusion or groove may be helical (e.g., spirals helically for one or more rotations) and may have a counterclockwise helical pattern (e.g., oriented in a counterclockwise direction) or a clockwise helical pattern (e.g., oriented in a clockwise direction). In some aspects, the clot-engaging device may be a detachable component of the clot-retrieval device that is configured to engage with the target material. In some aspects, the clot-engaging device may be constructed as a non-detachable part of the clot-retrieval device. In some aspects, the clot-engaging device may include and may be mounted on an inflatable balloon and there may be bristles or other components distributed along an outer surface of the inflatable balloon. In some aspects, there may be an intermediate layer between the bristles and the inflatable balloon. In some aspects, there may be a spring shaft in the inflatable balloon. The spring shaft may be a spring coil shaft used for loading the inflatable balloon (e.g., into a catheter).

The materials used to construct the clot retrieval device may be any material, such as polymer, metal, polymer and metal, or any other material that have a particular durometer within a defined suitable durometer range and a particular Shore hardness value within a defined suitable Shore hardness range. In some aspects, the suitable durometer range and the defined suitable Shore hardness range may be based on the particular type of blood vessel (e.g., brain, pulmonary, or the like). In some aspects, different elements of the clot retrieval device may be constructed using different materials and each element may have its respective suitable durometer range or suitable Shore hardness range. In some aspects, the clot retrieval device may be hollow. In some aspects, the clot retrieval device may be non-hollow. As an example, the clot retrieval device may be cylindrically shaped (e.g., conically shaped). In aspects where the clot retrieval device may be hollow, bristles, cavities, or grooves/protrusions may be distributed along an outer surface or an inner surface of the clot retrieval device. In aspects where the clot retrieval device may be non-hollow, bristles, cavities, or protrusions may be distributed along (e.g., disposed at) an outer surface of the clot retrieval device.

As used herein, the term “bristle” may refer to a fine and flexible filament that may be construed using a variety of materials, such as nylon, polyester, polypropylene, fiber, or other materials (such as other polymer-based materials or non-polymer materials). As used herein, the term “delivery configuration” may refer to a configuration of a clot retrieval device where the clot retrieval device is being moved towards the target material (e.g., foreign body, blood clot or blood clot fragment, material causing embolism, etc.) to a destination near the target material for the purpose of retrieving the target material. In such a configuration, the clot-engaging device may be in an “unconstrained configuration” where it could move inside a catheter (e.g., a tube). For example, an operator may move the clot retrieval device to a target location (e.g., proximate to the target material or distal to the target material) in a delivery configuration and the clot-engaging device may be moving in a catheter (e.g., a tube). In some aspects, in the delivery configuration, the clot retrieval device may be within a lumen of a catheter.

Once the clot retrieval device is moved to the target location, the clot retrieval device may be switched to a “deployed configuration” to engage with the target material. In some aspects, in the deployed configuration, the clot retrieval device is distal to the catheter (e.g., the clot retrieval device is no longer within a lumen of the catheter or a portion of the clot retrieval device is no longer within the lumen of the catheter). As used herein, the term “pusher” may refer to a component that enables the push/pull movement of the pusher (e.g., relative to the catheter) so that the clot retrieval device may switch between the delivery configuration and the deployed configuration. The pusher may be pulled to cause the clot retrieval device to be within the catheter (e.g., to switch to the delivery configuration) and may be pushed to cause the clot retrieval device to be distal from the catheter (e.g., to switch to the deployed configuration). Once the clot retrieval device is in the deployed configuration, the clot retrieval device may engage with the target material and the target material may be captured. The state where the target material is captured (e.g., trapped in a cavity or groove, captured by a bristle or protrusion, or otherwise captured) may be referred to as a “collection configuration.” After the target material is captured, the entire catheter may be pulled out along with the clot engaging device and the pusher so that the target material may be removed. When at least one bristle engages with the target material, the bristles may be in a “clot-engaging configuration.” When the clot retrieval device is in the delivery configuration and moving towards the target material, the bristles may be in a “resting configuration.” As used herein, the term “semi-continuous protrusion or groove” may refer to a structure, such as a raised structure or groove-like structure based on a semi-continuous or continuous pattern, such as a counterclockwise helix or a clockwise helix. A semi-continuous protrusion or groove may be constructed based on molding or sweep and cut of the clot retrieval device. In some aspects, the cavity or the semi-continuous protrusion or groove may have a depth that is less than the thickness of the elongated member. In some aspects, the semi-continuous protrusion or groove may be raised and may have a height (which may be a negative depth). There may be multiple overlapping or non-overlapping semi-continuous protrusions, semi-continuous grooves, or cavities on an elongated member of the clot retrieval device.

In some aspects, one or more bristles of the clot retrieval device may be distributed along (e.g., planted, ingrown, or otherwise coupled) an outer surface of the cylindrical polymeric tube of the clot retrieval device and arranged in a particular pattern. In some aspects, the bristles may be arranged circumferentially with multiple repeats axially. In some aspects, the bristles may be arranged axially with multiple repeats circumferentially. In some aspects, the bristles may have a rounded tip, which means that a distal end from a point where a bristle is coupled to the cylindrical polymeric tube of the clot retrieval device may be rounded. In some aspects, the bristles may be of a circular shape (cross-sectional shape, such as a cross-section from a top-down or down-top view of the bristle), a square shape, a hook shape, a rectangular shape, a polygon shape, a triangular shape, an oval shape, a trapezoidal shape, a straight line longitudinally, a zigzag line longitudinally, a bent line longitudinally, or any other shape. In some aspects, the bristles may be of a same length or different lengths. For example, in some aspects, each set of bristles over a cross-sectional portion of the cylindrical polymeric tube may have a respective length that may be the same as or different from the length(s) of other sets of bristles. In some aspects, each set of bristles over a longitudinal portion of the cylindrical polymeric tube may have a respective length that may be the same as or different from the length(s) of other sets of bristles. In some aspects, each bristle may have its own respective length that may be the same as or different from the length(s) of other bristles. In some aspects, the bristle length may be based on a blood vessel size. In other words, one may select a clot retrieval device having bristles of a suitable length based on the blood vessel size that contains the target material to be retrieved. In some aspects, the bristles may be polymer-based, soft, include a bend recovery property above a threshold, include a flex fatigue resistance above a threshold, include an abrasion resistance above a threshold, and/or include a wet stiffness resistance above a threshold. By having a good flex fatigue resistance (e.g., a flex fatigue resistance above a threshold), the bristle may be more likely to withstand repeated flexing or bending due to usage. By having a good abrasion resistance (e.g., an abrasion resistance above a threshold), the bristle may be able to resist more wear by rubbing or friction due to usage. By having a good bend recovery (e.g., a bend recovery above a threshold), the bristles may be more likely to return back to its original shape after it engages the target material. By having a good wet stiffness resistance (e.g., a wet stiffness resistance above a threshold), the bristles may be more likely to resist changes in shape when wet and being acted on by a physical force.

In some aspects, in addition to or alternative to bristles, the clot retrieval device may also include one or more coils. In some aspects, the coils may be placed in a coil region between the bristle regions (viewed axially along a height of the elongated member of the clot retrieval device, which may be cylindrical or conical). In some aspects, the clot retrieval device may include an inflatable balloon. In some aspects, the plurality of bristles may be coupled to the inflatable balloon by an intermediate layer, and the plurality of bristles may be coupled to an exterior surface of the intermediate layer. The inflatable balloon may be coupled to an interior surface of the intermediate layer. In a deflated configuration, the inflatable balloon may be deflated and the clot-retrieval device may not be engaging with the target. In the deflated configuration, an operator may move the clot-retrieval device (e.g., via the pusher) to the target and engage the target by inflating the balloon so that the balloon changes to an inflated configuration where the elongated member (e.g., bristles or other components on the elongated member) may engage with the target. In some aspects, on the elongated member that includes the inflatable balloon, there may be a spring shaft along an axial length of the elongated member, which may be a balloon catheter. In some aspects, the elongated member may be further included in a larger tube (e.g., a catheter) when the elongated member is moving towards a target site that includes the target material. In some aspects where the inflatable balloon is used, the elongated member or the entire clot-retrieval device including the elongated member may be referred to as a “balloon catheter.”

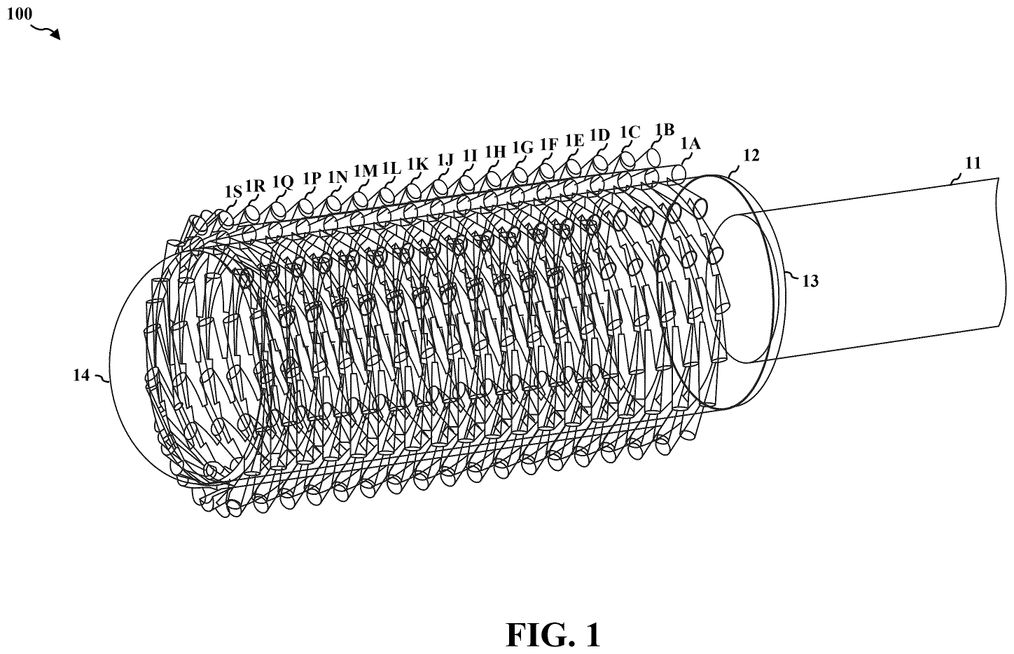

FIG. 1 is a diagram illustrating an example of a clot retrieval device 100 having a plurality of bristles 1A-1S. As shown in FIG. 1, a clot retrieval device 100 may include a pusher 11 (partially shown) and an elongated member 12 having a proximal end 13 (e.g., non-distal end) and a distal end 14. In some aspects, the distal end 14 may be rounded (e.g., dome-shaped). In some aspects, the elongated member 12 may be referred to as a clot-engaging device. In some aspects, the elongated member 12 may be a polymer-based elongated member which may be made using polymer without metal, made using polymer and metal, or include a polymer coating on an outer surface of the elongated member 12. The elongated member 12 may have a plurality of bristles 1A-1S coupled to it. In the example shown in FIG. 1, the plurality of bristles 1A-1S may be each pointing in a tangential direction that corresponds to a respective tangent line along the outer surface of the elongated member 12. As described herein, all the example elongated member(s) shown may be polymer-based elongated member which may be made using polymer without metal, made using polymer and metal, or include a polymer coating on an outer surface of the elongated member.

As illustrated in FIG. 1, a first set of bristles along a first cross-sectional portion of the elongated member 12 may include the bristle 1A, a second set of bristles along a second cross-sectional portion of the elongated member 12 may include the bristle 1B, a third set of bristles along a third cross-sectional portion of the elongated member 12 may include the bristle IC, a fourth set of bristles along a fourth cross-sectional portion of the elongated member 12 may include the bristle 1D, a fifth set of bristles along a fifth cross-sectional portion of the elongated member 12 may include the bristle 1E, a sixth set of bristles along a sixth cross-sectional portion of the elongated member 12 may include the bristle IF, a seventh set of bristles along a seventh cross-sectional portion of the elongated member 12 may include the bristle 1G, an eighth set of bristles along an eighth cross-sectional portion of the elongated member 12 may include the bristle 1H, a ninth set of bristles along a ninth cross-sectional portion of the elongated member 12 may include the bristle 1I, a tenth set of bristles along a tenth cross-sectional portion of the elongated member 12 may include the bristle 1J, an eleventh set of bristles along an eleventh cross-sectional portion of the elongated member 12 may include the bristle 1K, a twelfth set of bristles along a twelfth cross-sectional portion of the elongated member 12 may include the bristle IL, a thirteenth set of bristles along a thirteenth cross-sectional portion of the elongated member 12 may include the bristle 1M, a fourteenth set of bristles along a fourteenth cross-sectional portion of the elongated member 12 may include the bristle IN, a fifteenth set of bristles along a fifteenth cross-sectional portion of the elongated member 12 may include the bristle 1P (bristle label “10” was intentionally skipped to avoid ambiguity with number “10”), a sixteenth set of bristles along a sixteenth cross-sectional portion of the elongated member 12 may include the bristle IQ, a seventeenth set of bristles along a seventeenth cross-sectional portion of the elongated member 12 may include the bristle 1R, and an eighteenth set of bristles along an eighteenth cross-sectional portion of the elongated member 12 may include the bristle 1S. As shown in FIG. 1, the first set of bristles may be distributed along the circumference of the elongated member based on a pattern. For example, the pattern may be based on the first set of bristles including the bristle 1A being associated with a same axial distance associated with the elongated member 12.

In some aspects, each set of bristles along each respective cross-sectional portion of the elongated member 12 may be parallel with each other. In some aspects, alternatively, one can view the bristle 1A, the bristle 1B, . . . , and the bristle 1S as a set of bristles along a longitudinal portion of the elongated member 12. In some aspects, the entire clot retrieval device shown in FIG. 1 may be within a lumen of a catheter in a delivery configuration and the clot retrieval device may be distal to the catheter in a deployed configuration.

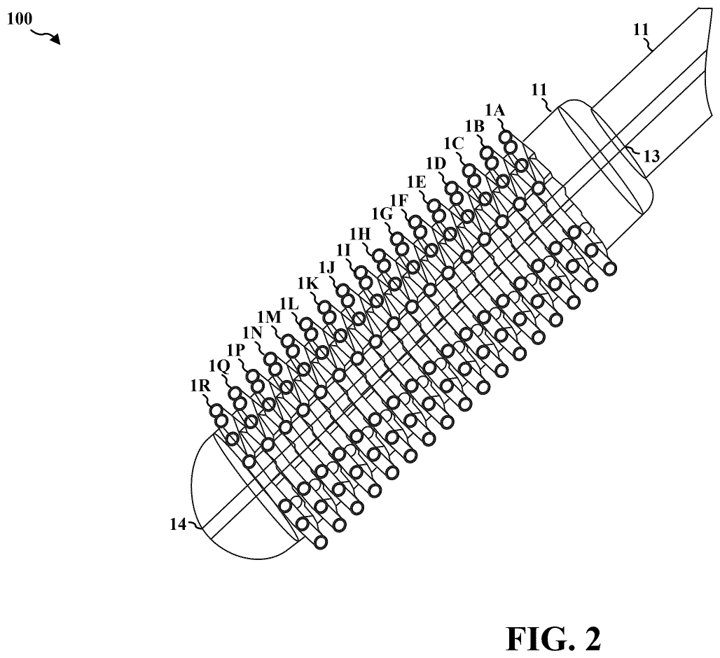

FIG. 2 is a diagram illustrating another view of the example clot retrieval device 100 having a plurality of bristles 1A-1R. As shown in FIG. 2, a clot retrieval device may include a pusher 11 (partially shown) and an elongated member 12 having a proximal end 13 (e.g., non-distal end) and a distal end 14. In some aspects, the elongated member 12 may be a polymer-based elongated member which may be made using polymer without metal or include a polymer coating on an outer surface of the elongated member 12. The elongated member 12 may have a plurality of bristles coupled to it. In the example shown in FIG. 2, the plurality of bristles 1A-1R may be each pointing in a tangential direction away from a circle associated with the elongated member 12.

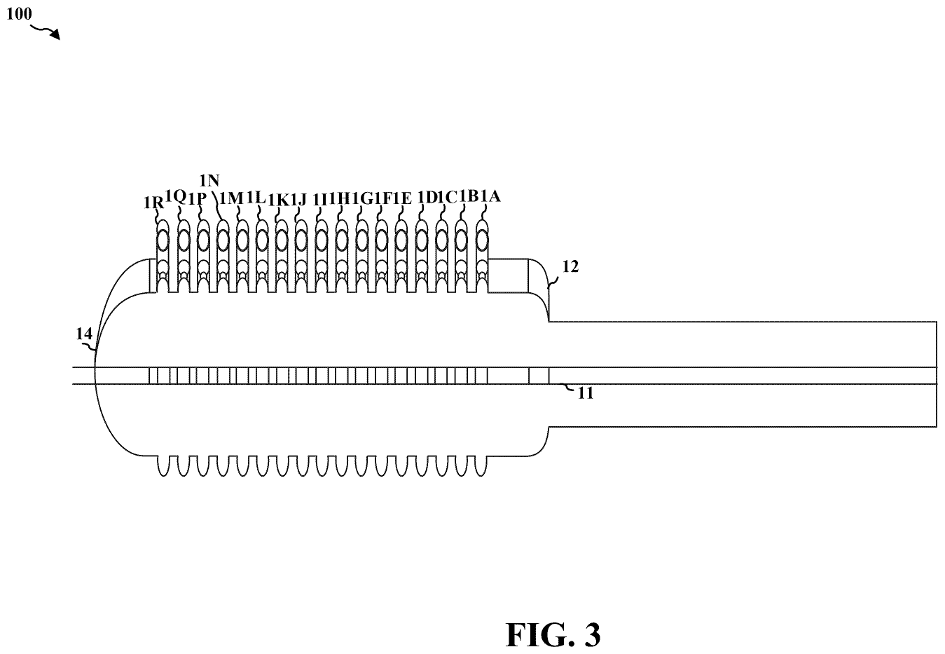

FIG. 3 is a diagram illustrating a sideview (cross-sectional cut 3-3 of FIG. 2) of the example clot retrieval device 100 having a plurality of bristles 1A-1S in FIGS. 1 and 1A-1R in FIG. 2. As shown in FIG. 3, a clot retrieval device may include a pusher 11 (partially shown) and an elongated member 12 having a proximal end 13 (e.g., non-distal end) and a distal end 34. In some aspects, the elongated member 12 may be a polymer-based elongated member which may be made using polymer without metal or include a polymer coating on an outer surface of the elongated member 12. The elongated member 12 may have a plurality of bristles coupled to it. In the example shown in FIG. 3, each of the plurality of bristles may be pointing in a tangential direction away from a circle associated with the elongated member 12.

In some aspects, each set of bristles along each cross-sectional portion of the elongated member 12 may be parallel with each other. In some aspects, the entire clot retrieval device shown in FIG. 3 may be inside of a lumen of a catheter in a delivery configuration and the clot retrieval device may be distal to (e.g., outside of) the catheter in a deployed configuration. The example in FIG. 1, FIG. 2, and FIG. 3 may be for exemplary purposes. More or less bristles may be utilized, and more or less bristles along each respective set of bristles may be utilized for the clot retrieval device. Bristles may be based on various body shapes or tip shapes. In some aspects, the body shape of each bristle may be one of straight, zigzagged, or bent. In some aspects, the tip shape of each bristle may be one of rounded, circular, square, rectangular, polygonal, triangular, helical, oval, or trapezoidal. As an example, a bristle may be hook-shaped (e.g., with a curved distal bristle tip). A bristle may also be extending from different directions. For example, a bristle may extend linearly in a radial direction from or toward a center of the elongated member or extend tangentially. In some aspects, one or more bristles coupled to the elongated member of the clot retrieval device may be hook-shaped, with a tip of the bristle pointing toward a distal end of the elongated member, a proximal end of the elongated member, or a direction perpendicular to the longitude of the elongated member. In some aspects, bristles may have different length or different width.

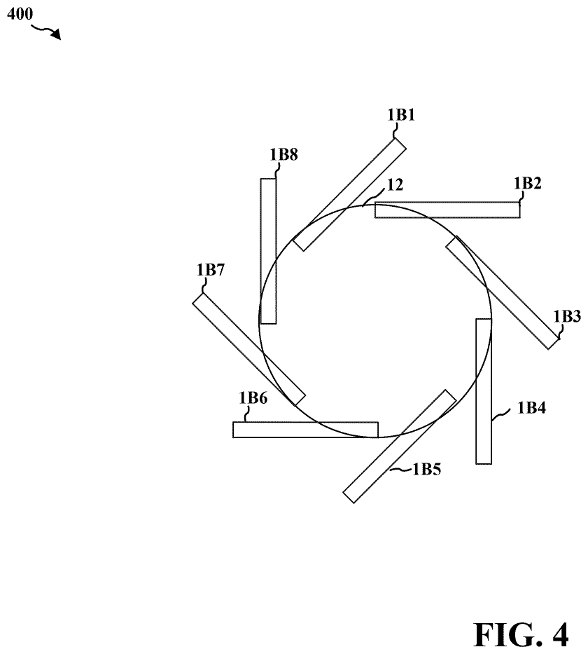

FIG. 4 is a diagram illustrating a cross-sectional view of an example cross-section (cross-sectional cut 4-4 of FIG. 2) of the example clot retrieval device having a plurality of bristles distributed along (e.g., attached to) an outer surface. As illustrated in FIG. 4, for a particular cross-section 400, which may be the second set of bristles distributed along a second cross-sectional portion of the elongated member 12 that may include the bristle 1B. There may be a number of different bristles including a first bristle 1B1, a second bristle 1B2, a third bristle 1B3, a fourth bristle 1B4, a fifth bristle 1B5, a sixth bristle 1B6, a seventh bristle 1B7, and an eighth bristle 1B8. Each of the first bristle 1B1, the second bristle 1B2, the third bristle 1B3, the fourth bristle 1 B4, the fifth bristle 1B5, the sixth bristle 1B6, the seventh bristle 1B7, and the eighth bristle 1B8, may point in a different respective tangential direction away from the outer surface of the elongated member. The example in FIG. 4 may be for exemplary purposes. More or less bristles may be utilized, and more or less bristles along each respective set of bristles may be utilized for the clot retrieval device.

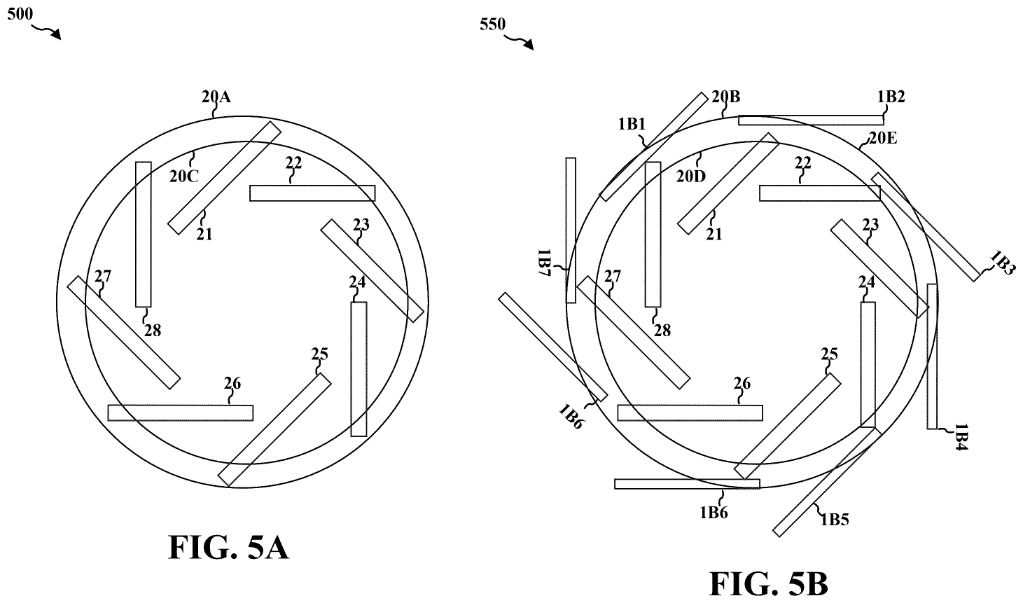

FIG. 5A is a diagram illustrating a cross-sectional view of a cross-section of a different example clot retrieval device having a plurality of bristles distributed along (e.g., attached to) an inner surface 20C of an example elongated member 20A that is hollow. As illustrated in FIG. 5A, for a particular cross-section 500, which may be a set of bristles distributed along a cross-sectional portion of the elongated member that may be hollow, there may be a number of different bristles including a first bristle 21, a second bristle 22, a third bristle 23, a fourth bristle 24, a fifth bristle 25, a sixth bristle 26, a seventh bristle 27, and an eighth bristle 28. Each of the first bristle 21, the second bristle 22, the third bristle 23, the fourth bristle 24, the fifth bristle 25, the sixth bristle 26, the seventh bristle 27, and the eighth bristle 28, may point in a different respective tangential direction away from an inner surface 20C of the hollow elongated member.

FIG. 5B is a diagram illustrating a cross-sectional view of a cross-section of a different example clot retrieval device having a plurality of bristles distributed along (e.g., attached to) an inner surface 20D and a plurality of bristles distributed along (e.g., attached to) an outer surface 20E of an example elongated member 20B that is hollow. As illustrated in FIG. 5B, for a particular cross-section 550, which may be a set of bristles distributed along a cross-sectional portion of the elongated member that may be hollow, there may be a number of different bristles including a first bristle 21, a second bristle 22, a third bristle 23, a fourth bristle 24, a fifth bristle 25, a sixth bristle 26, a seventh bristle 27, and an eighth bristle 28. Each of the first bristle 21, the second bristle 22, the third bristle 23, the fourth bristle 24, the fifth bristle 25, the sixth bristle 26, the seventh bristle 27, and the eighth bristle 28, may point in a different respective tangential direction away from the inner surface 20D of the hollow elongated member. In some embodiments, there may be a number of different bristles distributed along the outer surface 20E including a first bristle 1B1, a second bristle 1B2, a third bristle 1B3, a fourth bristle 1B4, a fifth bristle 1B5, a sixth bristle 1B6, a seventh bristle 1B7, and an eighth bristle 1B8. Each of the first bristle 1B1, the second bristle 1B2, the third bristle 1B3, the fourth bristle 1B4, the fifth bristle 1B5, the sixth bristle 1B6, the seventh bristle 1B7, and the eighth bristle 1B8, may point in a different respective tangential direction away from the outer surface 20E of the elongated member.

The example in FIG. 5A and FIG. 5B may be for exemplary purposes. More or less bristles may be used and bristles of various directions or shapes may be utilized for the clot retrieval device. Bristles may be based on various body shapes or tip shapes. In some aspects, the body shape of each bristle may be one of straight, zigzagged, or bent. In some aspects, the tip shape of each bristle may be one of rounded, circular, square, rectangular, polygonal, triangular, helical, oval, or trapezoidal. As an example, a bristle may be hook-shaped (e.g., with a curved distal bristle tip). A bristle may also be extending from different directions. For example, a bristle may extend linearly in a radial direction from or toward a center of the elongated member or extend tangentially. In some aspects, bristles may have different length or different width.

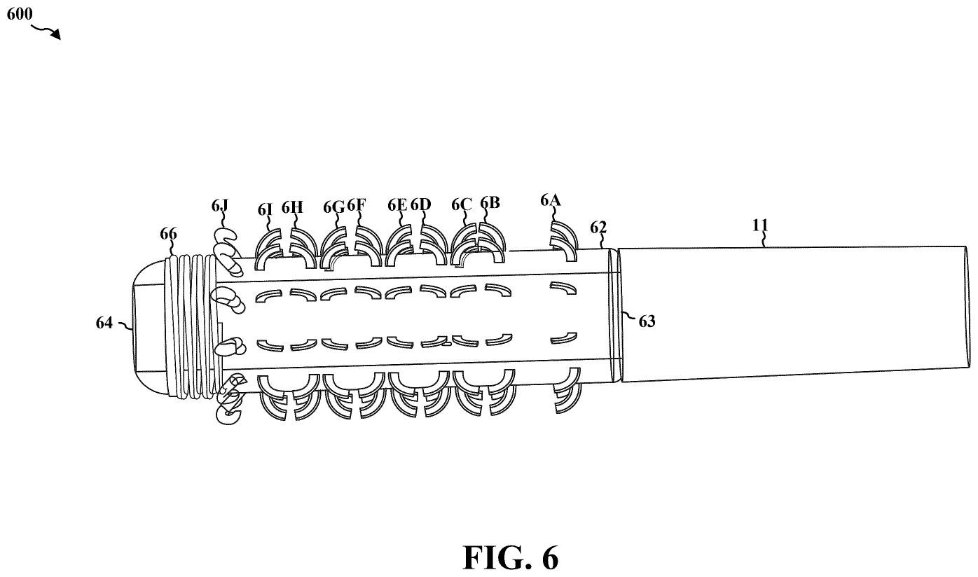

FIG. 6 is a diagram illustrating an example of a clot retrieval device 600 having a plurality of hook-shaped bristles 6A-6J (only hook-shaped bristles are shown in FIG. 6). As shown in FIG. 6, a clot retrieval device 600 may include a pusher 11 (partially shown) and an elongated member 62 having a proximal end 63 (e.g., non-distal end) and a distal end 64. In some aspects, the elongated member 62 may be referred to as a clot-engaging device. In some aspects, the elongated member 62 may be a polymer-based elongated member which may be made using metal, a polymer without metal, a polymer with metal, or include a polymer coating on an outer surface of the elongated member 62. The elongated member 62 may have a plurality of bristles coupled to it. In the example shown in FIG. 6, the plurality of bristles may be hooked bristles and may include a curvature shape, such as a curvature greater than 30 degrees, 60 degrees, 90 degrees, 135 degrees, 150 degrees, or the like.

As illustrated in FIG. 6, a first set of bristles along a first cross-sectional portion of the elongated member 62 may include the bristle 6A (which may be pointing to (face) the distal end 64), a second set of bristles along a second cross-sectional portion of the elongated member 62 may include the bristle 6B (which may be pointing to the distal end 64), a third set of bristles along a third cross-sectional portion of the elongated member 62 may include the bristle 6C (which may be pointing to the proximal end 63), a fourth set of bristles along a fourth cross-sectional portion of the elongated member 62 may include the bristle 6D (which may be pointing to the distal end 64), a fifth set of bristles along a fifth cross-sectional portion of the elongated member 62 may include the bristle 6E (which may be pointing to the proximal end 63), a sixth set of bristles along a sixth cross-sectional portion of the elongated member 62 may include the bristle 6F (which may be pointing to the distal end 64), a seventh set of bristles along a seventh cross-sectional portion of the elongated member 62 may include the bristle 6G (which may be pointing to the proximal end 63), an eighth set of bristles along an eighth cross-sectional portion of the elongated member 62 may include the bristle 6H (which may be pointing to the distal end 64), a ninth set of bristles along a ninth cross-sectional portion of the elongated member 62 may include the bristle 6I (which may be pointing to the proximal end 63), a tenth set of bristles along a tenth cross-sectional portion of the elongated member 62 may include the bristle 6J (which may be pointing to the proximal end 63).

In some embodiments, the elongated member 62 may include a radio-opaque marker 66. In some aspects, the radio-opaque marker 66 can be a band, wire, or coil and may be positioned around a distal end or portion of the elongated member 62 to aid in visualizing the distal end of the elongated member 62 during use. In some aspects, the radio-opaque marker 66 may be located on the pusher. In some aspects, the bristles 6A-6J may point in the same direction or in different combination of directions that are not illustrated in FIG. 6. In some embodiments, more or less bristles may be utilized, and more or less bristles along each respective set of bristles may be utilized for the clot retrieval device 600. In some aspects, the bristle 6B and the bristle 6C may be connected and may form one single loop-shaped bristle. Similarly, the bristle 6D and the bristle 6E, the bristle 6F and the bristle 6G, the bristle 6H and the bristle 6I, and other bristles not shown, may be connected with each other and may form respective loop-shaped bristles. In other words, in some aspects, the bristle 6B and the bristle 6C may be a first end and a second end of a single bristle. The first end and the second end may be both attached to the outer surface of the elongated member 62 and form a loop with the outer surface of the elongated member 62. The example in FIG. 6 may be for exemplary purposes. More or less bristles may be used and bristles of various directions or shapes may be utilized for the clot retrieval device.

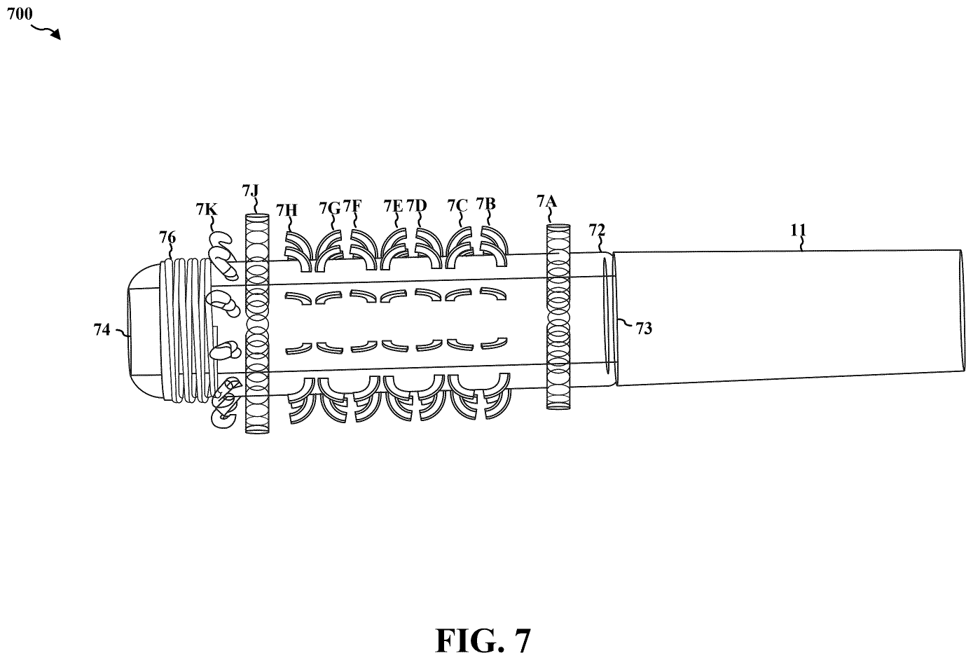

FIG. 7 illustrates an example of a clot retrieval device 700 having a plurality of hook-shaped bristles 7B-7H and 7K and non-hook-shaped bristles 7A, 7J of different length. As shown in FIG. 7, a clot retrieval device 700 may include a pusher 11 (partially shown) and an elongated member 72 having a proximal end 73 (e.g., non-distal end) and a distal end 74. In some aspects, the elongated member 72 may be referred to as a clot-engaging device. In some aspects, the elongated member 72 may be a polymer-based elongated member which may be made using polymer without metal or include a polymer coating on an outer surface of the elongated member 72. The elongated member 72 may have a plurality of bristles coupled to it. In the example shown in FIG. 7, the plurality of bristles may be hook-shaped bristles, or straight bristles pointing outwardly from a center of the elongated member 72. As illustrated in FIG. 7, a first set of bristles (that may be straight) along a first cross-sectional portion of the elongated member 72 may include the bristle 7A (which may be pointing outwardly from a center of the elongated member and may be of a first length), a second set of bristles (that may be hooked) along a second cross-sectional portion of the elongated member 72 may include the bristle 7B (which may be pointing to the distal end 74), a third set of bristles (that may be hooked) along a third cross-sectional portion of the elongated member 72 may include the bristle 7C (which may be pointing to the proximal end 54), a fourth set of bristles (that may be hooked) along a fourth cross-sectional portion of the elongated member 72 may include the bristle 7D (which may be pointing to the distal end 74), a fifth set of bristles (that may be hooked) along a fifth cross-sectional portion of the elongated member 72 may include the bristle 7E (which may be pointing to the proximal end 54), a sixth set of bristles (that may be hooked) along a sixth cross-sectional portion of the elongated member 72 may include the bristle 7F (which may be pointing to the distal end 74), a seventh set of bristles (that may be hooked) along a seventh cross-sectional portion of the elongated member 72 may include the bristle 7G (which may be pointing to the proximal end 54), an eighth set of bristles (that may be hooked) along an eighth cross-sectional portion of the elongated member 72 may include the bristle 7H (which may be pointing to the distal end 74), a ninth set of bristles (that may be hooked) along a ninth cross-sectional portion of the elongated member 72 may include the bristle 7I (which may be pointing to the proximal end 54), a tenth set of bristles (that may be straight) along a tenth cross-sectional portion of the elongated member 72 may include the bristle 7J (which may be pointing outwardly from a center of the elongated member and may be of a second length that is longer than the first length). Also, the elongated member 72 may include a radio-opaque marker 76 similar to the radio-opaque marker 66 described above with reference to FIG. 6. The example in FIG. 7 may be for exemplary purposes. More or less bristles may be used and bristles of various directions or shapes may be utilized for the clot retrieval device. Bristles may be based on various body shapes or tip shapes. In some aspects, the body shape of each bristle may be one of straight, zigzagged, or bent. In some aspects, the tip shape of each bristle may be one of rounded, circular, square, rectangular, polygonal, triangular, helical, oval, or trapezoidal. As an example, a bristle may be hook-shaped (e.g., with a curved distal bristle tip). A bristle may also be extending from different directions. For example, a bristle may extend linearly in a radial direction from or toward a center of the elongated member or extend tangentially. In some aspects, bristles may have different length or different width.

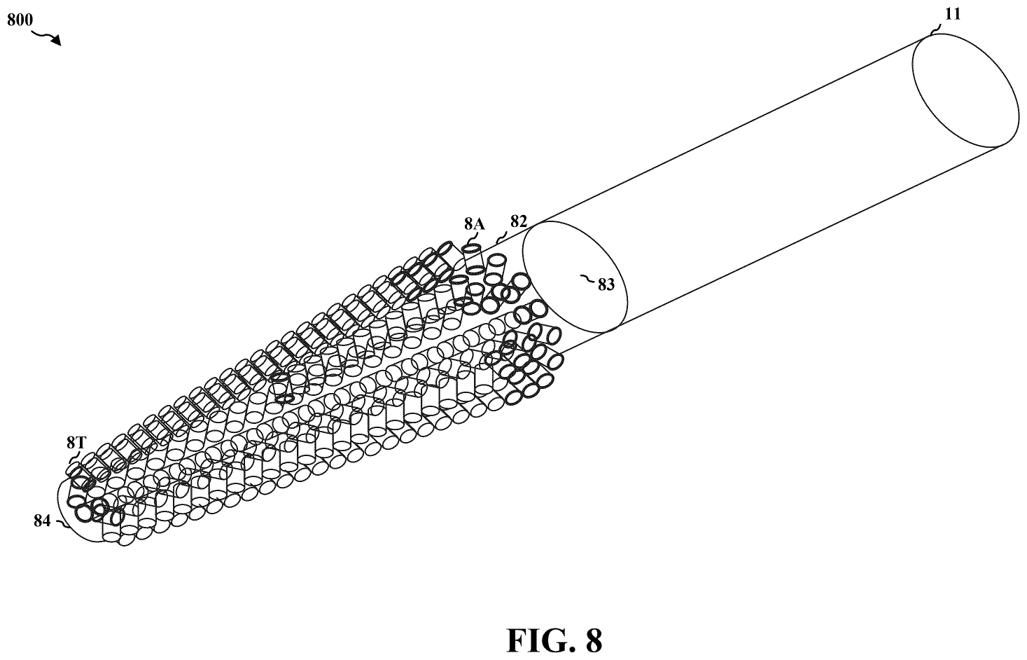

In some aspects, the clot retrieval device may have a tapered elongated member. FIG. 8 is a diagram illustrating an example of a clot retrieval device 800 having a plurality of bristles 8A-8T and a tapered elongated member 82. FIG. 8 is a diagram 8 illustrating an example of a clot retrieval device 800 having a plurality of bristles that may each be a straight bristle that is aligned in a radial direction extending outwardly from a center of the elongated member. As shown in FIG. 8, a clot retrieval device may include a pusher 81 (partially shown) and an elongated member 82 having a proximal end 83 (e.g., non-distal end) and a distal end 84. In some aspects, the elongated member 82 may be referred to as a clot-engaging device. In some aspects, the elongated member 82 may be a polymer-based elongated member which may be made using polymer without metal or include a polymer coating on an outer surface of the elongated member 82. The elongated member 82 may have a plurality of bristles coupled to it. In the example shown in FIG. 8, the plurality of bristles may be straight bristles pointing outwardly from a center of the elongated member 82. As shown in FIG. 8, the elongated member 82 may be tapered such that a first diameter of a first circle near the distal end 84 associated with the elongated member 82 is smaller than a second diameter of a second circle near the proximal end 83. In some aspects, the bristles may be shorter near the distal end 84 and may be longer near the proximal end 83. In some embodiments, the bristles near the distal end 84 may have the same length as the bristles near the proximal end 83. The example in FIG. 8 may be for exemplary purposes. More or less bristles may be used and bristles of various directions or shapes may be utilized for the clot retrieval device.

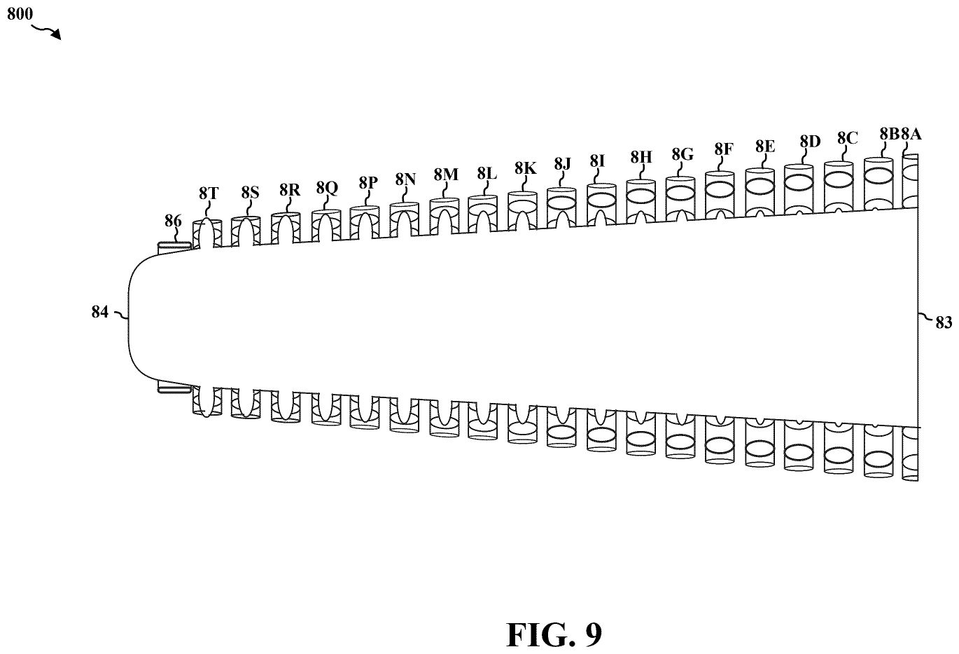

FIG. 9 is another view of a diagram illustrating a cross-sectional cut view of an example clot retrieval device 800 in FIG. 8 having a plurality of bristles and a tapered elongated member. The clot retrieval device in FIG. 9 may correspond to the tapered elongated member 82. As illustrated in FIG. 9, the tapered elongated member 82 may include a distal end 84 and a proximal end 83. As shown in FIG. 8, the elongated member 82 may be tapered such that a first diameter of a first circle near the distal end 84 associated with the elongated member 82 is smaller than a second diameter of a second circle near the proximal end 83. In some embodiments, the bristles may be shorter near the distal end 84 and may be longer near the proximal end 83.

As illustrated in FIG. 9, a first set of bristles along a first cross-sectional portion of the elongated member 82 may include the bristle 8A, a second set of bristles (e.g., which may have a length less than or equal to the length of the first set of bristles) along a second cross-sectional portion of the elongated member 82 may include the bristle 8B, a third set of bristles (e.g., which may have a length less than or equal to the length of the second set of bristles) along a third cross-sectional portion of the elongated member 82 may include the bristle 8C, a fourth set of bristles (e.g., which may have a length less than or equal to the length of the third set of bristles) along a fourth cross-sectional portion of the elongated member 82 may include the bristle 8D, a fifth set of bristles (e.g., which may have a length less than or equal to the length of the fourth set of bristles) along a fifth cross-sectional portion of the elongated member 82 may include the bristle 8E, a sixth set of bristles (e.g., which may have a length less than or equal to the length of the fifth set of bristles) along a sixth cross-sectional portion of the elongated member 82 may include the bristle 8F, a seventh set of bristles (e.g., which may have a length less than or equal to the length of the sixth set of bristles) along a seventh cross-sectional portion of the elongated member 82 may include the bristle 8G, an eighth set of bristles (e.g., which may have a length less than or equal to the length of the seventh set of bristles) along an eighth cross-sectional portion of the elongated member 82 may include the bristle 8H, a ninth set of bristles (e.g., which may have a length less than or equal to the length of the eighth set of bristles) along a ninth cross-sectional portion of the elongated member 82 may include the bristle 8I, a tenth set of bristles (e.g., which may have a length less than or equal to the length of the ninth set of bristles) along a tenth cross-sectional portion of the elongated member 82 may include the bristle 8J, an eleventh set of bristles (e.g., which may have a length less than or equal to the length of the tenth set of bristles) along an eleventh cross-sectional portion of the elongated member 82 may include the bristle 8K, a twelfth set of bristles (e.g., which may have a length less than or equal to the length of the eleventh set of bristles) along a twelfth cross-sectional portion of the elongated member 82 may include the bristle 8L, a thirteenth set of bristles (e.g., which may have a length less than or equal to the length of the twelfth set of bristles) along a thirteenth cross-sectional portion of the elongated member 82 may include the bristle 8M, a fourteenth set of bristles (e.g., which may have a length less than or equal to the length of the thirteenth set of bristles) along a fourteenth cross-sectional portion of the elongated member 82 may include the bristle 8N, a fifteenth set of bristles (e.g., which may have a length less than or equal to the length of the fourteenth set of bristles) along a fifteenth cross-sectional portion of the elongated member 82 may include the bristle 8P, a sixteenth set of bristles (e.g., which may have a length less than or equal to the length of the fifteenth set of bristles) along a sixteenth cross-sectional portion of the elongated member 82 may include the bristle 8Q, a seventeenth set of bristles (e.g., which may have a length less than or equal to the length of the sixteenth set of bristles) along a seventeenth cross-sectional portion of the elongated member 82 may include the bristle 8R, an eighteenth set of bristles (e.g., which may have a length less than or equal to the length of the seventeenth set of bristles) along an eighteenth cross-sectional portion of the elongated member 82 may include the bristle 8S, a nineteenth set of bristles (e.g., which may have a length less than or equal to the length of the eighteenth set of bristles) along a nineteenth cross-sectional portion of the elongated member 82 may include the bristle 8T, and so on. In some aspects, each set of bristles along each cross-sectional portion of the elongated member 82 may be parallel with each other. In some aspects, the entire clot retrieval device shown in FIG. 9 may be within a lumen of a catheter in a delivery configuration and the clot retrieval device may be distal to the catheter in a deployed configuration. Also, the elongated member 82 may include a radio-opaque marker 86.

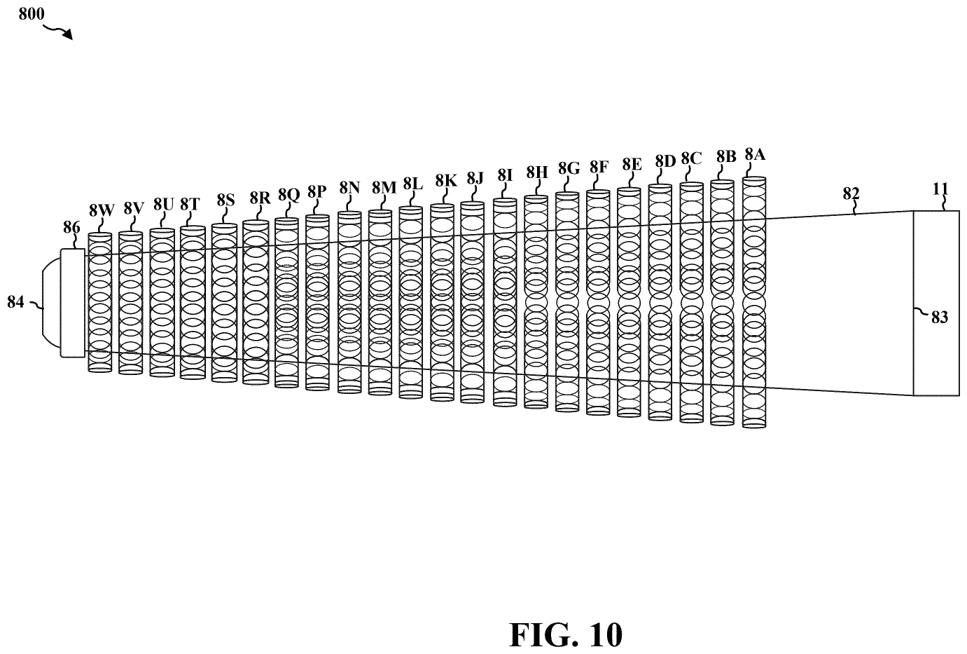

FIG. 10 is another view of a diagram illustrating an example sideview of the clot retrieval device 800 having a plurality of bristles 8A-8W and a tapered elongated member 82. Compared to the example in FIG. 9, the example in FIG. 10 may include additional sets of bristles that include bristle 8U, bristle 8V, and bristle 8W. The example in FIG. 9 and FIG. 10 may be for exemplary purposes. More or less bristles may be used and bristles of various directions or shapes may be utilized for the clot retrieval device.

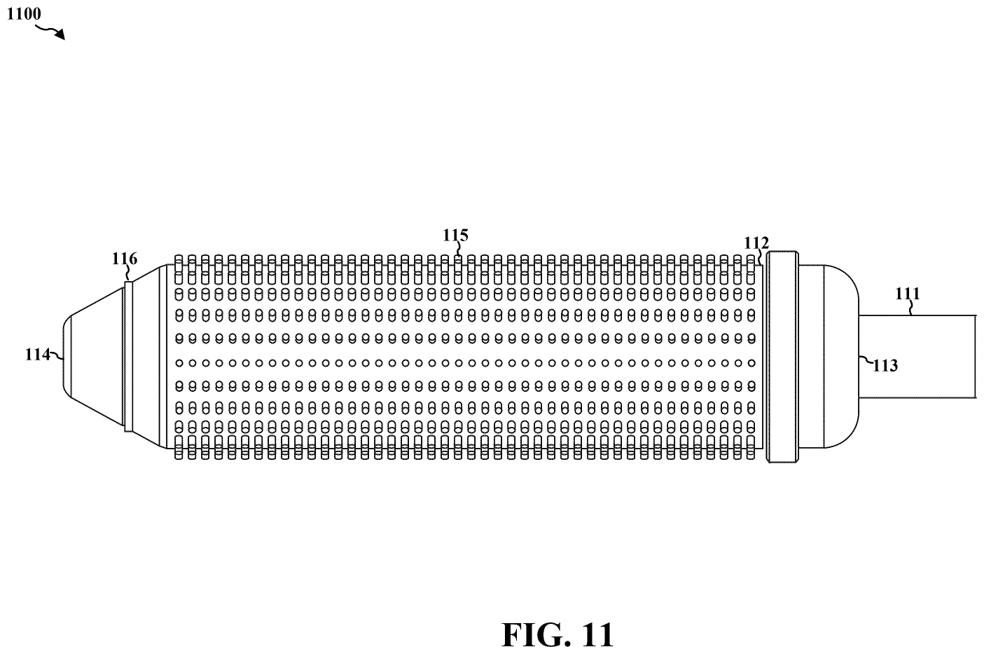

FIG. 11 is a diagram illustrating an example of a clot retrieval device 1100 having a plurality of bristles 115 that may each be a straight bristle that is aligned in a radial direction extending outwardly from a center of the elongated member 112. As shown in FIG. 11, a clot retrieval device 1100 may include a pusher 111 (partially shown) and an elongated member 112 having a proximal end 113 (e.g., non-distal end) and a distal end 114. In some aspects, the elongated member 112 may be referred to as a clot-engaging device. In some aspects, the elongated member 112 may be a polymer-based elongated member which may be made using polymer without metal or include a polymer coating on an outer surface of the elongated member 112. The elongated member 112 may have a plurality of bristles coupled to it. In the example shown in FIG. 11, the plurality of bristles may be straight bristles pointing outwardly from a center of the elongated member 112. Also, the elongated member 112 may include a radio-opaque marker 116. In some aspects, the clot retrieval device 1100 may fit a different type of blood vessel compared to the clot retrieval device 100. For example, clot retrieval device 1100 may have a size that would be fit for pulmonary vessels and the clot retrieval device 100 may have a size that would be fit for brain vessels. The example in FIG. 11 may be for exemplary purposes. More or less bristles may be used and bristles of various directions or shapes may be utilized for the clot retrieval device.

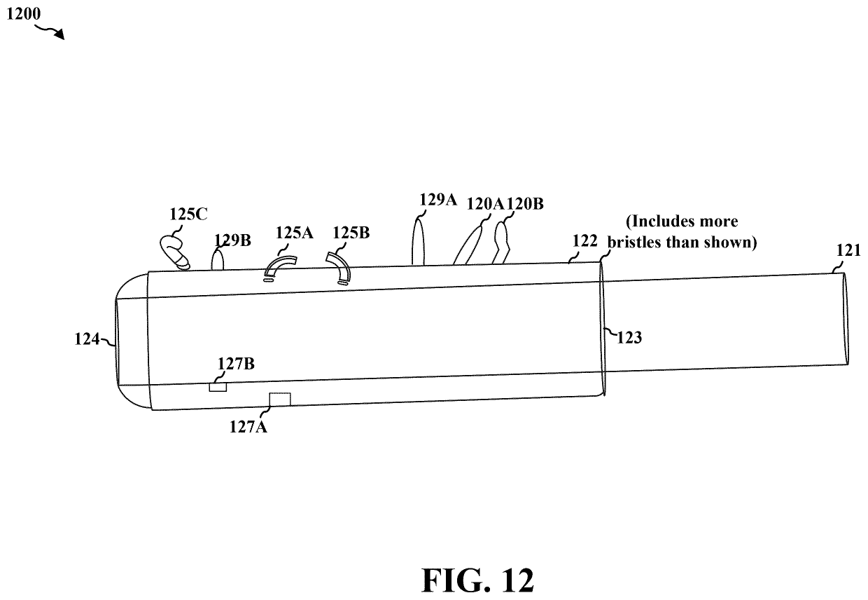

FIG. 12 is a diagram illustrating an example of a clot retrieval device 1200 with various example types of bristles. As shown in FIG. 12, a clot retrieval device may include a pusher 121 (partially shown) and an elongated member 122 having a proximal end 123 (e.g., non-distal end) and a distal end 124. In some aspects, the elongated member 122 may be referred to as a clot-engaging device. In some aspects, the elongated member 122 may be a polymer-based elongated member which may be made using polymer without metal or include a polymer coating on an outer surface of the elongated member 122. The elongated member 122 may have a plurality of bristles coupled to it. The bristles coupled to the elongated member 122 are shown for illustrative purposes and not all of the bristles coupled to the elongated member 122 are shown in FIG. 12. As illustrated in FIG. 12, there may be bristles of different types of shapes and lengths coupled to the elongated member 122, such as a straight bristle with a tapered end of a first length 129A, a straight bristle with a tapered end of a second length 129B, a first hook-shaped bristle 125A, a second hook-shaped bristle 125B, a third hook-shaped bristle 125C, a bent bristle 120A, and a zigzag bristle 120B. In addition, the elongated member 122 may also have a cavity 127A distributed along an outer surface of the elongated member 122. In some aspects, various cavities described herein, such as the cavity 127A, may be a cavity of any shape such as rectangular, cylindrical, square, elliptical, conical, spherical, oval, or the like. In some aspects where the elongated member 122 is hollow, a cavity 127B may be present on an inner surface of the elongated member 122. A number of different bristle shapes can be used in the clot retrieval device shown in FIG. 12. The example in FIG. 12 may be for exemplary purposes. More or less bristles may be used and bristles of various directions or shapes may be utilized for the clot retrieval device. In addition, there may be additional cavities, protrusions, or other structures distributed along an inner surface or an outer surface of the elongated member 122. In some aspects, a cavity may be able to hold a minute clot that might not be picked up (i.e., captured) by other components. In some aspects, a cavity may be able to hold a clot that breaks or falls apart.

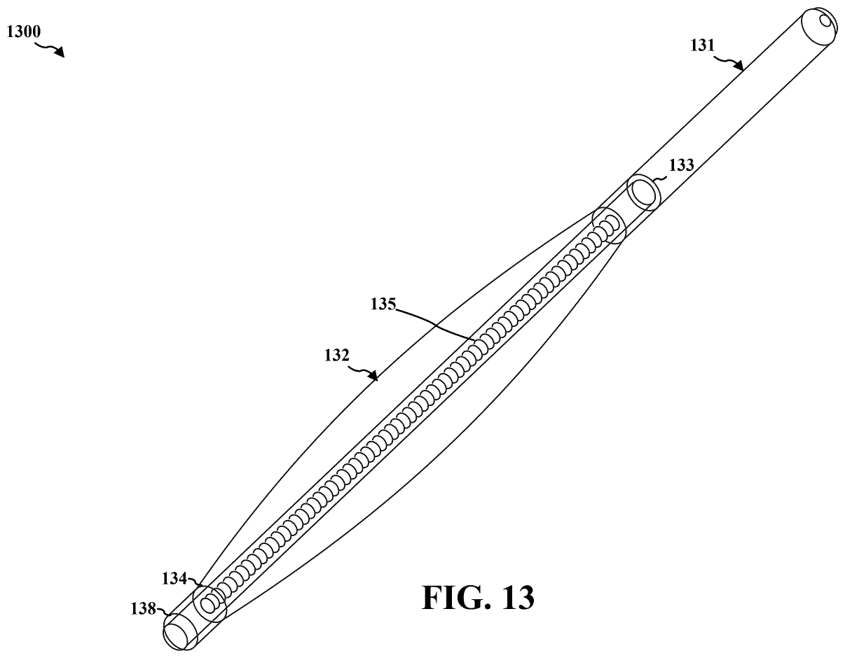

FIG. 13 is a diagram illustrating an example of the clot retrieval device 1300 that includes an inflatable balloon 132, in accordance with an example embodiment of the present disclosure. As illustrated in FIG. 13, the clot retrieval device 1300 may be coupled to a pusher 131. The second end 133 may be a proximal end and the first end 134 may be a distal end. The clot retrieval device 1300 may include a radio-opaque marker 138 for visualization so that an operator may determine whether the clot retrieval device 1300 has arrived at or is past the target. The example in FIG. 13 may be for exemplary purposes. The inflatable balloon may be configured differently and may be further attached to other elements, such as bristles, coils, or other material may be utilized for the clot retrieval device.

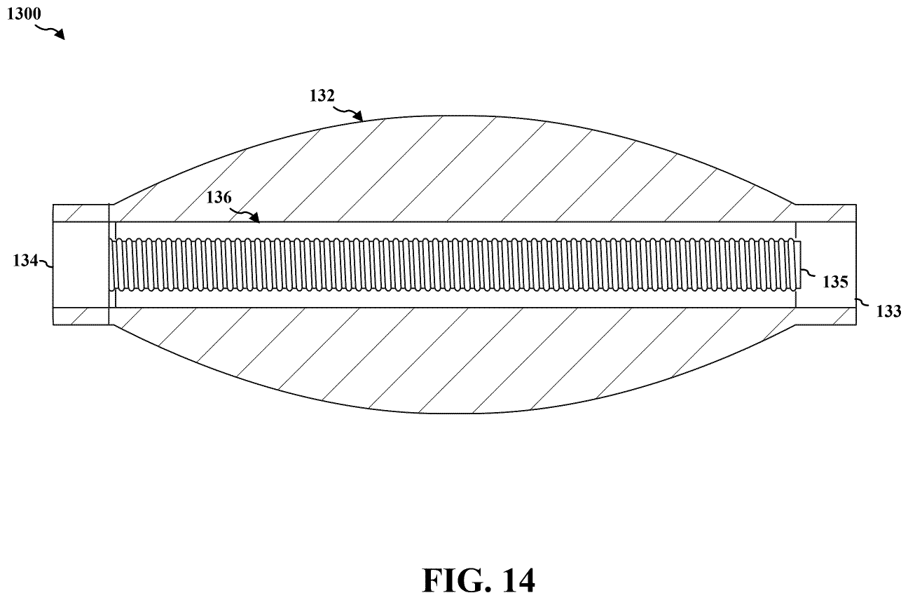

FIG. 14 is a diagram illustrating (cross-sectional) an example of a clot retrieval device 1300 that includes an inflatable balloon 132, in accordance with an example embodiment of the present disclosure. As illustrated in FIG. 14, the clot retrieval device 1300 may include the inflatable balloon 132. An inner surface within the inflatable balloon 132 may be coupled to an elongated member 136 that includes a spring shaft 135. The elongated member 136 may have a first end 134 and a second end 133. The first end 134 may be in a distal region of the clot retrieval device 1300 and the second end 133 may be in a proximal region of the clot retrieval device 1300. There may be internal lumen between the distal region and the proximal region. In other words, the clot retrieval device 1300 may include a proximal region, a distal region, and an internal lumen therebetween. In some aspects, the spring shaft 135 may be a spring coil shaft that may be used to load the inflatable balloon 132 into the elongated member. The inflatable balloon 132 may be inflated or deflated. When the clot retrieval device 1300 is moving towards a target material, the inflatable balloon 132 may be deflated. A state where an inflatable balloon is deflated may be referred to as a “deflated configuration” or “initial configuration” and a state where an inflatable balloon is inflated may be referred to as an “inflated configuration.” When the clot retrieval device 1300 arrives at the target material or past the target material, the inflatable balloon 132 may be inflated so that the clot retrieval device 1300 may engage with the target material. The inflated configuration may also be referred to as a “material-engagement configuration.” In such a configuration, the spring shaft 135 may be in a flexed configuration. After the clot retrieval device 1300 engages with the target material the inflatable balloon 132 may stay in the inflated configuration and the clot retrieval device 1300 may be pulled out so that the target material may also be pulled out. The example in FIG. 14 may be for exemplary purposes. The inflatable balloon may be configured differently and other elements may be distributed along an outer surface of the inflatable balloon, such as bristles, coils, protrusions, cavities, or other shapes that may be utilized for the clot retrieval device.

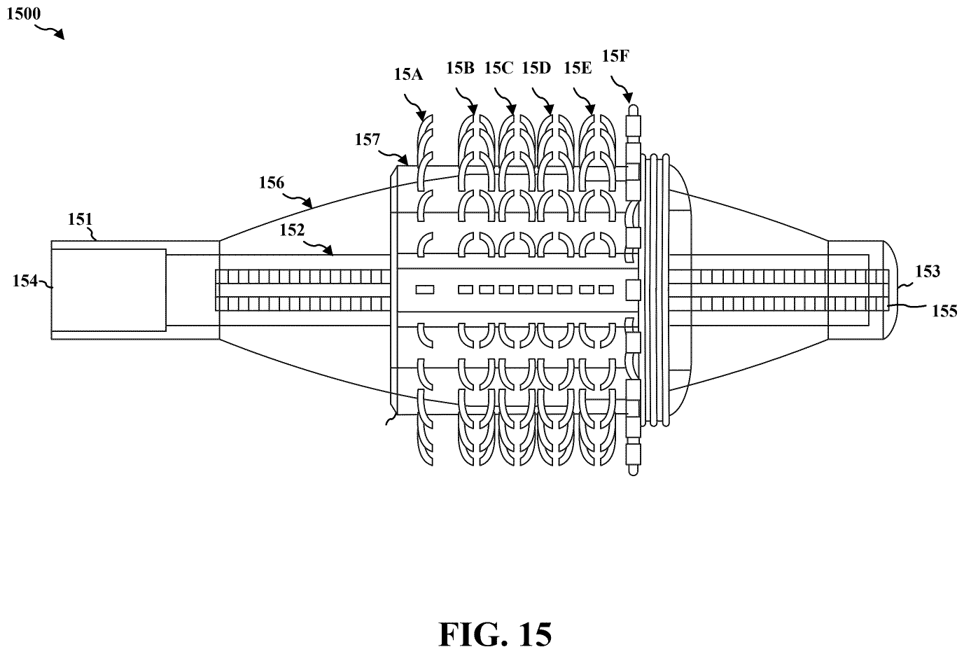

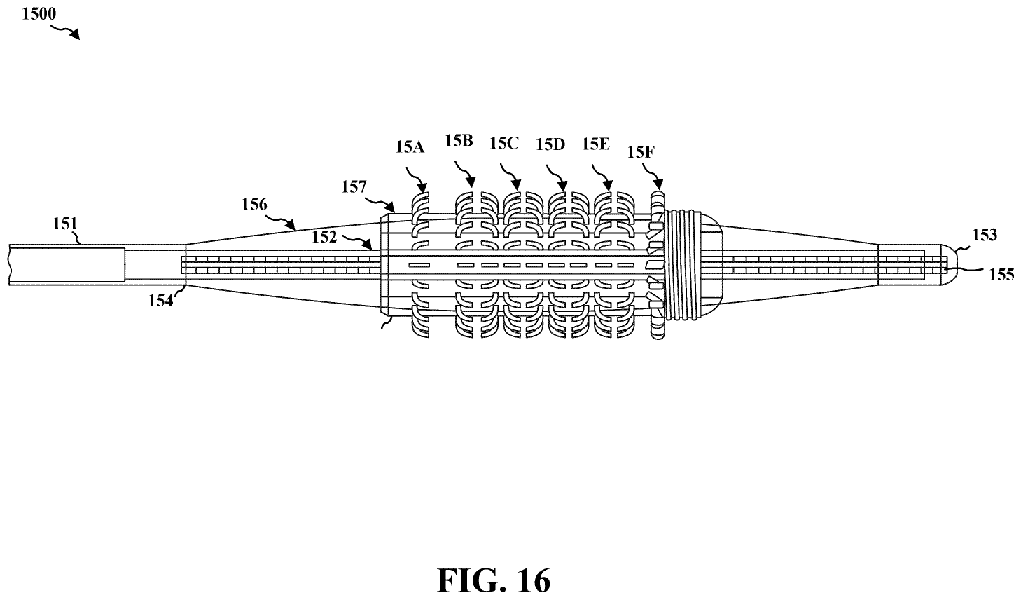

FIG. 15 is a diagram illustrating an example of a clot retrieval device 1500 in an inflated configuration that includes an inflatable balloon 156 and a plurality of bristles 15A-15F distributed along an outer surface of the inflatable balloon, in accordance with an example embodiment of the present disclosure. In some aspects, a plurality of bristles 15A-15F may be coupled to the inflatable balloon 156 by an intermediate layer 157, where the plurality of bristles is coupled to an exterior surface of the intermediate layer, and the inflatable balloon is coupled to an interior surface of the intermediate layer. In some aspects, the intermediate layer may include flexible material that remains coupled to the inflatable balloon when the inflatable balloon expands from a deflated configuration (e.g., FIG. 16) to an inflated configuration (e.g., FIG. 15), where the radius is greater in the inflated configuration than in the deflated configuration. As illustrated in FIG. 15, an inner surface within the inflatable balloon 156 may be coupled to an elongated member 152 that includes a spring shaft 155. The elongated member 152 may be coupled to a pusher 151. The elongated member 152 may have a first end 154 and a second end 153. In some aspects, the elongated member 152 may be referred to as a clot-engaging device. In some aspects, the spring shaft 155 may be a spring coil shaft that may be used to load the elongated member 152 into a catheter. The inflatable balloon 156 may be inflated or deflated. When the clot retrieval device 1500 is moving towards a target material, the inflatable balloon 156 may be deflated as shown in FIG. 15.

A plurality of bristles 15A-15F of various shape including a first set of bristles including hook-shaped bristle 15A pointing to the second end 153, a second set of bristles including a pair of hook-shaped bristles 15B, a third set of bristles including a pair of hook-shaped bristles 15C, a fourth set of bristles including a pair of hook-shaped bristles 15D, a fifth set of bristles including a pair of hook-shaped bristles 15D, and a sixth set of bristles including a non-hooked bristle 15F, may be coupled to an outer surface of an intermediate layer 157. The intermediate layer 157 may have its inner surface coupled to the inflatable balloon 156. As shown, the plurality of bristles is coupled to an exterior surface of the intermediate layer 157, and an outer surface of the inflatable balloon 156 is coupled to an interior surface of the intermediate layer 157. The example in FIG. 15 may be for exemplary purposes. The inflatable balloon may be configured differently and different elements, such as different bristles, different protrusions, or different cavities, or different intermediate layer may be utilized for the clot retrieval device. In some aspects, near the first end 154, there may be an inflation port associated with an inflation channel to inflate the inflatable balloon 156. In other words, the inflatable balloon 156 may be in fluid communication with (e.g., fluid may flow in or out of) the inflation port so that the inflatable balloon 156 may inflate or deflate.

FIG. 16 is a diagram illustrating an example of the clot retrieval device 1500 in a deflated configuration that includes an inflatable balloon and a plurality of bristles 15A-15F distributed along an outer surface of the inflatable balloon, in accordance with an example embodiment of the present disclosure. In some aspects, the aspect shown in FIG. 16 may be the clot retrieval device 1500 where the inflatable balloon 156 is in an deflated state. When the clot retrieval device 1500 arrives at the target material or past the target material, the inflatable balloon 156 may be inflated so that at least one bristle of the plurality of bristles on the clot retrieval device 1500 may engage with the target material. In some aspects, the inflatable balloon is configured to change the radius from an axial center of the elongated member 152 and the radius from the of the plurality of bristles from the axial center of the elongated member 152 may be accordingly changed (e.g., greater when inflated). The intermediate layer 157 may include flexible material that remains coupled to the inflatable balloon 156 when the inflatable balloon 156 expands from a deflated configuration to an inflated configuration. The example in FIG. 15 and FIG. 16 may be for exemplary purposes. The inflatable balloon may be configured differently and different elements, such as different bristles, different protrusions, or different cavities, or different intermediate layer may be utilized for the clot retrieval device.

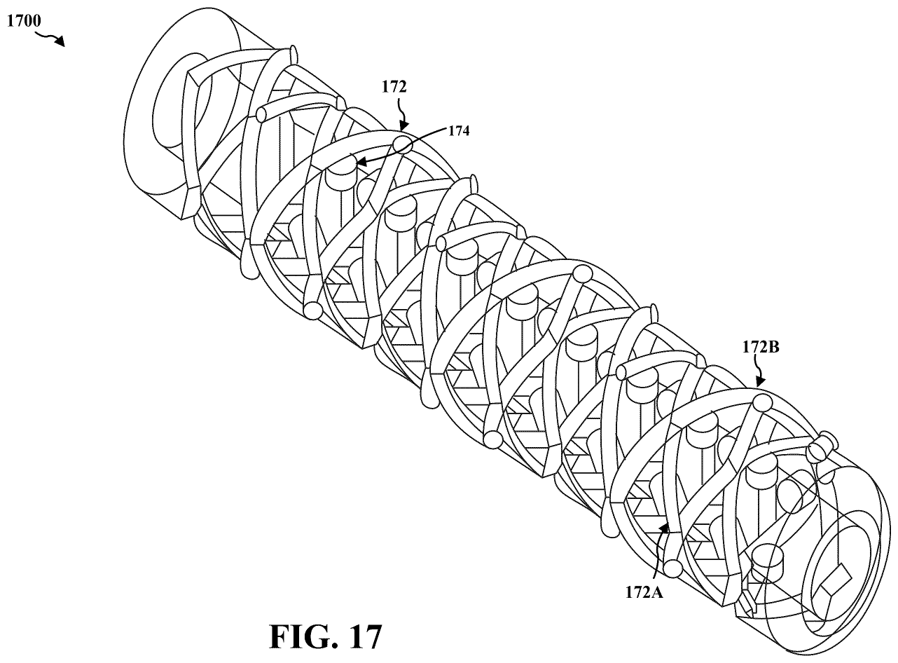

FIG. 17 is a diagram illustrating an example of a clot-engaging device 1700 with at least one helical semi-continuous protrusion or groove 172 and at least one cavity 174, in accordance with an example embodiment of the present disclosure. In some aspects, the clot-engaging device 1700 may be coupled to a pusher (not shown), such as any pusher described herein. As illustrated in FIG. 17, the at least one helical semi-continuous protrusion or groove 172 may include a first helical semi-continuous protrusion or groove 172A based on a first pattern (e.g., clockwise) and a second helical semi-continuous protrusion or groove 172B based on a second pattern (e.g., counterclockwise). The protrusion or groove may be considered to be semi-continuous because there may be a gap between each segment of the semi-continuous protrusion or groove. In some aspects, the at least one helical semi-continuous protrusion or groove 172 may be a groove. In some aspects, the clot-engaging device 1700 may be hollow and may include an inner lumen inside a tube body of the clot-engaging device 1700. As further shown in FIG. 17, the clot-engaging device 1700 may be hollow and the at least one cavity 174 may include cavities distributed along an outer surface of the clot-engaging device 1700 and/or cavities distributed along an inner surface of the clot-engaging device 1700.

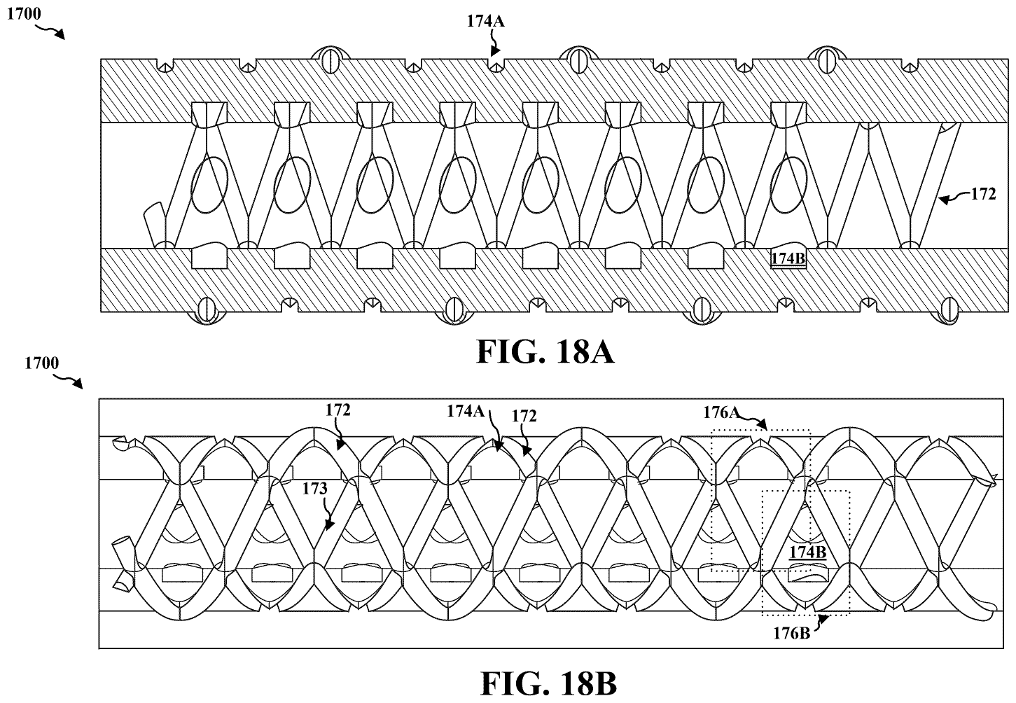

FIGS. 18A-18B are diagrams illustrating two different sideviews, cross-sectional and non-cross-sectional, of the example clot-engaging device 1700 with at least one helical semi-continuous protrusion or groove 172 and at least one cavity 174, in accordance with an example embodiment of the present disclosure. As illustrated in FIGS. 18A-18B, there may be one or more cavities including an example cavity 174A distributed along an outer surface of the clot-engaging device 1700. In some aspects, the one or more cavities including an example cavity 174A may be overlapping or non-overlapping with the at least one helical semi-continuous protrusion or groove (e.g., 172). In some embodiments, within the one or more cavities distributed along the outer surface of the clot-engaging device 1700, one or more additional shapes such as bristles, may be present. There may also be one or more cavities including an example cavity 174B distributed along an inner surface of the clot-engaging device 1700. As shown in FIGS. 18A-18B, there may be at least four (e.g., cight at bottom/top side) cavities spaced equally along the inner surface. In some aspects, the at least one helical semi-continuous protrusion or groove may form a plurality of kite-shaped regions, such as a kite-shaped region 176A and a kite-shaped region 176B, on the outer surface. For example, intersections of the at least one helical semi-continuous protrusion or groove may form a plurality of kite-shaped regions. As shown in FIG. 18B, a cavity, such as the cavity 174B, may be substantially centered within a kite-shaped region, such as the kite-shaped region 176B. In some aspects, the kite-shaped region may be shaped like a quadrilateral, a parallelogram, a kite, a trapezoid, a rhombus, a diamond, a rectangle, or a square. In some aspects, the at least one helical semi-continuous protrusion or groove 172 may be a groove and may have a first depth, that may be different (i.e., unequal to) or the same as a second depth of a second helical semi-continuous protrusion or groove 173.

In some aspects, the plurality of cavities may include a first cavity having a first volume, a second cavity having a second volume, and where the first volume and second volume are different or the same. For example, in some aspects, the cavity 174A may have a first volume that may be the same or different from (i.e., unequal to) a second volume of the cavity 174B. In some aspects, the first depth or the second depth may be a variable depth that changes (e.g., having a different depth on different parts of the semi-continuous groove). In some aspects, the first depth and/or the second depth may be less than a thickness of the clot-engaging device 1700. In some aspects, the clot-engaging device 1700 or a pusher connected to the clot-engaging device 1700 may include a radio-opaque marker. In some aspects, the plurality of cavities may include a first cavity having a first diameter, a second cavity having a second diameter, and where the first diameter and second diameter are different or the same. For example, in some aspects, the cavity 174A may have a first diameter that may be the same or different from (e.g., unequal to) a second diameter of the cavity 174B. By having cavities of different sizes, the clot-engaging device 1700 may be able to capture different foreign bodies (e.g., blood clot) of different sizes in the cavities of different sizes.

The example in FIG. 17 and FIGS. 18A-18B may be for exemplary purposes. Aspects where the elongated member may be non-hollow and the at least one helical semi-continuous protrusion or groove and at least one cavity may be distributed along an outer surface of the clot retrieval device may be possible. The inflatable balloon may be configured differently and different elements, such as different bristles, different protrusions, or different cavities, or different intermediate layers may be utilized for the clot retrieval device. In some aspects, the at least one helical semi-continuous protrusion or groove and at least one cavity may overlap with each other. In some aspects, the at least one helical semi-continuous protrusion or groove and at least one cavity may not overlap with each other. In some aspects, the at least one helical semi-continuous protrusion or groove and at least one cavity may be constructed in various materials, which may be the same or different. In some aspects, the at least one helical semi-continuous protrusion or groove and at least one cavity may be constructed based on sweep and cut. In some aspects, the at least one helical semi-continuous protrusion or groove and at least one cavity may be constructed based on molding. In some aspects, different segments of a semi-continuous protrusion or groove (e.g., 172 or 173) may be viewed as different protrusions or grooves and may be spaced substantially equidistant with one another on the outer surface.

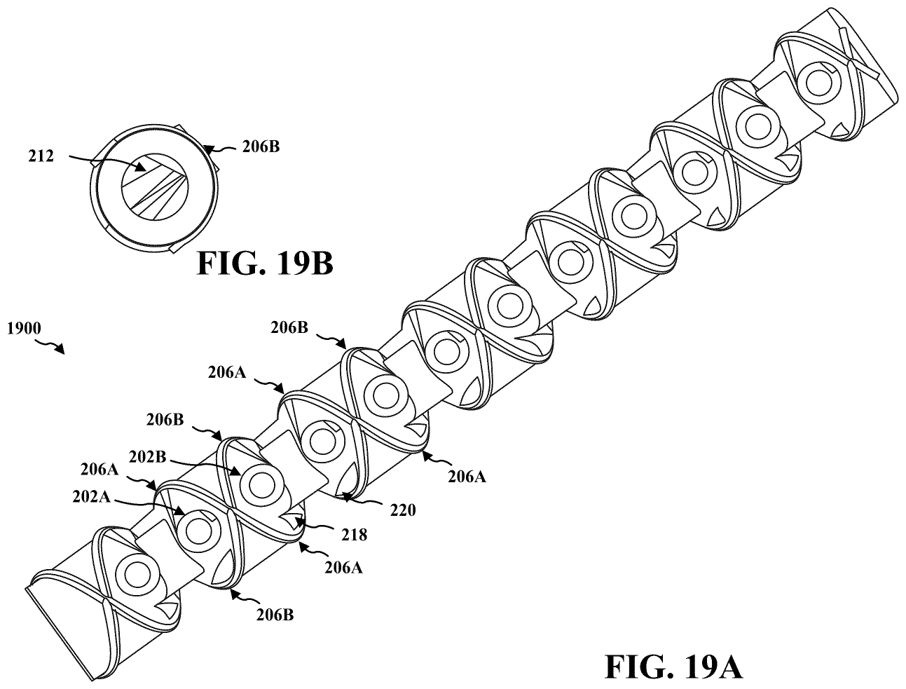

FIG. 19A is a diagram illustrating a perspective view of the example clot-engaging device 1900 with at least one helical semi-continuous protrusion or groove 206A/206B and at least one cavity 202A/202B, in accordance with an example embodiment of the present disclosure. As shown in FIG. 19A, there may be a first cavity 202A and a second cavity 202B on the top side of the outer surface. There may be two different helical protrusions distributed along the outer surface in different respective directions (e.g., counterclockwise or clockwise), helical semi-continuous protrusion or groove 206A and helical semi-continuous protrusion or groove 206B. There may also be one or more additional cavities including cavity 218 and cavity 220 on the outer surface. As shown in FIG. 19A, there may be at least four (e.g., twelve at top side) cavities spaced equally along the outer surface. Additionally, depicted in FIG. 19B is a cross-sectional view of a cross section of the clot-engaging device 1900. As shown in FIG. 19B, there may be one or more structures, such as structure 212 which may be coupled to the helical semi-continuous protrusion or groove 206A or the helical semi-continuous protrusion or groove 206B.

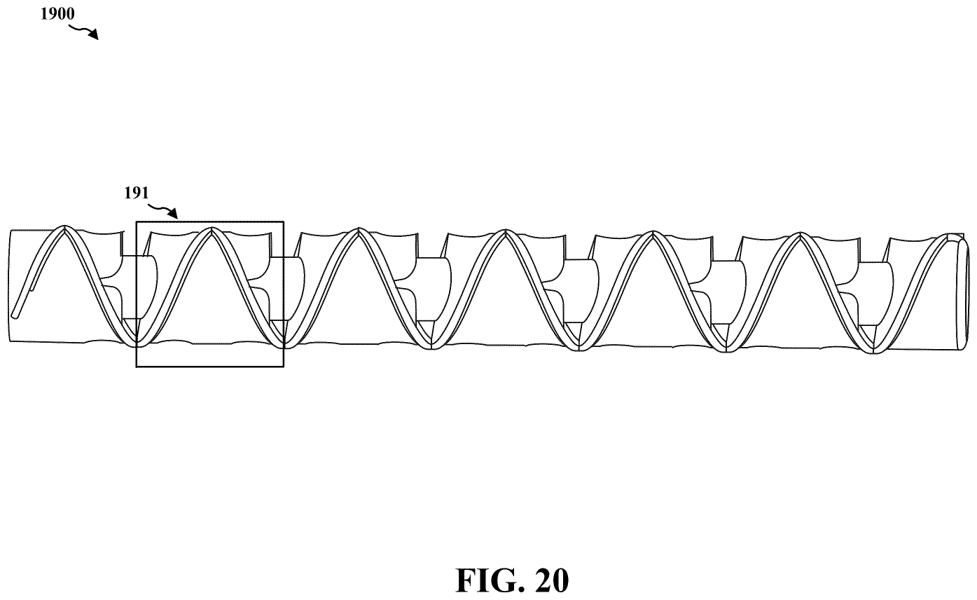

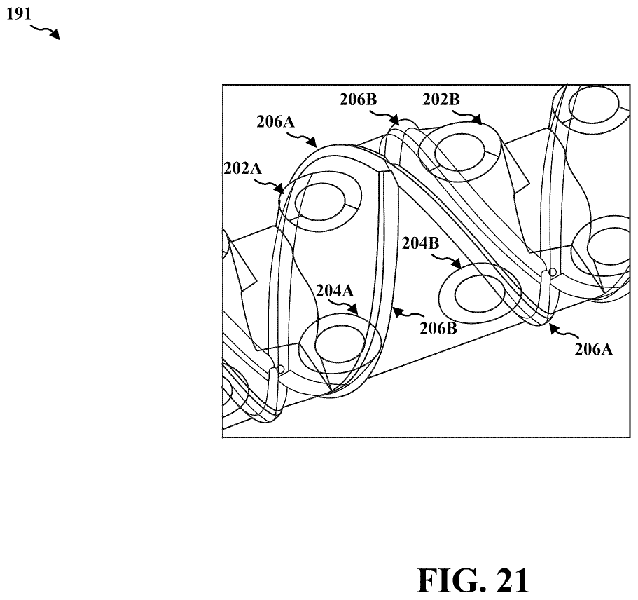

FIG. 20 is a diagram illustrating an example of a clot-engaging device 1900 with at least one helical semi-continuous protrusion or groove and at least one cavity, in accordance with an example embodiment of the present disclosure. As shown in FIG. 20, there may be one or more cavities and two different helical protrusions within a particular segment 191, which is shown in more detail in FIG. 21. In some aspects, the clot-engaging device 1900 may be hollow and may include an inner lumen.

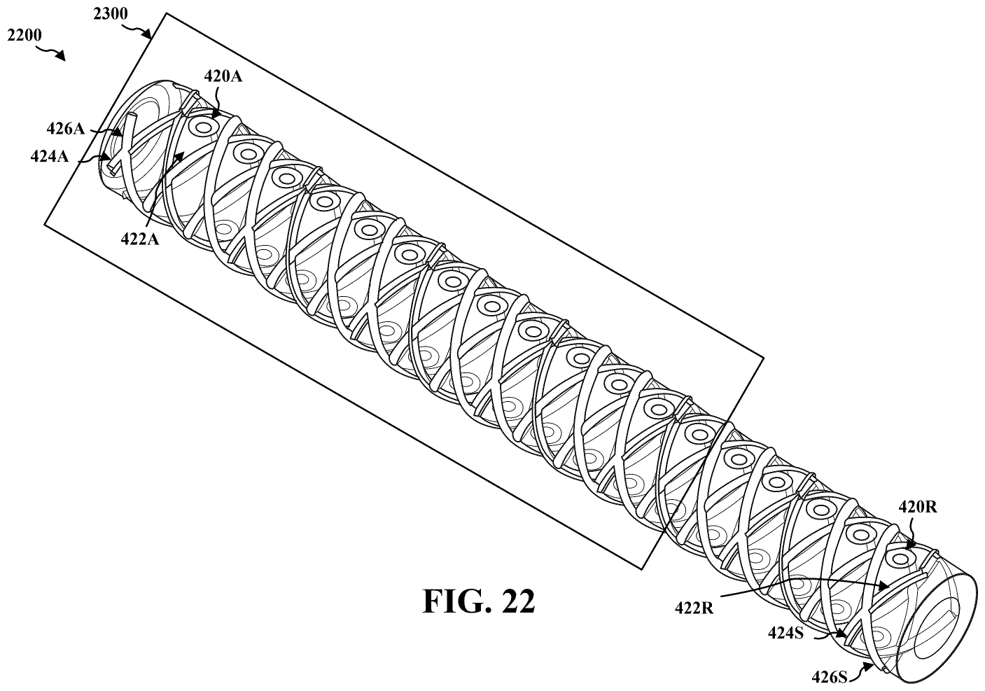

FIG. 21 is a diagram illustrating (cross-sectional) the particular segment 191 of the example clot-engaging device 1900 with at least one helical semi-continuous protrusion or groove and at least one cavity, in accordance with an example embodiment of the present disclosure. As shown in FIG. 21, within the particular segment 191, there may be a first cavity 202A and a second cavity 202B on the top side of the outer surface and a first cavity 204A and a second cavity 204B on the bottom side of the outer surface. There may be two different helical protrusions distributed along the outer surface in different respective directions (e.g., counterclockwise or clockwise), helical semi-continuous protrusion or groove 206A and helical semi-continuous protrusion or groove 206B. FIG. 22 is a diagram illustrating an example clot-engaging device 2200 with at least one helical semi-continuous protrusion or groove and at least one cavity, in accordance with an example embodiment of the present disclosure. As shown in FIG. 22, on the top side of the outer surface, there may be at least one cavity including cavities 420A to 420R. On the corresponding bottom side, there may be cavities corresponding to cavities 420A to 420R. There may be two different helical protrusions distributed along the outer surface in different respective directions (e.g., counterclockwise or clockwise), helical semi-continuous protrusion or groove 424A to 424S and helical semi-continuous protrusion or groove 426A and 426S. In some aspects, the at least one helical semi-continuous protrusion or groove may form a plurality of kite-shaped regions, such as the kite-shaped regions 422A to 422R, on the outer surface. For example, intersections of helical semi-continuous protrusion or groove 424A to 424S and helical semi-continuous protrusion or groove 426A and 426S may form a plurality of kite-shaped regions. As shown in FIG. 22, cavities, such as cavities 420A to 420R, may be substantially centered within the kite-shaped regions, such as the kite-shaped regions 422A to 422R. In some aspects, the kite-shaped regions may be shaped like a quadrilateral, a parallelogram, a kite, a trapezoid, a rhombus, a diamond, a rectangle, or a square.

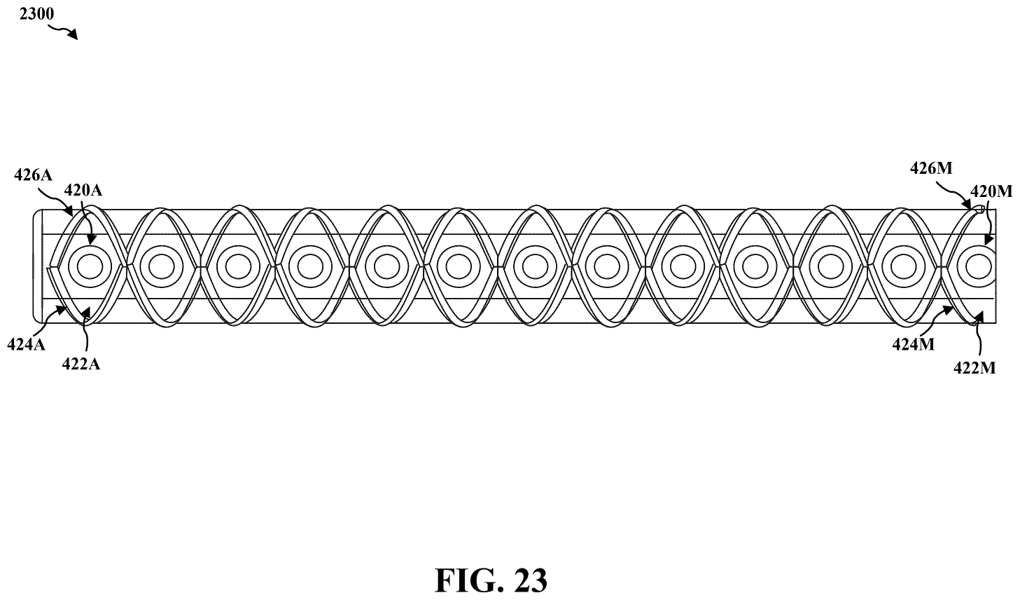

FIG. 23 is a diagram illustrating a side view of a section 2300 of the example clot-engaging device 2200 with at least one helical semi-continuous protrusion or groove and at least one cavity, in accordance with an example embodiment of the present disclosure. As shown in FIG. 23, within the section 2300, there may be at least one cavity including cavities 420A to 420M. On the corresponding bottom side, there may be cavities corresponding to cavities 420A to 420M. There may be two different helical protrusions distributed along the outer surface in different respective directions (e.g., counterclockwise or clockwise), helical semi-continuous protrusion or groove 424A to 424M and helical semi-continuous protrusion or groove 426M and 426M. As shown in FIG. 23, cavities, such as cavities 420A to 420M, may be substantially centered within the kite-shaped regions, such as the kite-shaped regions 422A to 422M. In some aspects, the kite-shaped regions may be shaped like a quadrilateral, a parallelogram, a kite, a trapezoid, a rhombus, a diamond, a rectangle, or a square.

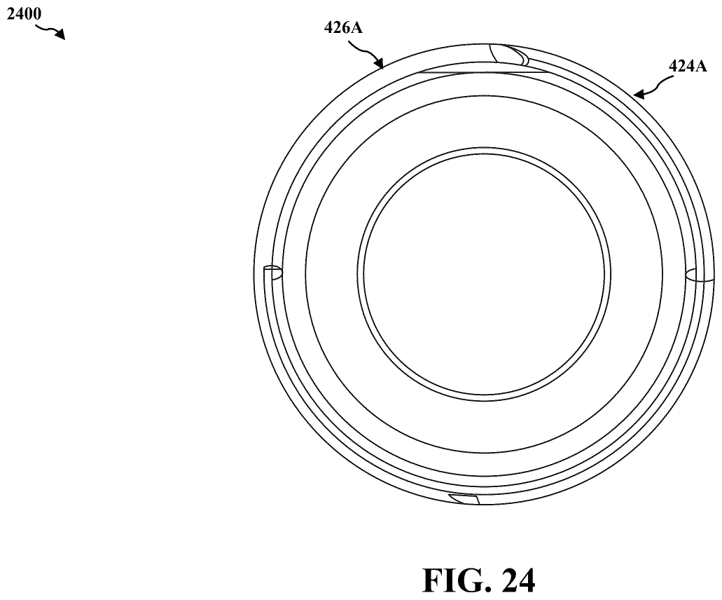

FIG. 24 is a diagram 2400 illustrating a top view of the example clot-engaging device 2200 with at least one helical semi-continuous protrusion or groove and at least one cavity, in accordance with an example embodiment of the present disclosure.

The example in FIGS. 19A-19B, FIG. 20, FIG. 21, FIG. 22, FIG. 23, and FIG. 24 may be for exemplary purposes. Aspects where the elongated member may be non-hollow and the at least one helical semi-continuous protrusion or groove and at least one cavity may be distributed along an outer surface of the clot retrieval device may be possible. The inflatable balloon may be configured differently. Different elements, such as different bristles, different protrusions, or different cavities, or different intermediate layer may be utilized for the clot retrieval device. In some aspects, the at least one helical semi-continuous protrusion or groove and at least one cavity may overlap with each other. In some aspects, the at least one helical semi-continuous protrusion or groove and at least one cavity may not overlap with each other.

In some aspects, the at least one cavity may be an array of cavities spaced apart (e.g., 202A, 202B, and additional cavities) from one another. In some aspects, the at least one helical semi-continuous protrusion or groove and at least one cavity may be constructed in various materials, which may be the same or different. In some aspects, the at least one helical semi-continuous protrusion or groove or the at least one cavity may be constructed based on sweep and cut. In some aspects, the at least one helical semi-continuous protrusion or groove or the at least one cavity may be constructed based on molding. In some aspects, the clot-engaging device 1900 or a pusher connected to the clot-engaging device 1900 may include a radio-opaque marker.

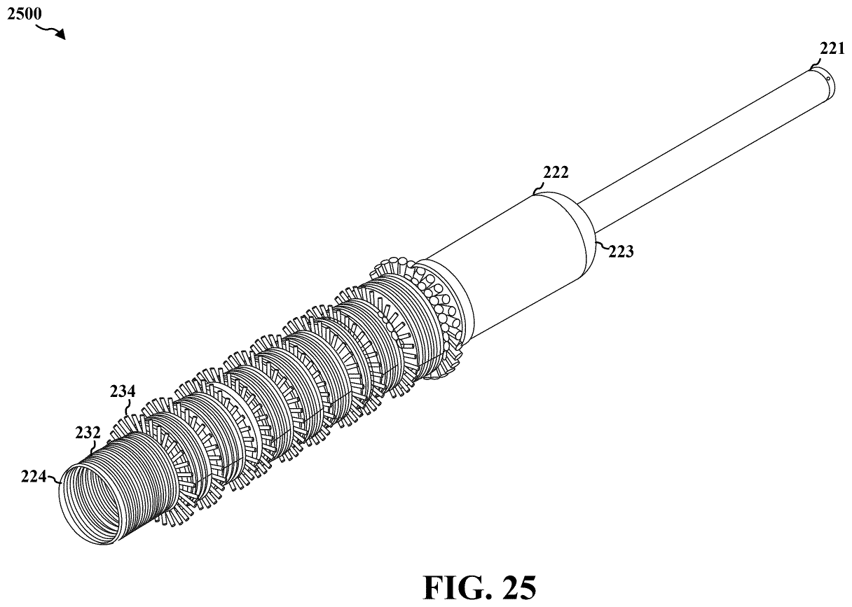

FIG. 25 is a diagram illustrating an example of a clot retrieval device 2500 having a plurality of bristles 234 and a plurality of coils 232, in accordance with an example embodiment of the present disclosure. As shown in FIG. 22, a clot retrieval device 2500 may include a pusher 221 (partially shown) and an elongated member 222 having a proximal end 223 (e.g., non-distal end) and a distal end 224. In some aspects, the elongated member 222 may be a polymer-based elongated member which may be made using polymer without metal (or other non-metal materials), made using polymer and metal, or include a polymer coating on an outer surface of the elongated member 222. The elongated member 222 may have a plurality of bristles and a plurality of coils distributed along the outer surface.

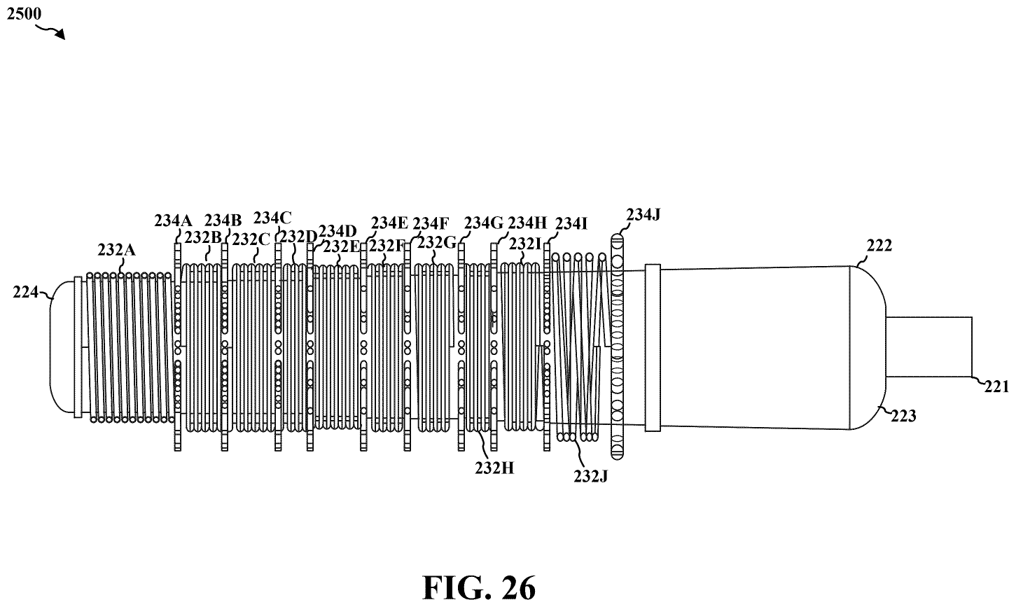

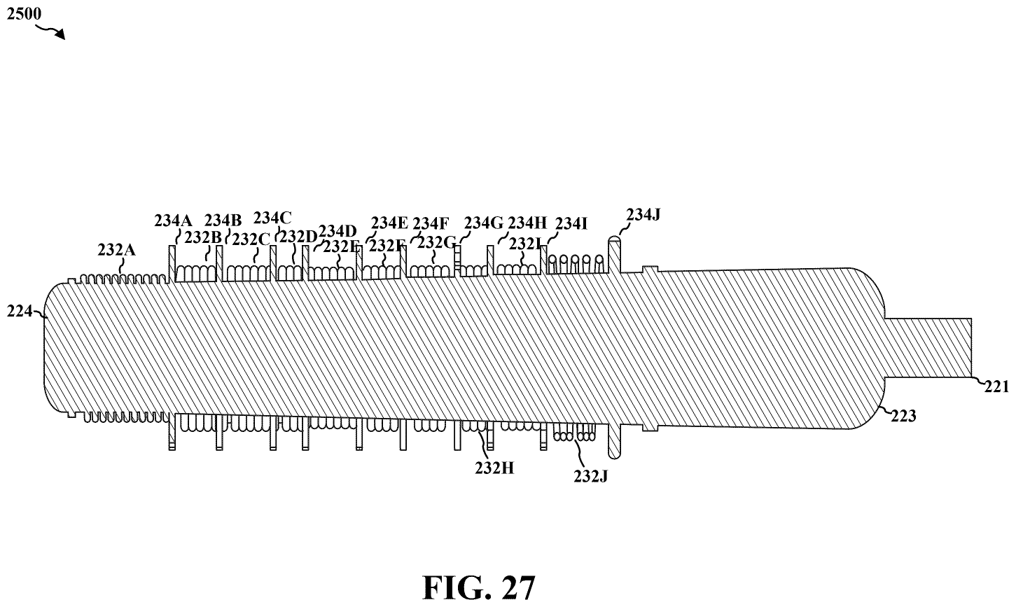

FIG. 26 is a diagram illustrating a sideview of an example of the clot retrieval device 2500 having a tapered distal end 224, a plurality of bristles 234A-234J, and a plurality of coils 232A-232J, in accordance with an example embodiment of the present disclosure. FIG. 27 is a diagram illustrating a cross-sectional cut view of the example the clot retrieval device 2500 in FIG. 26 having a plurality of bristles and a plurality of coils, in accordance with an example embodiment of the present disclosure. As illustrated in FIG. 26 and FIG. 27, the plurality of coils distributed along the outer surface of the elongated member 222 may include a first set of coils 232A, a second set of coils 232B, a third set of coils 232C, a fourth set of coils 232D, a fifth set of coils 232E, a sixth set of coils 232F, a seventh set of coils 232G, an eighth set of coils 232H, a ninth set of coils 232I, and a tenth set of coils 232J. As illustrated in FIG. 26 and FIG. 27, the plurality of bristles distributed along the outer surface of the elongated member 222 may include a first set of bristles 234A, a second set of bristles 234B, a third set of bristles 234C, a fourth set of bristles 234D, a fifth set of bristles 234E, a sixth set of bristles 234F, a seventh set of bristles 234G, an eighth set of bristles 234H, a ninth set of bristles 234I, and a tenth set of bristles 234J.

A region having bristles may be referred to as a “bristle region” and a region without bristles may be referred to as a “bristle-free region.” In some aspects, the bristle-free region may include coils and may be referred to as “a coil region.” Each set of bristles may be separated by a set of coils and vice versa. As a particular example, each of the at least one coil including the first set of coils 232A, the second set of coils 232B, the third set of coils 232C, the fourth set of coils 232D, the fifth set of coils 232E, the sixth set of coils 232F, the seventh set of coils 232G, the eighth set of coils 232H, the ninth set of coils 232I, and the tenth set of coils 232J, is located in one of a plurality of coil regions on the surface. In some aspects, each of the plurality of coil regions is wrapped around a circumference of the elongated member 222. In some aspects, each of the plurality of coil regions is proximal to at least one bristle of the plurality of bristles (e.g., a region including the first set of coils 232A is proximal to the first set of bristles 234A), and each of the plurality of coil regions is distal to at least one other bristle of the plurality of bristles (e.g., a region including the first set of coils 232A is distal to the tenth set of bristles 234J). In some aspects, the coils may be made using polymer without metal (or other non-metal materials), made using polymer and metal, or include a polymer coating on an outer surface of the coil. In some aspects, the bristles may be made using polymer without metal (or other non-metal materials), made using polymer and metal, or include a polymer coating on an outer surface of the coil.

The example shown in FIG. 25, FIG. 26, and FIG. 27 is merely shown as an example. There may be additional set(s) of coils or bristles placed next to other bristles or coils. Also, there may be back-to-back sets of coils or bristles. The coils or bristles used herein may be various types of coils or bristles as described herein. For example, a coil may be constructed from a polymer based material, a metal based material, or the like.

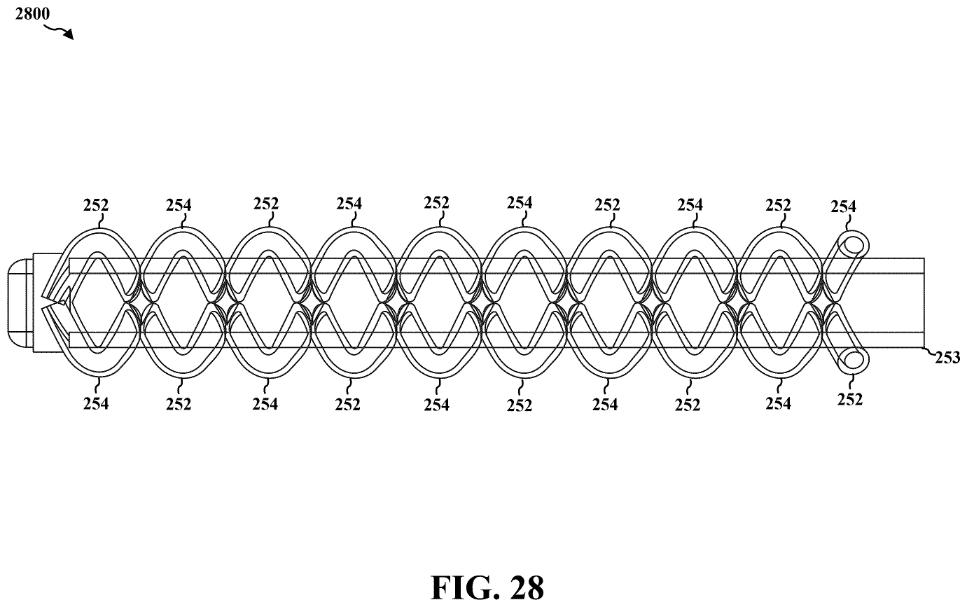

FIG. 28 is a diagram illustrating a sideview of an example of a clot retrieval device 2800 having a plurality of helical protrusions, in accordance with an example embodiment of the present disclosure. As shown in FIG. 28, on an outer surface of the elongated member 253 of the clot retrieval device 2800, there may be two different helical protrusions distributed along the outer surface in different respective directions (e.g., counterclockwise or clockwise directions), helical semi-continuous protrusion or groove 252 and helical semi-continuous protrusion or groove 254.

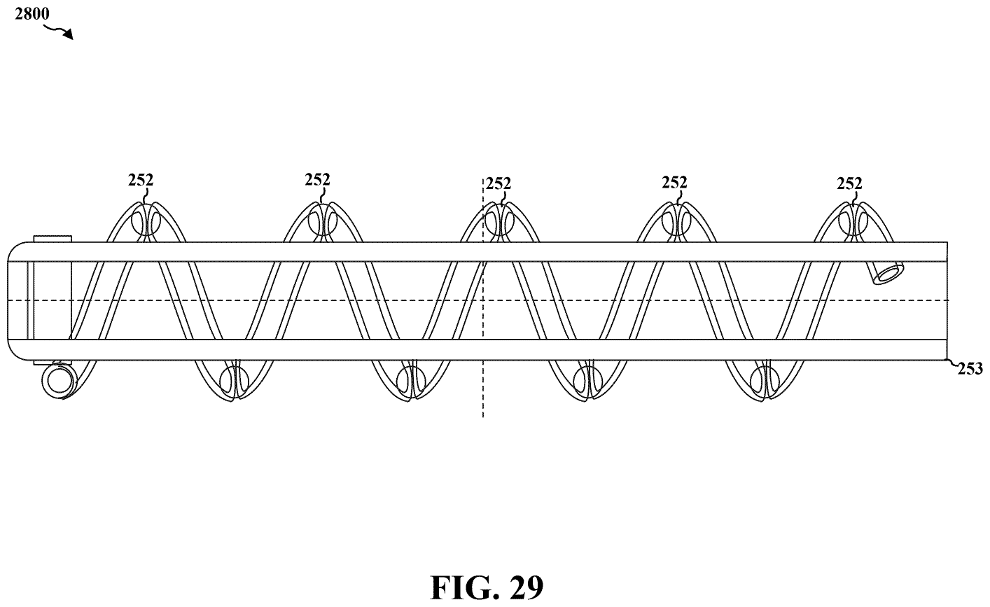

FIG. 29 is a diagram illustrating a sideview (cross-sectional) of an example of the clot retrieval device 2800 having a plurality of helical protrusions, in accordance with an example embodiment of the present disclosure. In FIG. 29, the helical semi-continuous protrusion or groove 252 is shown and there may be additional semi-continuous protrusions or grooves.

The example shown in FIG. 28 and FIG. 29 may be merely shown as an example. There may be coils or bristles, cavities, or helical protrusions that may overlap or not overlap with the helical semi-continuous protrusion or groove 252 and the helical semi-continuous protrusion or groove 254 being distributed along the outer surface of the elongated member 253. In some aspects, different segments of a semi-continuous protrusion or groove (e.g., 252, 253) may be viewed as different protrusions or grooves and may be substantially equidistant with one another. In some aspects, there may be additional pairs of semi-continuous protrusions or grooves. In some aspects, there may be at least two pairs of semi-continuous protrusions or grooves. In some aspects, there may be at least three pairs of semi-continuous protrusions or grooves.

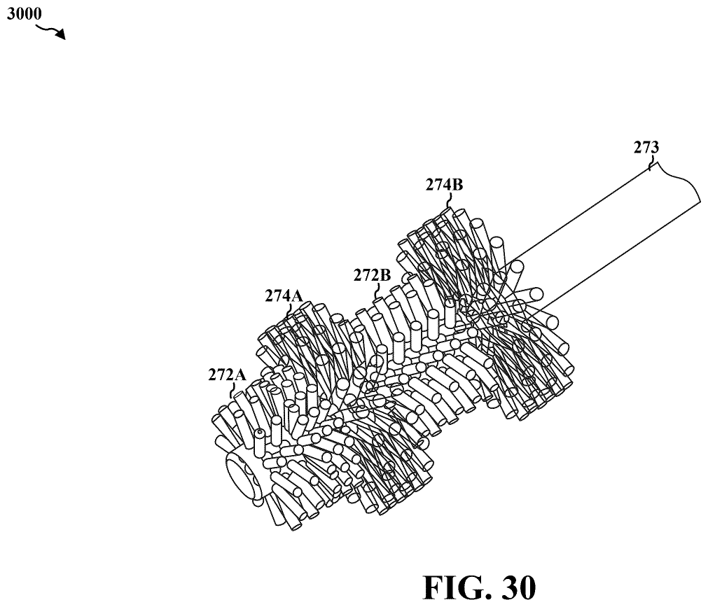

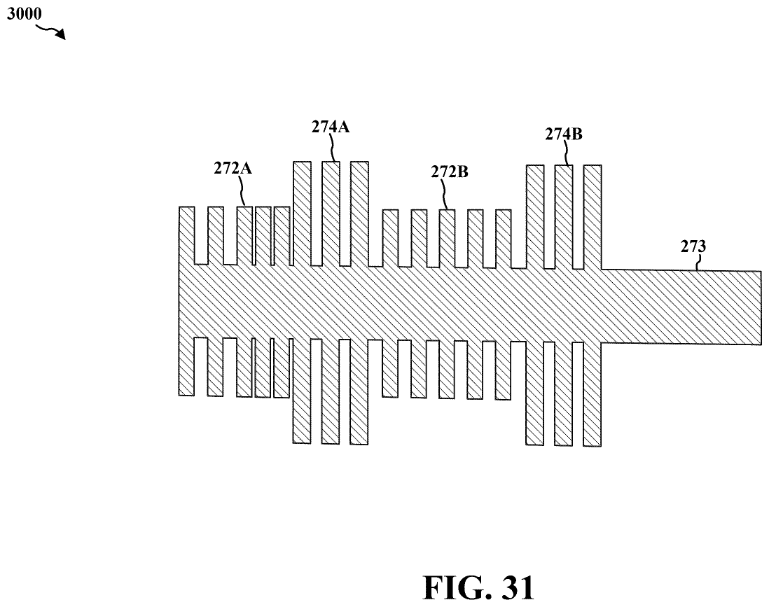

FIG. 30 is a diagram illustrating a perspective view of an example of a clot retrieval device 3000 having a plurality of bristles 272A/272B and 274A/274B of various lengths, in accordance with an example embodiment of the present disclosure. FIG. 31 is a diagram illustrating a cross-sectional cut view of the example clot retrieval device 3000 in FIG. 30 having a plurality of bristles of various lengths, in accordance with an example embodiment of the present disclosure. As shown in FIG. 30 and FIG. 31, on an elongated member 273 of the clot retrieval device 3000, there may be a first set of bristles 272A of a first length and a second set of bristles 272B of the first length, and there may be a first set of bristles 274A of a second length and a second set of bristles 274B of the second length. As an example, the first length may be smaller than the second length. In some aspects, the first length and the second length may be a first substantially uniform length within a first range and a second substantially uniform length within a second range. The first range may be different from the second range.

The example shown in FIG. 30 and FIG. 31 may be merely shown as an example. More or less bristles may be utilized, and more or less bristles along each respective sets of bristles may be utilized for the clot retrieval device. The bristles may be associated with different body shapes, different tip shapes, different widths, different directions or lengths.

In some aspects, when bristles are used, the tip shape of each bristle may be one of rounded, circular, square, rectangular, polygonal, triangular, helical, oval, or trapezoidal, and may be different or the same within a same set of bristles or across different sets of bristles. The body shape may be one of straight, zigzagged, or bent, and may be different or the same within a same set of bristles or across different sets of bristles. The material composition of bristles may be polymer, polymer-based, nickel titanium, or other suitable material based on a Shore hardness or durometer range. In some aspects, a curved distal bristle tip of a hook-shaped bristle may have a curvature of at least 90 degrees, 135 degrees, or 180 degrees. The bristles may be pointing towards different directions, such as pointing linearly in a radial direction from or toward a center of the clot-engaging device or pointing in a tangential direction away from the outer surface or an inner surface of the clot-engaging device.

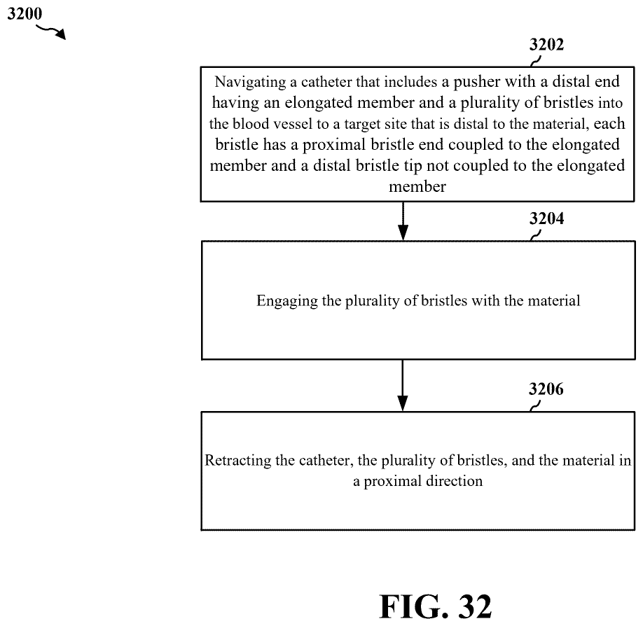

FIG. 32 is a flowchart 3200 of a method of removing a target material (e.g., foreign body such as a blood clot) from a blood vessel using a clot retrieval device (e.g., 100, 600, 700, 800, 1100, 1200, 1500, 1700, 1900, 2500, 2800, or 3000). It should be understood that this exemplary method is not meant to be limiting.

At 3202, the method may include navigating a catheter (e.g., microcatheter) into the blood vessel to a target site that is distal to the material. In some aspects, a chosen clot retrieval device such as any of the clot retrieval devices described or suggested herein is advanced through a lumen of the catheter and deployed near or at the target site. In some aspects, the clot retrieval device may be chosen based on one or more of: (1) a size of the blood vessel, (2) a morphology of the material, (3) a location of the material (e.g., against the arterial wall or at the center of the blood vessel), (4) a size of the material, or the like.

In some aspects, the clot retrieval device includes a pusher (e.g., 11, or the like) with a distal end having an elongated member (e.g., 12, 62, 72, 82, 112, 122, 152, 222, 273, or the like) and a plurality of bristles (e.g., 1A-1S, 1B1-1B8, 21-28, 6A-6J, 7A-7K, 8A-8W, 115, bristles illustrated in FIG. 12, 234, 272A-274B, or the like). In some aspects, each bristle has a proximal bristle end coupled to the elongated member and a distal bristle tip not coupled to the elongated member. In some aspects, the plurality of bristles includes a first set of bristles coupled to a first portion of the elongated member and second set of bristles coupled to a second portion of the elongated member. In some aspects, the first portion may be a first longitudinal portion. In some aspects, the first portion may be a first cross-sectional portion. In some aspects, the second portion may be a second longitudinal portion. In some aspects, the second portion may be a second cross-sectional portion. In some aspects, in a delivery configuration, the medical device may be within a lumen of a catheter, and in a deployed configuration, the medical device may be distal to the catheter. In some aspects, each bristle of the plurality of bristles may have a rounded tip (e.g., 1A-1S, 1B1-1B8, 21-28, 6A-6J, 7A-7K, 8A-8W, 115, bristles illustrated in FIG. 12, 234, 272A-274B, or the like) and the elongated member may be polymer-based. In some aspects, the first set of bristles may be parallel to the second set of bristles (e.g., as illustrated in FIGS. 1-12).

In some aspects, each bristle of the plurality of bristles may have a straight bristle portion that is aligned in a radial direction extending outwardly from a center of the elongated member (e.g., as illustrated in FIG. 9 and FIG. 10) or toward a center of the elongated member (e.g., similar to the ones illustrated in FIG. 5A and FIG. 5B but extending toward the center instead of tangentially extending). In some aspects, the straight bristle portion of each bristle of the plurality of bristles may be located at least in part at (e.g., or near) the proximal bristle end of the bristle.

In some aspects, at least one of the first set of bristles or the second set of bristles may include hook-shaped bristles (e.g., as illustrated in FIG. 8, FIG. 9, and FIG. 12), and each of the hook-shaped bristles may have a curved distal bristle tip, where the curved distal bristle tip may point to the distal end or a non-distal end (e.g., as illustrated in FIG. 6 and FIG. 7, such as 6A-6J or 7B-7H). In some aspects, at least one of the first set of bristles or the second set of bristles may include just the hook-shaped bristles (e.g., 6A-6J), and each of the hook-shaped bristles may have the curved distal bristle tip. In some aspects, the first set of bristles or the second set of bristles may include the hook-shaped bristles and bristles of different shapes (e.g., as illustrated in FIG. 9 and FIG. 12), such as a circular shape, a square shape, a rectangular shape, a polygon shape, a triangular shape, an oval shape, a trapezoid shape, a straight line (e.g., 129A) longitudinally, a zigzag line (e.g., 120B) longitudinally, and/or a bent line (e.g., 120A) longitudinally.

In some aspects, the curved distal bristle tip of each of the hook-shaped bristles may have a curvature of at least 90 degrees. In some aspects, the curved distal bristle tip of each of the hook-shaped bristles may have a curvature of at least 135 degrees. In some aspects, the curved distal bristle tip of each of the hook-shaped bristles may have a curvature of at least 180 degrees. In some aspects, the curved distal bristle tip of each of the hook-shaped bristles may have a helical shape. In some aspects, each bristle of the plurality of bristles may be hook-shaped bristles (e.g., as illustrated in FIG. 6 and FIG. 7, such as 6A-6J or 7B-7H), and each of the hook-shaped bristles may have a curved distal bristle tip, where the curved distal bristle tip may point to the distal end or a non-distal end of the elongated member (e.g., 11). In some aspects, the elongated member may be made of polymer. In some aspects, the elongated member may be polymer-based (e.g., includes polymer).

In some aspects, the first portion and the second portion may be located at a tapered region of the elongated member (e.g., as illustrated in FIG. 8, FIG. 9, and FIG. 10). In some aspects, the first portion and the second portion may be cross-sectional portions and the first cross-sectional portion and the second cross-sectional portion may be located at a tapered region of the elongated member. In some aspects, the first portion and the second portion may be longitudinal portions and the first longitudinal portion and the second longitudinal portion may be located at a tapered region of the elongated member (e.g., 82). In some aspects, the first cross-sectional portion of the elongated member (e.g., 82) may have a first diameter and the second cross-sectional portion of the elongated member may have a second diameter. In some aspects, the first diameter may be larger than the second diameter.

In some aspects, each bristle in the first set of bristles (e.g., 272A, 272B) may have a first length, where each bristle in the second set of bristles (e.g., 274A, 274B) may have a second length, and the first length may be equal to, greater than, or less than the second length (e.g., as illustrated in FIG. 8, FIG. 9, and FIG. 10).

In some aspects, a first subset of bristles (e.g., 272A) in the first set of bristles and a second subset of bristles (e.g., 272B) in the second set of bristles may have a first length, a third subset of bristles (e.g., 274A) in the first set of bristles and a fourth subset of bristles (e.g., 274B) in the second set of bristles may have a second length, and the first length may be less than or greater than the second length. In some aspects, at least one bristle of the plurality of bristles may be pointing in a tangential direction away from a circle associated with the elongated member (e.g., as illustrated in FIG. 1).

At 3204, the method may include engaging the plurality of bristles with the material. For example, when the clot retrieval device is near the target site and the microcatheter is withdrawn to deploy the clot retrieval device, the plurality of bristles may touch the material, so that the material may be retrieved from the blood vessel. In some aspects, the material is trapped with the bristles or cavity of clot retrieval device, thus enabling the material to be removed from the target site. In some aspects, the material may be trapped between hook-shaped bristles such as 7G and 7F or 6G and 6F. In some aspects, the material may be trapped in a cavity, such as any particular cavity including cavity 174A, 174B, 202A, 202B, 218, 220, or 420A to 420R. In some aspects, the material may be trapped within any particular protrusion or groove such as 172, 173, 206A, 206B, 212, 252, 254, 424A to 424S, or 426A to 426S. In some aspects, to engage the plurality of bristles with the material, the medical device may be in the deployed configuration where the clot retrieval device (e.g., medical device) is no longer constrained within the lumen of the catheter and may be distal to the catheter.

At 3206, the method may include retracting the catheter, the plurality of bristles, and the material in a proximal direction. For example, an operator may pull the catheter along the proximal direction to remove the material from a blood vessel. In some aspects, the blood vessel may be an artery (e.g., a brain artery or a pulmonary artery).

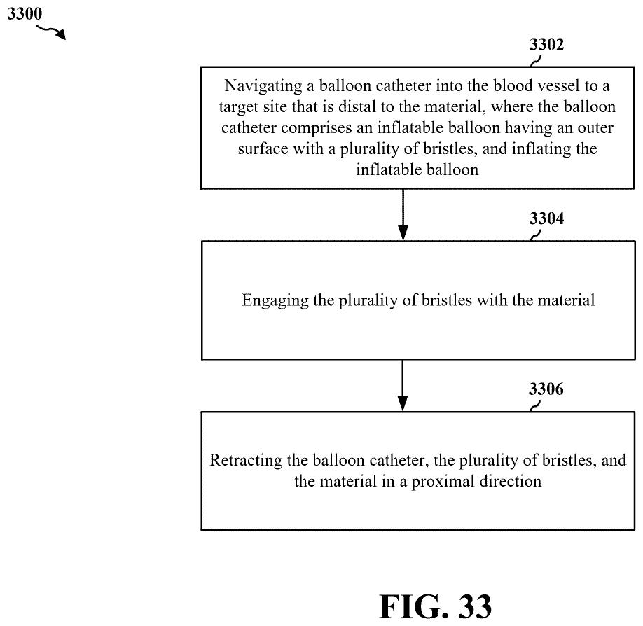

FIG. 33 is a flowchart 3300 of a method of removing a target material (e.g., foreign body such as a blood clot) from a blood vessel using a clot retrieval device that includes an inflatable balloon (e.g., 1500). It should be understood that this exemplary method is not meant to be limiting.

At 3302, the method may include navigating a balloon catheter into the blood vessel to a target site that is near or distal to the material, where the balloon catheter includes an inflatable balloon having an outer surface with a plurality of bristles (e.g., clot retrieval device 1500, FIG. 15-16). In some aspects, at 3302, when the balloon catheter is near or distal to the material, the balloon catheter is inflated (e.g., FIG. 15). In some aspects, the balloon catheter may include a chosen clot retrieval device from any of the clot retrieval devices described or suggested herein (e.g., with bristle, cavity, protrusion, or grooves). In some aspects, the clot retrieval device may be chosen based on one or more of: (1) a size of the blood vessel, (2) a morphology of the material, (3) a location of the material (e.g., against the arterial wall or at the center of the blood vessel), (4) a size of the material, or the like. In some aspects, each bristle of the plurality of bristles includes at hook-shaped bristles, where each of the hook-shaped bristles has a curved distal bristle tip (e.g., as illustrated in FIG. 15 and FIG. 16). In some aspects, each of the curved distal bristle tip of the hook-shaped bristles face, at least in part, in the proximal direction. In some aspects, the curved distal bristle tip of each of the hook-shaped bristles has a curvature of at least 90 degrees. In some aspects, the curved distal bristle tip of each of the hook-shaped bristles has a curvature of at least 135 degrees. In some aspects, the curved distal bristle tip of each of the hook-shaped bristles has a curvature of at least 180 degrees. In some aspects, the curved distal bristle tip of each of the hook-shaped bristles has a helical shape. In some aspects, the plurality of bristles includes bristles of a circular shape, a square shape, a rectangular shape, a polygon shape, a triangular shape, an oval shape, a trapezoid shape, a straight line longitudinally, a zigzag line longitudinally, or a bent line longitudinally.

At 3304, the method may include engaging the plurality of bristles with the material. In some aspects, to engage the plurality of bristles with the material, the medical device may be in the deployed configuration where the clot retrieval device (e.g., medical device) is no longer constrained within the lumen of the catheter and may be distal to the catheter. In some aspects, the material is trapped with the bristles or cavity of clot retrieval device, thus enabling the material to be removed from the target site. In some aspects, the material may be trapped between hook-shaped bristles such as 7G and 7F or 6G and 6F. In some aspects, the material may be trapped in a cavity, such as any particular cavity including cavity 174A, 174B, 202A, 202B, 218, 220, or 420A to 420R. In some aspects, the material may be trapped within any particular protrusion or groove such as 172, 173, 206A, 206B, 212, 252, 254, 424A to 424S, or 426A to 426S.

At 3306, the method may include retracting the balloon catheter, the plurality of bristles, and the material in a proximal direction. For example, an operator may pull the balloon catheter along the proximal direction to remove the material from a blood vessel while the balloon is inflated (e.g., FIG. 15). In some aspects, the blood vessel may be an artery (e.g., a brain artery). In some aspects, the balloon is inflated for at least a portion of the retracting. In some aspects, the balloon catheter is retracted towards a distal tip of a catheter. In some aspects, the catheter is an aspiration catheter. In some aspects, the balloon in an inflated state has an outer diameter that is substantially similar to an inner diameter of the blood vessel at the target site.

The method described in connection with FIG. 33 and FIG. 32 may be similarly performed with a clot-retrieval device with at least one coil, at least one cavity, or at least one semi-continuous protrusion or groove.

In some aspects, a medical device (e.g., 100, 600, 700, 800, 1100, 1200, 1500, 2500, or 3000), may include a pusher (e.g., 11, 111, 121, 151, or 221) and a clot-engaging device (e.g., 12, 62, 72, 82, 112, 122, 152, 222, or 273) coupled to the pusher, the clot-engaging device including a plurality of bristles (e.g., 1A-1S, 6A-6J, 7A-7K, 8A-8W, 20-28, 120A, 120B, 125A, 125B, 125C, 129A, 129B, 15A-15F, 234A-234J, 272A, 272B, 274A, or 274B) from a surface of the clot-engaging device.

In some aspects, each bristle of the plurality of bristles (e.g., 1A-1S, 6A-6J, 7A-7K, 8A-8W, 120A, 120B, 125A, 125B, 125C, 129A, 129B, 15A-15F, 234A-234J, 272A, 272B, 274A, or 274B) includes a first end attached to the outer surface of the clot-engaging device.

In some aspects, each bristle of the plurality of bristles (e.g., 6B-6C) further includes a second end attached to the outer surface of the clot-engaging device, each bristle of the plurality of bristles forming a loop with the surface.

In some aspects, the plurality of bristles includes a first set of bristles (e.g., at least one of 120A, 120B, 125A, 125B, 125C, 129A, 129B, 272A, or 274A) and a second set of bristles (e.g., 120A, 120B, 125A, 125B, 125C, 129A, 129B, 272A, or 274A), the second set of bristles being different than the first set of bristles. In some aspects, the first set of bristles and the second set of bristles differ by at least one of a body shape, a tip shape, a length, a width, or a material composition. In some aspects, the tip shape of each bristle in the first set of bristles is one of rounded, circular, square, rectangular, polygonal, triangular, helical, oval, or trapezoidal, and where the tip shape of each bristle in the second set of bristles is different from the first set of bristles. In some aspects, each bristle of the plurality of bristles further includes a tip shape that is one of rounded, circular, square, rectangular, polygonal, triangular, helical, oval, or trapezoidal. In some aspects, the body shape of each bristle in the first set of bristles is one of straight (e.g., 129A, 129B, 272A, or 274A), zigzagged (e.g., 120B), or bent (e.g., 125A or 125B), and where the body shape of each bristle in the second set of bristles is different from the first set of bristles. In some aspects, the material composition of each bristle in the first set of bristles is one of polymer, polymer-based, or nickel titanium. In some aspects, the material composition is polymer. In some aspects, the material composition is based on the polymer having a Shore hardness less than a particular value. In some aspects, the material composition is based on the polymer having a durometer less than a particular value.

In some aspects, the first set of bristles (e.g., a set that includes one of 234A-234J or a set that includes one of 1A-1S, 6A-6J, 7A-7K, 8A-8W, 120A, 120B, 125A, 125B, 125C, 129A, 129B, 15A-15F, or 234A-234J) is attached to a first cross-sectional portion of the outer surface of the clot-engaging device, and the second set of bristles (e.g., a set that includes a different one of 234A-234J or a set that includes a different one of 1A-1S, 6A-6J, 7A-7K, 8A-8W, 120A, 120B, 125A, 125B, 125C, 129A, 129B, 15A-15F, or 234A-234J) is attached to a second cross-sectional portion of the outer surface of the clot-engaging device, the first cross-sectional portion and the second cross-sectional portion being separated by a bristle-free region (e.g., a region that includes one of 232B-232J or an empty space between the other bristles) on the outer surface of the clot-engaging device.

In some aspects, the first set of bristles or the second set of bristles includes hook-shaped bristles, each of the hook-shaped bristles (e.g., 6A-6I or 7B-7H) having a curved distal bristle tip, the curved distal bristle tip pointing to one of a distal end (e.g., 64 or 74) of the clot-engaging device or a non-distal end (e.g., 63 or 73) of the clot-engaging device (e.g., 62 or 72).

In some aspects, where at least one of the first set of bristles or the second set of bristles includes only the hook-shaped bristles (e.g., 6A-6I or 7B-7H). In some aspects, the curved distal bristle tip of each of the hook-shaped bristles has a curvature of at least 90 degrees, 135 degrees, or 180 degrees. In some aspects, the curved distal bristle tip of each of the hook-shaped bristles has a helical shape. In some aspects, at least a portion of a body of each bristle of the plurality of bristles (e.g., 8A-8W, 7A, 7J, 15F, 234A-234J, 272A, 272B, 274A, or 274B) extends linearly in a radial direction from a center of the clot-engaging device. In some aspects, the portion of the body of each bristle of the plurality of bristles is coupled to the outer surface. In some aspects, at least one bristle of the plurality of bristles (e.g., 1A-1S or 1B1-1B8) is pointing in a tangential direction away from the outer surface of the clot-engaging device. In some aspects, the clot-engaging device (e.g., 82) is conically shaped.

In some aspects, the medical device may further include a catheter coupled to at least one of the clot-engaging device or the pusher, where, in a delivery configuration, the clot-engaging device is inside of the catheter, and where, in a deployed configuration, the clot-engaging device is outside of the catheter. In some aspects, in the deployed configuration, at least one of the plurality of bristles is configured to engage a clot. In some aspects, in the delivery configuration, at least one of the plurality of bristles is in a resting configuration, in the deployed configuration, at least one of the plurality of bristles is in a clot-engaging configuration different from the resting configuration.

In some aspects, the plurality of bristles (e.g., 15A-15F) extends from the outer surface of the clot-engaging device (e.g., 152) at a radius from an axial center of the clot-engaging device, the clot-engaging device further includes an inflatable balloon (e.g., 156) coupled to the pusher (e.g., 151), and the inflatable balloon is configured to change the radius of the plurality of bristles from the axial center of the clot-engaging device. In some aspects, the clot-engaging device further includes an intermediate layer (e.g., 157) exterior to the inflatable balloon (e.g., 156), the plurality of bristles (e.g., 15A-15F) is coupled to the intermediate layer, and the inflatable balloon is configured to change the radius of the intermediate layer from the axial center of the clot-engaging device. In some aspects, where the intermediate layer includes a flexible material that remains coupled to the inflatable balloon when the inflatable balloon expands from a deflated configuration (e.g., FIG. 15) to an inflated configuration (e.g., FIG. 16), where the radius is greater in the inflated configuration than in the deflated configuration. In some aspects, the medical device further includes a spring shaft (e.g., 155) located, at least in part, along an axial length of the inflatable balloon (e.g., 156). In some aspects, the spring shaft (e.g., 155) includes at least one coil. In some aspects, the spring shaft is coupled to the inflatable balloon at two or more axial locations (e.g., near 154 and 153) along the inflatable balloon.

In some aspects, the clot-engaging device (e.g., 222) further includes at least one coil (e.g., one of 232A-232J) located in a coil region of the clot-engaging device between a first bristle region (e.g., a region that includes one of 234A-234J) and a second bristle region (e.g., a region that includes another one of 234A-234J). In some aspects, the at least one coil is a non-metal material. In some aspects, the at least one coil is a polymer based material. In some aspects, the at least one coil includes a metal coil. In some aspects, each of the at least one coil (e.g., one of 232A-232J) is located in one of a plurality of coil regions on the surface, where each of the plurality of coil regions is wrapped around a circumference of the clot-engaging device, where each of the at least one coil is separated by a set of bristles (e.g., a set of bristles that includes one of 234A-234J) of the plurality of bristles. In some aspects, the clot-engaging device (e.g., 62, 82, 112) or the pusher (e.g., 11, 111) includes at least one radio-opaque marker (e.g., 66, 86, or 116).

In some aspects, a medical device may include a pusher (e.g., 11, 111, 121, 151, or 221) and a clot-engaging device (e.g., 1700 or 1900) coupled to the pusher, the clot-engaging device including at least one semi-continuous protrusion or groove (e.g., 172, 173, 206A, 206B, 212, 252, or 254) or at least one cavity (e.g., 174A, 174B, 202A, 202B, 218, 220, 420A to 420R) on an outer surface or an inner surface. In some aspects, each cavity of the at least one cavity (e.g., 174A, 174B, 202A, 202B, 218) has a cavity depth that is less than a thickness of the clot-engaging device. In some aspects, the at least one cavity (e.g., 174A) overlaps with the at least one semi-continuous protrusion or groove (e.g., 172). In some aspects, the at least one cavity (e.g., 174B, 202A, 202B, 218, 220, 420A to 420R) does not overlap with the at least one semi-continuous protrusion or groove (e.g., 172, 173, 206A, 206B). In some aspects, the at least one cavity (e.g., 174A, 202A, 202B, 218, 220, 420A to 420R) is located at the outer surface. In some aspects, the at least one cavity (e.g., 174B) is located at the inner surface. In some aspects, the at least one semi-continuous protrusion or groove (e.g., 172, 173, 206A, 206B) is located at the outer surface. In some aspects, the at least one semi-continuous protrusion or groove (e.g., 212) is located at the inner surface. In some aspects, the at least one semi-continuous protrusion or groove (e.g., 172 or 173) forms a plurality of kite-shaped regions (e.g., 176A or 176B, 422A to 422R) on the outer surface of the clot-engaging device, and where each cavity of the at least one cavity (e.g., 174B) is located within the plurality of kite-shaped regions. In some aspects, the at least one cavity (e.g., 174B) is substantially centered within the plurality of kite-shaped regions (e.g., 176B). In some aspects, the at least one semi-continuous protrusion or groove is based on a sweep cutout.

In some aspects, the at least one semi-continuous protrusion or groove has a helical pattern (e.g., 172, 173, 206A, 206B, 212, 252, 254, 424A to 424S, 426A to 426S). In some aspects, the at least one semi-continuous protrusion or groove includes a plurality of protrusions or grooves (e.g., 172, 173, 206A, 206B, 212, 252, 254, 424A to 424S, 426A to 426S) along the outer surface. In some aspects, each of the plurality of protrusions or grooves each has a helical pattern.

In some aspects, the plurality of protrusions or grooves includes one or more pairs of protrusions or grooves (e.g., 252/254, 206A/206B, 424A/426A, or 172/173), where each of the one or more pairs of protrusions or grooves include a first semi-continuous protrusion or groove (e.g., one of 252/254, one of 206A/206B, one of 424A/426A, or one of 172/173) having a clockwise helical pattern and a second semi-continuous protrusion or groove (e.g., another one of 252/254, another one of 206A/206B, another one of 424A/426A, or another one of 172/173) having a counterclockwise helical pattern. In some aspects, the one or more pairs of protrusions or grooves include at least two pairs of protrusions or grooves. In some aspects, the one or more pairs of protrusions or grooves include at least three pairs of protrusions or grooves. In some aspects, the protrusions having the clockwise helical pattern (e.g., different segments of 173) are spaced substantially equidistant from one another on the outer surface of the clot-engaging device, or where the protrusions (e.g., different segments of 172) having the counterclockwise helical pattern are spaced substantially equidistant from one another on the outer surface of the clot-engaging device.

In some aspects, the plurality of protrusions or grooves includes at least a first semi-continuous groove (e.g., 172) having a first depth and a second semi-continuous groove (e.g., 173) having a second depth, where the first depth and the second depth are both less than a thickness of the clot-engaging device, and where the first depth and the second depth are unequal to each other.

In some aspects, a depth of the at least one semi-continuous protrusion or groove (e.g., 172 or 173) is a variable depth. In some aspects, the clot-engaging device (e.g., 1700, 1900) or the pusher includes at least one radio-opaque marker (e.g., 66, 86, or 116).

In some aspects, the medical device further includes an inflatable balloon (e.g., 156) coupled to the pusher (e.g., 151), where the at least one semi-continuous protrusion or groove or the at least one cavity is located at the outer surface, and where the clot-engaging device is exterior to the inflatable balloon. In some aspects, the at least one semi-continuous protrusion or groove or the at least one cavity is coupled to the inflatable balloon by an intermediate layer (e.g., 157), where the at least one semi-continuous protrusion or groove or the at least one cavity is coupled to an exterior surface of the intermediate layer, and the inflatable balloon is coupled to an interior surface of the intermediate layer. In some aspects, the intermediate layer includes a flexible material that remains coupled to the inflatable balloon when the inflatable balloon expands from a deflated configuration (e.g., FIG. 15) to an inflated configuration (e.g., FIG. 16), where the radius is greater in the inflated configuration than in the deflated configuration. In some aspects, the clot-engaging device further includes a spring shaft (e.g., 155) located, at least in part, along an axial length of the inflatable balloon. In some aspects, the spring shaft includes at least one coil. In some aspects, the spring shaft is coupled to the inflatable balloon at two or more axial locations along the balloon catheter. In some aspects, the medical device further includes a catheter coupled to at least one of the clot-engaging device or the pusher, where, in a delivery configuration, the clot-engaging device is inside of the catheter, and where, in a deployed configuration, the clot-engaging device is outside of the catheter.

In some aspects, the elongated member (e.g., 122, 1700) has at least one semi-continuous protrusion or groove or at least one cavity (e.g., 127A, 127B, 174A, or 174B) along an outer surface or an inner surface of the elongated member with a depth that is less than a thickness of the elongated member. In some aspects, the elongated member has at least one semi-continuous protrusion or groove or along an outer surface or an inner surface of the elongated member with a depth that is less than a thickness of the elongated member at least one cavity along the outer surface or the inner surface of the elongated member. In some aspects, each cavity of the at least one cavity has a cavity depth that is less than the thickness of the elongated member. In some aspects, each cavity of the at least one cavity has a cavity depth that is less than the thickness of the elongated member.

In some aspects, each cavity of the at least one cavity (e.g., 174A, 174B, 202A, 202B, 218) has a cavity depth that is less than a thickness of the clot-engaging device. In some aspects, the at least one cavity (e.g., 174A) overlaps with the at least one semi-continuous protrusion or groove (e.g., 172). In some aspects, the at least one cavity (e.g., 174B, 202A, 202B, 218, 220, 420A to 420R) does not overlap with the at least one semi-continuous protrusion or groove (e.g., 172, 173, 206A, 206B). In some aspects, the at least one cavity (e.g., 174A, 202A, 202B, 218, 220, 420A to 420R) is located at the outer surface. In some aspects, the at least one cavity (e.g., 174B) is located at the inner surface. In some aspects, the at least one semi-continuous protrusion or groove (e.g., 172, 173, 206A, 206B) is located at the outer surface. In some aspects, the at least one semi-continuous protrusion or groove (e.g., 212) is located at the inner surface. In some aspects, the at least one semi-continuous protrusion or groove (e.g., 172 or 173) forms a plurality of kite-shaped regions (e.g., 176A or 176B, 422A to 422R) on the outer surface of the clot-engaging device, and where each cavity of the at least one cavity (e.g., 174B) is located within the plurality of kite-shaped regions. In some aspects, the at least one cavity (e.g., 174B) is substantially centered within the plurality of kite-shaped regions (e.g., 176B). In some aspects, the at least one semi-continuous protrusion or groove is based on a sweep cutout.

In some aspects, the at least one semi-continuous protrusion or groove has a helical pattern (e.g., 172, 173, 206A, 206B, 212, 252, 254, 424A to 424S, 426A to 426S). In some aspects, the at least one semi-continuous protrusion or groove includes a plurality of protrusions or grooves (e.g., 172, 173, 206A, 206B, 212, 252, 254, 424A to 424S, 426A to 426S) along the outer surface. In some aspects, each of the plurality of protrusions or grooves each has a helical pattern.

In some aspects, the plurality of protrusions or grooves includes one or more pairs of protrusions or grooves (e.g., 252/254, 206A/206B, or 172/173), where each of the one or more pairs of protrusions or grooves include a first semi-continuous protrusion or groove (e.g., one of 252/254, one of 206A/206B, or one of 172/173) having a clockwise helical pattern and a second semi-continuous protrusion or groove (e.g., another one of 252/254, another one of 206A/206B, or another one of 172/173) having a counterclockwise helical pattern. In some aspects, the one or more pairs of protrusions or grooves include at least two pairs of protrusions or grooves. In some aspects, the one or more pairs of protrusions or grooves include at least three pairs of protrusions or grooves. In some aspects, the protrusions having the clockwise helical pattern (e.g., different segments of 173) are spaced substantially equidistant from one another on the outer surface of the clot-engaging device, or where the protrusions (e.g., different segments of 172) having the counterclockwise helical pattern are spaced substantially equidistant from one another on the outer surface of the clot-engaging device.

In some aspects, the plurality of protrusions or grooves includes at least a first semi-continuous groove (e.g., 172) having a first depth and a second semi-continuous groove (e.g., 173) having a second depth, where the first depth and the second depth are both less than a thickness of the clot-engaging device, and where the first depth and the second depth are unequal to each other.

In some aspects, a depth of the at least one semi-continuous protrusion or groove (e.g., 172 or 173) is a variable depth. In some aspects, the clot-engaging device (e.g., 1700, 1900) or the pusher includes at least one radio-opaque marker (e.g., 66, 86, or 116).

In some aspects, a medical device (e.g., 100, 600, 700, 800, 1100, 1200, 1500, 2500, or 3000) may include a pusher (e.g., 11, 111, 121, 151, or 221) with a distal end having an elongated member (e.g., 12, 62, 72, 82, 112, 122, 152, 222, or 273); a plurality of bristles (e.g., 1A-1S, 6A-6J, 7A-7K, 8A-8W, 20-28, 120A, 120B, 125A, 125B, 125C, 129A, 129B, 15A-15F, 234A-234J, 272A, 272B, 274A, or 274B), where each bristle has a proximal bristle end distributed along the elongated member and a distal bristle tip not distributed along the elongated member, and where the plurality of bristles includes: a first set of bristles (e.g., a set that includes one of 1A-1S, 6A-6J, 7A-7K, 8A-8W, 120A, 120B, 125A, 125B, 125C, 129A, 129B, 15A-15F, 234A-234J, 272A, 272B, 274A, or 274B) distributed along a first cross-sectional portion of the elongated member, and a second set of bristles (e.g., a different set that includes a different one of 1A-1S, 6A-6J, 7A-7K, 8A-8W, 120A, 120B, 125A, 125B, 125C, 129A, 129B, 15A-15F, 234A-234J, 272A, 272B, 274A, or 274B) distributed along a second cross-sectional portion of the elongated member; and where the elongated member has a first bristle-free region (e.g., a region that includes one of 232B-232J or an empty space between the other bristles) located between the first cross-sectional portion and the second cross-sectional portion of the elongated member. In some aspects, in a delivery configuration, the medical device is within a lumen of a catheter, and where, in a deployed configuration, the medical device is distal to the catheter. In some aspects, each bristle of the plurality of bristles has a rounded tip and the elongated member is polymer-based. In some aspects, the first set of bristles (e.g., a set that includes one of 1A-1S, 6A-6J, 7A-7K, 8A-8W, 15A-15F, 234A-234J, 272A, 272B, 274A, or 274B) is parallel to the second set of bristles (e.g., a different set that includes a different one of 1A-1S, 6A-6J, 7A-7K, 8A-8W, 15A-15F, 234A-234J, 272A, 272B, 274A, or 274B).

In some aspects, a medical device (e.g., 100, 600, 700, 800, 1100, 1200, 1500, 2500, or 3000) may include a pusher (e.g., 11, 111, 121, 151, or 221) with a distal end having an elongated member (e.g., 12, 62, 72, 82, 112, 122, 152, 222, or 273); a plurality of bristles (e.g., 1A-1S, 6A-6J, 7A-7K, 8A-8W, 20-28, 120A, 120B, 125A, 125B, 125C, 129A, 129B, 15A-15F, 234A-234J, 272A, 272B, 274A, or 274B), where each bristle has a proximal bristle end distributed along the elongated member and a distal bristle tip not distributed along the elongated member, and where the plurality of bristles includes: a first set of bristles (e.g., a set that includes one of 1A-1S, 6A-6J, 7A-7K, 8A-8W, 120A, 120B, 125A, 125B, 125C, 129A, 129B, 15A-15F, 234A-234J, 272A, 272B, 274A, or 274B) distributed along a first longitudinal portion of the elongated member, and a second set of bristles (e.g., a different set that includes a different one of 1A-1S, 6A-6J, 7A-7K, 8A-8W, 120A, 120B, 125A, 125B, 125C, 129A, 129B, 15A-15F, 234A-234J, 272A, 272B, 274A, or 274B) distributed along a second longitudinal portion of the elongated member; and where the elongated member has a first bristle-free region (e.g., a region that includes one of 232B-232J or an empty space between the other bristles) located between the first longitudinal portion and the second longitudinal portion of the elongated member. In some aspects, in a delivery configuration, the medical device is within a lumen of a catheter, and where, in a deployed configuration, the medical device is distal to the catheter. In some aspects, each bristle of the plurality of bristles has a rounded tip and the elongated member is polymer-based. In some aspects, the first set of bristles (e.g., a set that includes one of 1A-1S, 6A-6J, 7A-7K, 8A-8W, 15A-15F, 234A-234J, 272A, 272B, 274A, or 274B) is parallel to the second set of bristles (e.g., a different set that includes a different one of 1A-1S, 6A-6J, 7A-7K, 8A-8W, 15A-15F, 234A-234J, 272A, 272B, 274A, or 274B).

In some aspects, each bristle of the plurality of bristles (e.g., 8A-8W, 234A-234J, 272A, 272B, 274A, or 274B) has a straight bristle portion that is aligned in a radial direction extending outwardly from a center of the elongated member. In some aspects, the straight bristle portion of each bristle of the plurality of bristles is located at least in part at the proximal bristle end of the bristle.

In some aspects, at least one of the first set of bristles or the second set of bristles include hook-shaped bristles (e.g., 6A-6I or 7B-7H), where each of the hook-shaped bristles has a curved distal bristle tip, the curved distal bristle tip pointing to the distal end (e.g., 64, 74) or a non-distal end (e.g., 63, 73). In some aspects, at least one of the first set of bristles or the second set of bristles includes only the hook-shaped bristles (e.g., 6A-6I or 7B-7H), where each of the hook-shaped bristles has the curved distal bristle tip. In some aspects, the curved distal bristle tip of each of the hook-shaped bristles has a curvature of at least 90 degrees. In some aspects, the curved distal bristle tip of each of the hook-shaped bristles has a curvature of at least 135 degrees. In some aspects, the curved distal bristle tip of each of the hook-shaped bristles has a curvature of at least 180 degrees. In some aspects, the curved distal bristle tip of each of the hook-shaped bristles has a helical shape. In some aspects, each bristle of the plurality of bristles is a hook-shaped bristles where each of the hook-shaped bristles has a curved distal bristle tip, the curved distal bristle tip pointing to the distal end (e.g., 64, 74) or a non-distal end (e.g., 63, 73). In some aspects, at least one of the first set of bristles or the second set of bristles includes bristles of a circular shape, a square shape, a rectangular shape, a polygon shape, a triangular shape, an oval shape, a trapezoid shape, a straight line longitudinally, a zigzag line longitudinally (e.g., 120B), or a bent line longitudinally (e.g., 125A, 125B). In some aspects, the first cross-sectional portion and the second cross-sectional portion are located at a tapered region of the elongated member (e.g., 82), where the first cross-sectional portion of the elongated member has a first diameter, where the second cross-sectional portion of the elongated member has a second diameter, and where the first diameter is larger than the second diameter.

In some aspects, each bristle in the first set of bristles (e.g., a different one of 8A-8W) has a first length, where each bristle in the second set of bristles has a second length, and where the first length equals the second length. In some aspects, each bristle in the first set of bristles (e.g., one of 8A-8W or 274A/274B) has a first length, where each bristle in the second set of bristles (e.g., a different one of 8A-8W or 272A/272B) has a second length, and where the first length is greater than the second length. In some aspects, each bristle in the first set of bristles (e.g., a different one of 8A-8W or 272A/272B) has a first length, where each bristle in the second set of bristles (e.g., one of 8A-8W or 274A/274B) has a second length, and where the first length is less than the second length. In some aspects, a first subset of bristles in the first set of bristles and a second subset of bristles in the second set of bristles has a first length, where a third subset of bristles in the first set of bristles and a fourth subset of bristles in the second set of bristles has a second length, and where the first length is less than the second length.

In some aspects, at least one bristle of the plurality of bristles (e.g., 1A-1S) is pointing in a tangential direction away from a circle associated with the elongated member (e.g., 12).

In some aspects, the first set of bristles are distributed along a circumference of the elongated member based on a pattern. In some aspects, the pattern is based on the first set of bristles being associated with a same axial distance associated with the elongated member. In some aspects, the pattern is based on the first set of bristles being associated with a particular range of axial distance of the elongated member. In some aspects, the elongated member (e.g., FIG. 4) is hollow. In some aspects, the plurality of bristles (e.g., 20-28) is distributed along an inner surface of the elongated member. In some aspects, the elongated member is a hollow tube (e.g., FIG. 4). In some aspects, the elongated member is non-hollow (e.g., 12, 62, 72, 82, 112, 122, 152, 222, or 273).

In some aspects, the elongated member is cylindrical (e.g., 82). In some aspects, the plurality of bristles (e.g., 1A-1S, 6A-6J, 7A-7K, 8A-8W, 120A, 120B, 125A, 125B, 125C, 129A, 129B, 15A-15F, 234A-234J, 272A, 272B, 274A, or 274B) is distributed along an outer surface of the elongated member.

In some aspects, the first set of bristles is located at a first bristle region (e.g., a region that includes one of 234A-234J) of the elongated member and the second set of bristles located at a second bristle region (e.g., a region that includes another one of 234A-234J) of the elongated member, and where the first bristle region and the second bristle region are spaced apart by a distance along the elongated member. In some aspects, the first bristle region is located at the first cross-sectional portion of the elongated member, where the second bristle region is located at the second cross-sectional portion of the elongated member, and where the first cross-sectional portion and the second cross-sectional portion are spaced apart on the elongated member by a longitudinal distance. In some aspects, the first bristle region is located at a first cross-section of the elongated member, where the first bristle region is located at the first cross-sectional portion of the elongated member, where the second bristle region is located at the second cross-sectional portion of the elongated member, and where the first cross-sectional portion and the second cross-sectional portion are spaced apart at different circumferential regions of the elongated member.

In some aspects, the elongated member further includes at least one coil (e.g., one of 232A-232J) located in a coil region of the elongated member between a first bristle region (e.g., a region that includes one of 234A-234J) and a second bristle region (e.g., a region that includes another one of 234A-234J).

In some aspects, the at least one coil is a non-metal material. In some aspects, the at least one coil is a polymer based material. In some aspects, the at least one coil includes a metal coil. In some aspects, each of the at least one coil (e.g., one of 232A-232J) is located in one of a plurality of coil regions on the surface, where each of the plurality of coil regions is wrapped around a circumference of the elongated member, where each of the at least one coil is separated by a set of bristles (e.g., a set of bristles that includes one of 234A-234J) of the plurality of bristles. In some aspects, the first set of bristles include a first plurality of bristles (e.g., 272A, 272B) having a substantially uniform first length. In some aspects, the second set of bristles include a second plurality of bristles (e.g., 274A, or 274B) having a substantially uniform second length. In some aspects, the substantially uniform first length is less than the substantially uniform second length.

In some aspects, the plurality of bristles (e.g., 15A-15F) extends from the outer surface of the elongated member (e.g., 152) at a radius from an axial center of the elongated member, the elongated member further includes an inflatable balloon (e.g., 156) coupled to the pusher (e.g., 151), and the inflatable balloon is configured to change the radius of the plurality of bristles from the axial center of the elongated member. In some aspects, the elongated member further includes an intermediate layer (e.g., 157) exterior to the inflatable balloon (e.g., 156), the plurality of bristles (e.g., 15A-15F) is coupled to the intermediate layer, and the inflatable balloon is configured to change the radius of the intermediate layer from the axial center of the elongated member. In some aspects, where the intermediate layer includes a flexible material that remains coupled to the inflatable balloon when the inflatable balloon expands from a deflated configuration (e.g., FIG. 15) to an inflated configuration (e.g., FIG. 16), where the radius is greater in the inflated configuration than in the deflated configuration. In some aspects, the medical device further includes a spring shaft (e.g., 155) located, at least in part, along an axial length of the inflatable balloon (e.g., 156). In some aspects, the spring shaft (e.g., 155) includes at least one coil. In some aspects, the spring shaft is coupled to the inflatable balloon at two or more axial locations (e.g., near 154 and 153) along the inflatable balloon.

In some aspects, the elongated member has at least one semi-continuous protrusion or groove (e.g., 172, 173, 206A, 206B, 212, 252, 254, 424A to 424S, 426A to 426S) or at least one cavity (e.g., 174A, 174B, 202A, 202B, 218, 220, 420A to 420R) along an outer surface or an inner surface of the elongated member with a depth that is less than a thickness of the elongated member.

In some aspects, each cavity of the at least one cavity (e.g., 174A, 174B, 202A, 202B, 218) has a cavity depth that is less than a thickness of the elongated member. In some aspects, the at least one cavity (e.g., 174A) overlaps with the at least one semi-continuous protrusion or groove (e.g., 172). In some aspects, the at least one cavity (e.g., 174B, 202A, 202B, 218, 220, 420A to 420R) does not overlap with the at least one semi-continuous protrusion or groove (e.g., 172, 173, 206A, 206B). In some aspects, the at least one cavity (e.g., 174A, 202A, 202B, 218, 220, 420A to 420R) is located at the outer surface. In some aspects, the at least one cavity (e.g., 174B) is located at the inner surface. In some aspects, the at least one semi-continuous protrusion or groove (e.g., 172, 173, 206A, 206B) is located at the outer surface. In some aspects, the at least one semi-continuous protrusion or groove (e.g., 212) is located at the inner surface. In some aspects, the at least one semi-continuous protrusion or groove (e.g., 172 or 173) forms a plurality of kite-shaped regions (e.g., 176A or 176B, 422A to 422R) on the outer surface of the elongated member, and where each cavity of the at least one cavity (e.g., 174B) is located within the plurality of kite-shaped regions. In some aspects, the at least one cavity (e.g., 174B) is substantially centered within the plurality of kite-shaped regions (e.g., 176B). In some aspects, the at least one semi-continuous protrusion or groove is based on a sweep cutout.

In some aspects, the at least one semi-continuous protrusion or groove has a helical pattern (e.g., 172, 173, 206A, 206B, 212, 252, 254, 424A to 424S, 426A to 426S). In some aspects, the at least one semi-continuous protrusion or groove includes a plurality of protrusions or grooves (e.g., 172, 173, 206A, 206B, 212, 252, 254, 424A to 424S, 426A to 426S) along the outer surface. In some aspects, each of the plurality of protrusions or grooves each has a helical pattern.

In some aspects, the plurality of protrusions or grooves includes one or more pairs of protrusions or grooves (e.g., 252/254, 206A/206B, or 172/173), where each of the one or more pairs of protrusions or grooves include a first semi-continuous protrusion or groove (e.g., one of 252/254, one of 206A/206B, or one of 172/173) having a clockwise helical pattern and a second semi-continuous protrusion or groove (e.g., another one of 252/254, another one of 206A/206B, or another one of 172/173) having a counterclockwise helical pattern. In some aspects, the one or more pairs of protrusions or grooves include at least two pairs of protrusions or grooves. In some aspects, the one or more pairs of protrusions or grooves include at least three pairs of protrusions or grooves. In some aspects, the protrusions having the clockwise helical pattern (e.g., different segments of 173) are spaced substantially equidistant from one another on the outer surface of the elongated member, or where the protrusions (e.g., different segments of 172) having the counterclockwise helical pattern are spaced substantially equidistant from one another on the outer surface of the elongated member.

In some aspects, the plurality of protrusions or grooves includes at least a first semi-continuous groove (e.g., 172) having a first depth and a second semi-continuous groove (e.g., 173) having a second depth, where the first depth and the second depth are both less than a thickness of the elongated member, and where the first depth and the second depth are unequal to each other. In some aspects, the plurality of cavities may include a first cavity (e.g., 174A) having a first volume, a second cavity (e.g., 174B) having a second volume, and where the first volume and second volume are different or the same.

In some aspects, a depth of the at least one semi-continuous protrusion or groove (e.g., 172 or 173) is a variable depth. In some aspects, the elongated member (e.g., 1700, 1900) or the pusher includes at least one radio-opaque marker (e.g., 66, 86, or 116). In some aspects, the elongated member (e.g., 82) is conically shaped. In some aspects, the at least one semi-continuous protrusion or groove or the at least one cavity is located at the outer surface, and where the elongated member (e.g., 152) further includes an inflatable balloon (e.g., 154).

In some aspects, a medical device may include an elongated member (e.g., 1700, 1900, or 2800) having an outer surface, inner lumen, and a tube body therebetween, where the elongated member has at least one semi-continuous protrusion or groove (e.g., 172, 173, 206A, 206B, 212, 252, 254, 424A to 424S, 426A to 426S) along the outer surface or an inner surface of the elongated member with a depth that is less than a thickness of the elongated member. In some aspects, the plurality of cavities is an array of cavities spaced apart from one another.

In some aspects, the plurality of cavities is an array of cavities (e.g., including 174A/174B or 202A/202B) spaced apart from one another. In some aspects, the plurality of cavities includes a first cavity (e.g., 174A) having a first volume, a second cavity (e.g., 174B) having a second volume, and where the first volume and second volume are different. In some aspects, the first cavity (e.g., 174A) has a first depth, the second cavity (e.g., 174B) has a second depth, and where the first depth and second depth are different. In some aspects, the first cavity has a first diameter at the outer surface of the elongated member, the second cavity has a second diameter at the outer surface of the elongated member, and the first diameter and the second diameter are different. In some aspect, in a collection configuration a first foreign body is at least partially within the first cavity and a second foreign body is at least partially within the second cavity. In some aspects, the first foreign body and the second foreign body are blood clots of different sizes. In some aspects, the medical device may further include a catheter having an inner catheter lumen, where in the collection configuration the first foreign body and the second foreign body are trapped between the plurality of cavities and the inner catheter lumen. In some aspects, the plurality of cavities (e.g., including cavity 202A or 202B) includes at least four (e.g., twelve cavities including cavity 202A and 202B) cavities spaced apart equally along the outer surface of the elongated member (e.g., 1900). In some aspects, in a collection configuration a first foreign body is at least partially within at least one semi-continuous protrusion or groove. In some aspects, the first foreign body is a blood clot. In some aspects, the medical device includes a catheter having an inner catheter lumen, where in the collection configuration the first foreign body is trapped between the at least one semi-continuous protrusion or groove and the inner catheter lumen.

In some aspects, a medical device may include an elongated member (e.g., 1700, 1900, or 2800) having an outer surface, inner lumen, and a tube body therebetween, and at least one helical protrusion or groove (e.g., 252, 254, 172, 173) that spirals helically and longitudinally along the outer surface of the elongated member for one or more rotations. In some aspects, the at least one helical protrusion or groove includes a first helical protrusion or groove (e.g., one of 252 or 254, one of 172 or 173, 426A to 426S) oriented in a clockwise direction and a second helical protrusion or groove (e.g., one of 252 or 254 or one of 172 or 173, 424A to 424S) oriented in a counterclockwise direction. In some aspects, the elongated member is tapered near a distal end of the elongated member. In some aspects, the elongated member includes at least one cavity (e.g., 202A or 202B, 420A to 420R) that extends radially outward from the elongated member to the outer surface. In some aspects, the at least one cavity has a depth that is less than a thickness of the elongated member of the elongated member. In some aspects, the at least one cavity (e.g., 174A or 174B) is disposed at a region along the outer surface formed by intersections of the least one helical protrusion or groove (e.g., 172 or 173). In some aspects, in a retrieval configuration, a portion of the at least one helical protrusion or groove or the at least one cavity is engaged with a clot fragment. In some aspects, in an unconstrained configuration, the medical device is within a catheter, and navigated distally along a vessel to a region proximate to a clot. In some aspects, the medical device may include a plurality of cavities (e.g., 202A, 202B, 420A to 420R, and the like) on the outer surface of the elongated member, where each of the plurality of cavities has a cavity depth that is less than a thickness of the elongated member. In some aspects, at least one of the plurality of cavities overlaps with at least one semi-continuous protrusion or groove. In some aspects, the at least one cavity (e.g., 174A) overlaps with the at least one semi-continuous protrusion or groove (e.g., 172). In some aspects, the at least one cavity (e.g., 174B, 202A, 202B, 218, 220, 420A to 420R) does not overlap with the at least one semi-continuous protrusion or groove (e.g., 172, 173, 206A, 206B). In some aspects, the plurality of cavities (e.g., 218, 220, 420A to 420R) is spaced apart from the at least one semi-continuous protrusion or groove (e.g., 206A, 206B, 424A to 424S, 426A to 426S) on the outer surface of the elongated member. In some aspects, the plurality of cavities (e.g., 202A, 202B, 420A to 420R) is an array of cavities spaced apart from one another. In some aspects, the at least one semi-continuous protrusion or groove (e.g., 172 or 173) forms a plurality of kite-shaped regions (e.g., 176A or 176B, 422A to 422R) on the outer surface of the elongated member, and where each cavity of the at least one cavity (e.g., 174B, 420A to 420R) is located within the plurality of kite-shaped regions. In some aspects, the at least one cavity (e.g., 174B, 420A to 420R) is substantially centered within the plurality of kite-shaped regions (e.g., 176B, 422A to 422R).

In some aspects, a medical device may include a balloon catheter (e.g., 1300, 1500) having a proximal region, a distal region, and an internal lumen therebetween; an inflatable balloon (e.g., 132, 154) coupled to the distal region of the balloon catheter, where the inflatable balloon is in fluid communication with an inflation port at the proximal region of the balloon catheter via an inflation channel (e.g., near 154) within the balloon catheter; and a plurality of bristles (e.g., 15A-15F) distributed along the inflatable balloon or a spring shaft (e.g., 155) located, at least in part, along an axial length of the balloon catheter that includes the inflatable balloon.

In some aspects, the distal region of the balloon catheter further includes an intermediate layer (e.g., 157), where the plurality of bristles is coupled to the intermediate layer, and the inflatable balloon is configured to change a radius of the intermediate layer from an axial center of the balloon catheter. In some aspects, where the intermediate layer includes a flexible material that remains coupled to the inflatable balloon when the inflatable balloon expands from a deflated configuration (e.g., FIG. 15) to an inflated configuration (e.g., FIG. 16), where the radius is greater in the inflated configuration than in the deflated configuration.

In some aspects, a method of removing a material from a blood vessel may include navigating a catheter (e.g., 100, 600, 700, 800, 1100, 1200, 1500, 2500) into the blood vessel to a target site that is distal to the material, where the catheter includes: a pusher (e.g., 11, 111, 121, 151, or 221) with a distal end having an elongated member (e.g., 12, 62, 72, 82, 112, 122, 152, 222, or 273), a plurality of bristles (e.g., 1A-1S, 6A-6J, 7A-7K, 8A-8W, 20-28, 120A, 120B, 125A, 125B, 125C, 129A, 129B, 15A-15F, 234A-234J, 272A, 272B, 274A, or 274B), where each bristle has a proximal bristle end coupled to the elongated member and a distal bristle tip not coupled to the elongated member, and where the plurality of bristles includes: a first set of bristles (e.g., a set that includes one of 1A-1S, 6A-6J, 7A-7K, 8A-8W, 120A, 120B, 125A, 125B, 125C, 129A, 129B, 15A-15F, 234A-234J, 272A, 272B, 274A, or 274B) coupled to a first longitudinal portion of the elongated member, and a second set of bristles e.g., a different set that includes a different one of 1A-1S, 6A-6J, 7A-7K, 8A-8W, 120A, 120B, 125A, 125B, 125C, 129A, 129B, 15A-15F, 234A-234J, 272A, 272B, 274A, or 274B) coupled to a second longitudinal portion of the elongated member; and where the elongated member has a first bristle-free region (e.g., a region that includes one of 232B-232J or an empty space between the other bristles) located between the first longitudinal portion and the second longitudinal portion of the elongated member; engaging the plurality of bristles with the material; retracting the catheter, the plurality of bristles, and the material in a proximal direction.

In some aspects, a method of removing a material from a blood vessel may include navigating a catheter (e.g., 100, 600, 700, 800, 1100, 1200, 1500, 2500) into the blood vessel to a target site that is distal to the material, where the catheter includes: a pusher (e.g., 11, 111, 121, 151, or 221) with a distal end having an elongated member (e.g., 12, 62, 72, 82, 112, 122, 152, 222, or 273), a plurality of bristles (e.g., 1A-1S, 6A-6J, 7A-7K, 8A-8W, 20-28, 120A, 120B, 125A, 125B, 125C, 129A, 129B, 15A-15F, 234A-234J, 272A, 272B, 274A, or 274B), where each bristle has a proximal bristle end coupled to the elongated member and a distal bristle tip not coupled to the elongated member, and where the plurality of bristles includes: a first set of bristles (e.g., a set that includes one of 1A-1S, 6A-6J, 7A-7K, 8A-8W, 120A, 120B, 125A, 125B, 125C, 129A, 129B, 15A-15F, 234A-234J, 272A, 272B, 274A, or 274B) coupled to a first cross-sectional portion of the elongated member, and a second set of bristles e.g., a different set that includes a different one of 1A-1S, 6A-6J, 7A-7K, 8A-8W, 120A, 120B, 125A, 125B, 125C, 129A, 129B, 15A-15F, 234A-234J, 272A, 272B, 274A, or 274B) coupled to a second cross-sectional portion of the elongated member; and where the elongated member has a first bristle-free region (e.g., a region that includes one of 232B-232J or an empty space between the other bristles) located between the first cross-sectional portion and the second cross-sectional portion of the elongated member; engaging the plurality of bristles with the material; retracting the catheter, the plurality of bristles, and the material in a proximal direction.

In some aspects, a method of removing a material from a blood vessel includes navigating a balloon catheter (e.g., 1500) into the blood vessel to a target site that is distal to the material, where the balloon catheter includes an inflatable balloon (e.g., 156) having an outer surface with a plurality of bristles (e.g., 15A-15F); inflating the balloon; engaging the plurality of bristles with the material; retracting the balloon catheter, the plurality of bristles, and the material in a proximal direction. In some aspects, the inflatable balloon is inflated for at least a portion of the retracting. In some aspects, the inflatable balloon catheter is retracted towards a distal tip of a catheter. In some aspects, the catheter is an aspiration catheter. In some aspects, the inflatable balloon in an inflated state has an outer diameter that is substantially similar to an inner diameter of the blood vessel at the target site. In some aspects, each bristle of the plurality of bristles includes at least one hook-shaped bristle (e.g., 15A-15F), each of the at least one hook-shaped bristle has a curved distal bristle tip. In some aspects, each of the curved distal bristle tip of the hook-shaped bristles face, at least in part, in the proximal direction. In some aspects, the curved distal bristle tip of each of the at least one hook-shaped bristle has a curvature of at least 90 degrees. In some aspects, the curved distal bristle tip of each of the at least one hook-shaped bristle has a curvature of at least 135 degrees. In some aspects, the curved distal bristle tip of each of the at least one hook-shaped bristle has a curvature of at least 180 degrees. In some aspects, the curved distal bristle tip of each of the at least one hook-shaped bristle has a helical shape. In some aspects, the plurality of bristles includes bristles of a circular shape, a square shape, a rectangular shape, a polygon shape, a triangular shape, an oval shape, a trapezoid shape, a straight line longitudinally, a zigzag line longitudinally, or a bent line longitudinally.

In some aspects, a medical device (e.g., 100, 600, 700, 800, 1100, 1200, 1500, 2500, or 3000) may include a pusher (e.g., 11, 111, 121, 151, or 221) with a distal end having an elongated member (e.g., 12, 62, 72, 82, 112, 122, 152, 222, or 273); and a set of bristle means (e.g., 1A-1S, 6A-6J, 7A-7K, 8A-8W, 20-28, 120A, 120B, 125A, 125B, 125C, 129A, 129B, 15A-15F, 234A-234J, 272A, 272B, 274A, or 274B) for capturing a foreign body and coupled to the elongated member.

In some aspects, a medical device may include a pusher (e.g., 11, 111, 121, 151, or 221) with a distal end having an elongated member (e.g., 1700 or 1900); and a set of cavity means (e.g., 174A, 174B, 202A, 202B, 218, 220, 420A to 420R) or a set of semi-continuous protrusion or groove means (e.g., 172, 173, 206A, 206B, 212, 252, or 254) for capturing a foreign body and coupled to the elongated member.

In some aspects, a medical device (e.g., 1500) may include a pusher with a distal end having an elongated member (e.g., 152); and at least one capturing means (e.g., 15A-15F) for capturing a foreign body and distributed along an inflatable means (e.g., 156) associated with the elongated member.

It is understood that the specific order or hierarchy of blocks in the processes/flowcharts disclosed is an illustration of example approaches. Based upon design preferences, it is understood that the specific order or hierarchy of blocks in the processes/flowcharts may be rearranged. Further, some blocks may be combined or omitted. The accompanying method claims present elements of the various blocks in a sample order, and are not limited to the specific order or hierarchy presented.

The previous description is provided to enable any person skilled in the art to practice the various aspects described herein. Various modifications to these aspects will be readily apparent to those skilled in the art, and the generic principles defined herein may be applied to other aspects. Thus, the claims are not limited to the aspects described herein, but are to be accorded the full scope consistent with the language claims. Reference to an element in the singular does not mean “one and only one” unless specifically so stated, but rather “one or more.” Terms such as “if,” “when,” and “while” do not imply an immediate temporal relationship or reaction. That is, these phrases, e.g., “when,” do not imply an immediate action in response to or during the occurrence of an action, but simply imply that if a condition is met then an action will occur, but without requiring a specific or immediate time constraint for the action to occur. The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more. Combinations such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, where any such combinations may contain one or more member or members of A, B, or C. Sets should be interpreted as a set of elements where the elements number one or more. Accordingly, for a set of X, X would include one or more elements. All structural and functional equivalents to the elements of the various aspects described throughout this disclosure that are known or later come to be known to those of ordinary skill in the art are expressly incorporated herein by reference and are encompassed by the claims. Moreover, nothing disclosed herein is dedicated to the public regardless of whether such disclosure is explicitly recited in the claims. The words “module,” “mechanism,” “element,” “device,” and the like may not be a substitute for the word “means.” As such, no claim element is to be construed as a means plus function unless the element is expressly recited using the phrase “means for.”

As used herein, the phrase “based on” shall not be construed as a reference to a closed set of information, one or more conditions, one or more factors, or the like. In other words, the phrase “based on A” (where “A” may be information, a condition, a factor, or the like) shall be construed as “based at least on A” unless specifically recited differently.

The following example clauses are illustrative only and may be combined with other aspects or teachings described herein, without limitation.
Clause 1. A medical device, comprising: a pusher; and a clot-engaging device coupled to the pusher, the clot-engaging device including a plurality of bristles from a surface of the clot-engaging device.
Clause 2. The medical device of clause 1, wherein each bristle of the plurality of bristles comprises a first end attached to the outer surface of the clot-engaging device.
Clause 3. The medical device of clause 2, wherein each bristle of the plurality of bristles further comprises a second end attached to the outer surface of the clot-engaging device, each bristle of the plurality of bristles forming a loop with the surface.
Clause 4. The medical device of clause 1, wherein the plurality of bristles comprises a first set of bristles and a second set of bristles, the second set of bristles being different than the first set of bristles.
Clause 5. The medical device of clause 4, wherein the first set of bristles and the second set of bristles differ by at least one of a body shape, a tip shape, a length, a width, or a material composition.
Clause 6. The medical device of clause 5, wherein the tip shape of each bristle in the first set of bristles is one of rounded, circular, square, rectangular, polygonal, triangular, helical, oval, or trapezoidal, and wherein the tip shape of each bristle in the second set of bristles is different from the first set of bristles.
Clause 7. The medical device of clause 1, wherein each bristle of the plurality of bristles further comprises a tip shape that is one of rounded, circular, square, rectangular, polygonal, triangular, helical, oval, or trapezoidal.
Clause 8. The medical device of clause 5, wherein the body shape of each bristle in the first set of bristles is one of straight, zigzagged, or bent, and wherein the body shape of each bristle in the second set of bristles is different from the first set of bristles.
Clause 9. The medical device of clause 5, wherein the material composition of each bristle in the first set of bristles is one of polymer, polymer-based, or nickel titanium.
Clause 10. The medical device of clause 9, wherein the material composition is polymer.
Clause 11. The medical device of clause 9, wherein the material composition is based on the polymer having a Shore hardness less than a particular value.
Clause 12. The medical device of clause 9, wherein the material composition is based on the polymer having a durometer less than a particular value.
Clause 13. The medical device of clause 4, wherein the first set of bristles is attached to a first cross-sectional portion of the outer surface of the clot-engaging device, and the second set of bristles is attached to a second cross-sectional portion of the outer surface of the clot-engaging device, the first cross-sectional portion and the second cross-sectional portion being separated by a bristle-free region on the outer surface of the clot-engaging device.
Clause 14. The medical device of clause 13, wherein the first set of bristles or the second set of bristles comprises hook-shaped bristles, each of the hook-shaped bristles having a curved distal bristle tip, the curved distal bristle tip pointing to one of a distal end of the clot-engaging device or a non-distal end of the clot-engaging device.
Clause 15. The medical device of clause 14, wherein at least one of the first set of bristles or the second set of bristles comprises only the hook-shaped bristles.
Clause 16. The medical device of clause 14, wherein the curved distal bristle tip of each of the hook-shaped bristles has a curvature of at least 90 degrees, 135 degrees, or 180 degrees.
Clause 17. The medical device of clause 14, wherein the curved distal bristle tip of each of the hook-shaped bristles has a helical shape.
Clause 18. The medical device of clause 14, wherein at least a portion of a body of each bristle of the plurality of bristles extends linearly in a radial direction from a center of the clot-engaging device.
Clause 19. The medical device of clause 18, wherein the portion of the body of each bristle of the plurality of bristles is coupled to the outer surface.
Clause 20. The medical device of clause 1, wherein at least one bristle of the plurality of bristles is pointing in a tangential direction away from the outer surface of the clot-engaging device.
Clause 21. The medical device of clause 1, wherein the clot-engaging device is conically shaped.
Clause 22. The medical device of clause 1, further comprising: a catheter coupled to at least one of the clot-engaging device or the pusher, wherein, in a delivery configuration, the clot-engaging device is inside of the catheter, and wherein, in a deployed configuration, the clot-engaging device is outside of the catheter.
Clause 23. The medical device of clause 22, wherein, in the deployed configuration, at least one of the plurality of bristles is configured to engage a clot.
Clause 24. The medical device of clause 22, wherein, in the delivery configuration, at least one of the plurality of bristles is in a resting configuration, in the deployed configuration, at least one of the plurality of bristles is in a clot-engaging configuration different from the resting configuration.
Clause 25. The medical device of clause 1, wherein the plurality of bristles extends from the outer surface of the clot-engaging device at a radius from an axial center of the clot-engaging device, the clot-engaging device further comprises an inflatable balloon coupled to the pusher, and the inflatable balloon is configured to change the radius of the plurality of bristles from the axial center of the clot-engaging device.
Clause 26. The medical device of clause 25, wherein the clot-engaging device further comprises an intermediate layer exterior to the inflatable balloon, the plurality of bristles is coupled to the intermediate layer, and the inflatable balloon is configured to change the radius of the intermediate layer from the axial center of the clot-engaging device.
Clause 27. The medical device of clause 26, wherein the intermediate layer comprises a flexible material that remains coupled to the inflatable balloon when the inflatable balloon expands from a deflated configuration to an inflated configuration, wherein the radius is greater in the inflated configuration than in the deflated configuration.
Clause 28. The medical device of clause 25, further comprising a spring shaft located, at least in part, along an axial length of the inflatable balloon.
Clause 29. The medical device of clause 28, wherein the spring shaft comprises at least one coil.
Clause 30. The medical device of clause 28, wherein the spring shaft is coupled to the inflatable balloon at two or more axial locations along the inflatable balloon.
Clause 31. The medical device of clause 1, wherein the clot-engaging device further comprises at least one coil located in a coil region of the clot-engaging device between a first bristle region and a second bristle region.
Clause 32. The medical device of clause 31, wherein the at least one coil is a non-metal material.
Clause 33. The medical device of clause 31, wherein the at least one coil is a polymer based material.
Clause 34. The medical device of clause 31, wherein the at least one coil comprises a metal coil.
Clause 35. The medical device of clause 31, wherein each of the at least one coil is located in one of a plurality of coil regions on the surface, wherein each of the plurality of coil regions is wrapped around a circumference of the clot-engaging device, wherein each of the at least one coil is separated by a set of bristles of the plurality of bristles.
Clause 36. The medical device of clause 31, wherein the clot-engaging device or the pusher includes at least one radio-opaque marker.
Clause 37. A medical device, comprising: a pusher; and a clot-engaging device coupled to the pusher, the clot-engaging device including at least one semi-continuous protrusion or groove or at least one cavity on an outer surface or an inner surface.
Clause 38. The medical device of clause 37, wherein each cavity of the at least one cavity has a cavity depth that is less than a thickness of the clot-engaging device.
Clause 39. The medical device of clause 38, wherein the at least one cavity overlaps with the at least one semi-continuous protrusion or groove.
Clause 40. The medical device of clause 38, wherein the at least one cavity does not overlap with the at least one semi-continuous protrusion or groove.
Clause 41. The medical device of clause 37, wherein the at least one cavity is located at the outer surface.
Clause 42. The medical device of clause 37, wherein the at least one cavity is located at the inner surface.
Clause 43. The medical device of clause 37, wherein the at least one semi-continuous protrusion or groove is located at the outer surface.
Clause 44. The medical device of clause 37, wherein the at least one semi-continuous protrusion or groove is located at the inner surface.
Clause 45. The medical device of clause 37, wherein the at least one semi-continuous protrusion or groove forms a plurality of kite-shaped regions on the outer surface of the clot-engaging device, and wherein each cavity of the at least one cavity is located within the plurality of kite-shaped regions.
Clause 46. The medical device of clause 45, wherein the at least one cavity is substantially centered within the plurality of kite-shaped regions.
Clause 47. The medical device of clause 37, wherein the at least one semi-continuous protrusion or groove is based on a sweep cutout.
Clause 48. The medical device of clause 37, wherein the at least one semi-continuous protrusion or groove has a helical pattern.
Clause 49. The medical device of clause 37, wherein the at least one semi-continuous protrusion or groove comprises a plurality of protrusions or grooves along the outer surface.
Clause 50. The medical device of clause 49, wherein the plurality of protrusions or grooves each has a helical pattern.
Clause 51. The medical device of clause 49, wherein the plurality of protrusions or grooves comprises one or more pairs of protrusions or grooves, wherein each of the one or more pairs of protrusions or grooves comprise a first semi-continuous protrusion or groove having a clockwise helical pattern and a second semi-continuous protrusion or groove having a counterclockwise helical pattern.
Clause 52. The medical device of clause 51, wherein the one or more pairs of protrusions or grooves comprise at least two pairs of protrusions or grooves.
Clause 53. The medical device of clause 51, wherein the one or more pairs of protrusions or grooves comprise at least three pairs of protrusions or grooves.
Clause 54. The medical device of clause 51, wherein the protrusions having the clockwise helical pattern are spaced substantially equidistant from one another on the outer surface of the clot-engaging device, or wherein the protrusions having the counterclockwise helical pattern are spaced substantially equidistant from one another on the outer surface of the clot-engaging device.
Clause 55. The medical device of clause 49, wherein the plurality of protrusions or grooves comprises at least a first semi-continuous groove having a first depth and a second semi-continuous groove having a second depth, wherein the first depth and the second depth are both less than a thickness of the clot-engaging device, and wherein the first depth and the second depth are unequal to each other.
Clause 56. The medical device of clause 37, wherein a depth of the at least one semi-continuous protrusion or groove is a variable depth.
Clause 57. The medical device of clause 37, wherein the clot-engaging device or the pusher includes at least one radio-opaque marker.
Clause 58. The medical device of clause 37, wherein the clot-engaging device is conically shaped.
Clause 59. The medical device of clause 37, further comprising an inflatable balloon coupled to the pusher, wherein the at least one semi-continuous protrusion or groove or the at least one cavity is located at the outer surface, and wherein the clot-engaging device is exterior to the inflatable balloon.
Clause 60. The medical device of clause 59, wherein the at least one semi-continuous protrusion or groove or the at least one cavity is coupled to the inflatable balloon by an intermediate layer, wherein the at least one semi-continuous protrusion or groove or the at least one cavity is coupled to an exterior surface of the intermediate layer, and the inflatable balloon is coupled to an interior surface of the intermediate layer.
Clause 61. The medical device of clause 60, wherein the intermediate layer comprises a flexible material that remains coupled to the inflatable balloon when the inflatable balloon expands from a deflated configuration to an inflated configuration, wherein the radius is greater in the inflated configuration than in the deflated configuration.
Clause 62. The medical device of clause 60, wherein the clot-engaging device further comprises a spring shaft located, at least in part, along an axial length of the inflatable balloon.
Clause 63. The medical device of clause 62, wherein the spring shaft comprises at least one coil.
Clause 64. The medical device of clause 62, wherein the spring shaft is coupled to the inflatable balloon at two or more axial locations along the balloon catheter.
Clause 65. The medical device of clause 37, further comprising: a catheter coupled to at least one of the clot-engaging device or the pusher, wherein, in a delivery configuration, the clot-engaging device is inside of the catheter, and wherein, in a deployed configuration, the clot-engaging device is outside of the catheter.
Clause 66. A medical device, comprising: a pusher with a distal end having an elongated member; a plurality of bristles, wherein each bristle has a proximal bristle end distributed along the elongated member and a distal bristle tip not distributed along the elongated member, and wherein the plurality of bristles comprises: a first set of bristles distributed along a first cross-sectional portion of the elongated member, and a second set of bristles distributed along a second cross-sectional portion of the elongated member; and wherein the elongated member has a first bristle-free region located between the first cross-sectional portion and the second cross-sectional portion of the elongated member.
Clause 67. The medical device of clause 66, wherein, in a delivery configuration, the medical device is within a lumen of a catheter, and wherein, in a deployed configuration, the medical device is distal to the catheter.
Clause 68. The medical device of clause 66, wherein each bristle of the plurality of bristles has a rounded tip and the elongated member is polymer-based.
Clause 69. The medical device of clause 66, wherein the first set of bristles is parallel to the second set of bristles.
Clause 70. The medical device of clause 66, wherein each bristle of the plurality of bristles has a straight bristle portion that is aligned in a radial direction extending outwardly from a center of the elongated member.
Clause 71. The medical device of clause 70, wherein the straight bristle portion of each bristle of the plurality of bristles is located at least in part at the proximal bristle end of the bristle.
Clause 72. The medical device of clause 66, wherein at least one of the first set of bristles or the second set of bristles comprise hook-shaped bristles, wherein each of the hook-shaped bristles has a curved distal bristle tip, the curved distal bristle tip pointing to the distal end or a non-distal end.
Clause 73. The medical device of clause 72, wherein at least one of the first set of bristles or the second set of bristles comprises only the hook-shaped bristles, wherein each of the hook-shaped bristles has the curved distal bristle tip.
Clause 74. The medical device of clause 73, wherein the curved distal bristle tip of each of the hook-shaped bristles has a curvature of at least 90 degrees.
Clause 75. The medical device of clause 73, wherein the curved distal bristle tip of each of the hook-shaped bristles has a curvature of at least 135 degrees.
Clause 76. The medical device of clause 73, wherein the curved distal bristle tip of each of the hook-shaped bristles has a curvature of at least 180 degrees.
Clause 77. The medical device of clause 73, wherein the curved distal bristle tip of each of the hook-shaped bristles has a helical shape.
Clause 78. The medical device of clause 66, wherein each bristle of the plurality of bristles is a hook-shaped bristles wherein each of the hook-shaped bristles has a curved distal bristle tip, the curved distal bristle tip pointing to the distal end or a non-distal end.
Clause 79. The medical device of clause 66, wherein at least one of the first set of bristles or the second set of bristles comprises bristles of a circular shape, a square shape, a rectangular shape, a polygon shape, a triangular shape, an oval shape, a trapezoid shape, a straight line longitudinally, a zigzag line longitudinally, or a bent line longitudinally.
Clause 80. The medical device of clause 66, wherein the first cross-sectional portion and the second cross-sectional portion are located at a tapered region of the elongated member, wherein the first cross-sectional portion of the elongated member has a first diameter, wherein the second cross-sectional portion of the elongated member has a second diameter, and wherein the first diameter is larger than the second diameter.
Clause 81. The medical device of clause 80, wherein each bristle in the first set of bristles has a first length, wherein each bristle in the second set of bristles has a second length, and wherein the first length equals the second length.
Clause 82. The medical device of clause 80, wherein each bristle in the first set of bristles has a first length, wherein each bristle in the second set of bristles has a second length, and wherein the first length is greater than the second length.
Clause 83. The medical device of clause 80, wherein each bristle in the first set of bristles has a first length, wherein each bristle in the second set of bristles has a second length, and wherein the first length is less than the second length.
Clause 84. The medical device of clause 80, wherein a first subset of bristles in the first set of bristles and a second subset of bristles in the second set of bristles has a first length, wherein a third subset of bristles in the first set of bristles and a fourth subset of bristles in the second set of bristles has a second length, and wherein the first length is less than the second length.
Clause 85. The medical device of clause 66, wherein at least one bristle of the plurality of bristles is pointing in a tangential direction away from a circle associated with the elongated member.
Clause 86. The medical device of clause 66, wherein the first set of bristles are distributed along a circumference of the elongated member based on a pattern.
Clause 87. The medical device of clause 86, wherein the pattern is based on the first set of bristles being associated with a same axial distance associated with the elongated member.
Clause 88. The medical device of clause 86, wherein the pattern is based on the first set of bristles being associated with a particular range of axial distance of the elongated member.
Clause 89. The medical device of clause 66, wherein the elongated member is hollow.
Clause 90. The medical device of clause 89, wherein the plurality of bristles is distributed along an inner surface of the elongated member.
Clause 91. The medical device of clause 66, wherein the elongated member is a hollow tube.
Clause 92. The medical device of clause 66, wherein the elongated member is non-hollow.
Clause 93. The medical device of clause 66, wherein the elongated member is cylindrical.
Clause 94. The medical device of clause 66, wherein the plurality of bristles is distributed along an outer surface of the elongated member.
Clause 95. The medical device of clause 66, wherein the first set of bristles is located at a first bristle region of the elongated member and the second set of bristles located at a second bristle region of the elongated member, and wherein the first bristle region and the second bristle region are spaced apart by a distance along the elongated member.
Clause 96. The medical device of clause 95, wherein the first bristle region is located at the first cross-sectional portion of the elongated member, wherein the second bristle region is located at the second cross-sectional portion of the elongated member, and wherein the first cross-sectional portion and the second cross-sectional portion are spaced apart on the elongated member by a longitudinal distance.
Clause 97. The medical device of clause 95, wherein the first bristle region is located at a first cross-section of the elongated member, wherein the first bristle region is located at the first cross-sectional portion of the elongated member, wherein the second bristle region is located at the second cross-sectional portion of the elongated member, and wherein the first cross-sectional portion and the second cross-sectional portion are spaced apart at different circumferential regions of the elongated member.
Clause 98. The medical device of clause 95, wherein the elongated member further comprises at least one coil located in a coil region of the elongated member between the first bristle region and the second bristle region.
Clause 99. The medical device of clause 98, wherein the at least one coil is a non-metal.
Clause 100. The medical device of clause 98, wherein the at least one coil is a polymer.
Clause 101. The medical device of clause 98, wherein the at least one coil is a metal.
Clause 102. The medical device of clause 95, further comprising a plurality of coils, wherein each of the plurality of coils is located in one of a plurality of coil regions on an outer surface of the elongated member, wherein each of the plurality of coil regions is wrapped around a circumference of the elongated member, wherein each of the plurality of coil regions is proximal to at least one of the plurality of bristles, and wherein each of the plurality of coil regions is distal to at least another one of the plurality of bristles.
Clause 103. The medical device of clause 95, wherein the first set of bristles comprise a first plurality of bristles having a substantially uniform first length.
Clause 104. The medical device of clause 103, wherein the second set of bristles comprise a second plurality of bristles having a substantially uniform second length.
Clause 105. The medical device of clause 104, wherein the substantially uniform first length is less than the substantially uniform second length.
Clause 106. The medical device of clause 66, wherein the elongated member further comprises an inflatable balloon.
Clause 107. The medical device of clause 106, wherein the elongated member further comprises an intermediate layer, wherein the plurality of bristles is coupled to the intermediate layer, and the inflatable balloon is configured to change the radius of the intermediate layer from an axial center of the elongated member.
Clause 108. The medical device of clause 107, wherein the intermediate layer comprises a flexible material that remains coupled to the inflatable balloon when the inflatable balloon expands from a deflated configuration to an inflated configuration, wherein the radius is greater in the inflated configuration than in the deflated configuration.
Clause 109. The medical device of clause 106, wherein the elongated member further comprises a spring shaft located, at least in part, along an axial length of the inflatable balloon.
Clause 110. The medical device of clause 109, wherein the spring shaft comprises at least one coil.
Clause 111. The medical device of clause 109, wherein the spring shaft is coupled to the inflatable balloon at two or more axial locations along the balloon catheter.
Clause 112. The medical device of clause 66, wherein the elongated member has at least one semi-continuous protrusion or groove or at least one cavity along an outer surface or an inner surface of the elongated member with a depth that is less than a thickness of the elongated member.
Clause 113. The medical device of clause 66, wherein the elongated member has at least one semi-continuous protrusion or groove or along an outer surface or an inner surface of the elongated member with a depth that is less than a thickness of the elongated member at least one cavity along the outer surface or the inner surface of the elongated member.
Clause 114. The medical device of clause 113, wherein each cavity of the at least one cavity has a cavity depth that is less than the thickness of the elongated member.
Clause 115. The medical device of clause 114, wherein the at least one cavity overlaps with the at least one semi-continuous protrusion or groove.
Clause 116. The medical device of clause 114, wherein the at least one cavity does not overlap with the at least one semi-continuous protrusion or groove.
Clause 117. The medical device of clause 113, wherein the at least one cavity is located at the outer surface.
Clause 118. The medical device of clause 113, wherein the at least one cavity is located at the inner surface.
Clause 119. The medical device of clause 113, wherein the at least one semi-continuous protrusion or groove is located at the outer surface.
Clause 120. The medical device of clause 113, wherein the at least one semi-continuous protrusion or groove is located at the inner surface.
Clause 121. The medical device of clause 113, wherein the at least one semi-continuous protrusion or groove forms a plurality of kite-shaped regions on the outer surface of the elongated member, and wherein each cavity of the at least one cavity is located within the plurality of kite-shaped regions.
Clause 122. The medical device of clause 121, wherein the at least one cavity is substantially centered within the plurality of kite-shaped regions.
Clause 123. The medical device of clause 113, wherein the at least one semi-continuous protrusion or groove is based on a sweep cutout.
Clause 124. The medical device of clause 113, wherein the at least one semi-continuous protrusion or groove has a helical pattern.
Clause 125. The medical device of clause 113, wherein the at least one semi-continuous protrusion or groove comprises a plurality of protrusions or grooves along the outer surface or the inner surface.
Clause 126. The medical device of clause 125, wherein the plurality of protrusions or grooves each has a helical pattern.
Clause 127. The medical device of clause 125, wherein the plurality of protrusions or grooves comprises one or more pairs of protrusions or grooves, wherein each of the one or more pairs of protrusions or grooves comprise a first semi-continuous protrusion or groove having a clockwise helical pattern and a second semi-continuous protrusion or groove having a counterclockwise helical pattern.
Clause 128. The medical device of clause 127, wherein the one or more pairs of protrusions or grooves comprise at least two pairs of protrusions or grooves.
Clause 129. The medical device of clause 127, wherein the one or more pairs of protrusions or grooves comprise at least three pairs of protrusions or grooves.
Clause 130. The medical device of clause 127, wherein the protrusions having the clockwise helical pattern are spaced substantially equidistant from one another on the outer surface of the elongated member, or wherein the protrusions having the counterclockwise helical pattern are spaced substantially equidistant from one another on the outer surface of the elongated member.
Clause 131. The medical device of clause 125, wherein the plurality of protrusions or grooves comprises at least a first semi-continuous groove having a first depth and a second semi-continuous groove having a second depth, wherein the first depth and the second depth are both less than the thickness of the elongated member, and wherein the first depth and the second depth are unequal to each other.
Clause 132. The medical device of clause 113, wherein the depth of the at least one semi-continuous protrusion or groove is a variable depth.
Clause 133. The medical device of clause 113, wherein the elongated member or the pusher includes at least one radio-opaque marker.
Clause 134. The medical device of clause 113, wherein the elongated member is conically shaped.
Clause 135. The medical device of clause 113, wherein the at least one semi-continuous protrusion or groove or the at least one cavity is located at the outer surface, and wherein the elongated member further comprises an inflatable balloon.
Clause 136. A medical device, comprising: a pusher with a distal end having an elongated member; a plurality of bristles, wherein each bristle has a proximal bristle end distributed along the elongated member and a distal bristle tip not distributed along the elongated member, and wherein the plurality of bristles comprises: a first set of bristles distributed along a first longitudinal portion of the elongated member, and a second set of bristles distributed along a second longitudinal portion of the elongated member; and wherein the elongated member has a first bristle-free region located between the first longitudinal portion and the second longitudinal portion of the elongated member.
Clause 137. The medical device of clause 136, wherein, in a delivery configuration, the medical device is within a lumen of a catheter, and wherein, in a deployed configuration, the medical device is distal to the catheter.
Clause 138. The medical device of clause 136, wherein each bristle of the plurality of bristles has a rounded tip and the elongated member is polymer-based.
Clause 139. The medical device of clause 136, wherein the first set of bristles is parallel to the second set of bristles.
Clause 140. The medical device of clause 136, wherein each bristle of the plurality of bristles has a straight bristle portion that is aligned in a radial direction extending outwardly from a center of the elongated member.
Clause 141. The medical device of clause 140, wherein the straight bristle portion of each bristle of the plurality of bristles is located at least in part at the proximal bristle end of the bristle.
Clause 142. The medical device of clause 136, wherein at least one of the first set of bristles or the second set of bristles comprise hook-shaped bristles, wherein each of the hook-shaped bristles has a curved distal bristle tip, the curved distal bristle tip pointing to the distal end or a non-distal end.
Clause 143. The medical device of clause 142, wherein at least one of the first set of bristles or the second set of bristles comprises only the hook-shaped bristles, wherein each of the hook-shaped bristles has the curved distal bristle tip.
Clause 144. The medical device of clause 143, wherein the curved distal bristle tip of each of the hook-shaped bristles has a curvature of at least 90 degrees.
Clause 145. The medical device of clause 143, wherein the curved distal bristle tip of each of the hook-shaped bristles has a curvature of at least 135 degrees.
Clause 146. The medical device of clause 143, wherein the curved distal bristle tip of each of the hook-shaped bristles has a curvature of at least 180 degrees.
Clause 147. The medical device of clause 143, wherein the curved distal bristle tip of each of the hook-shaped bristles has a helical shape.
Clause 148. The medical device of clause 136, wherein each bristle of the plurality of bristles is a hook-shaped bristle that has a curved distal bristle tip, the curved distal bristle tip pointing to the distal end or a non-distal end.
Clause 149. The medical device of clause 136, wherein at least one of the first set of bristles or the second set of bristles comprises bristles of a circular shape, a square shape, a rectangular shape, a polygon shape, a triangular shape, an oval shape, a trapezoid shape, a straight line longitudinally, a zigzag line longitudinally, or a bent line longitudinally.
Clause 150. The medical device of clause 136, wherein the first longitudinal portion and the second longitudinal portion are located at a tapered region of the elongated member.
Clause 151. The medical device of clause 150, wherein each bristle in the first set of bristles has a first length, wherein each bristle in the second set of bristles has a second length, and wherein the first length equals the second length.
Clause 152. The medical device of clause 150, wherein each bristle in the first set of bristles has a first length, wherein each bristle in the second set of bristles has a second length, and wherein the first length is greater than the second length.
Clause 153. The medical device of clause 150, wherein each bristle in the first set of bristles has a first length, wherein each bristle in the second set of bristles has a second length, and wherein the first length is less than the second length.
Clause 154. The medical device of clause 150, wherein a first subset of bristles in the first set of bristles and a second subset of bristles in the second set of bristles has a first length, wherein a third subset of bristles in the first set of bristles and a fourth subset of bristles in the second set of bristles has a second length, and wherein the first length is less than the second length.
Clause 155. The medical device of clause 146, wherein at least one bristle of the plurality of bristles is pointing in a tangential direction away from a circle associated with the elongated member.
Clause 156. The medical device of clause 146, wherein the first set of bristles are distributed along a circumference of the elongated member.
Clause 157. The medical device of clause 156, wherein the first set of bristles are distributed along the circumference of the elongated member based on a pattern.
Clause 158. The medical device of clause 157, wherein the pattern is based on the first set of bristles being associated with a same axial distance associated with the elongated member.
Clause 159. The medical device of clause 157, wherein the pattern is based on the first set of bristles being associated with a particular range of axial distance of the elongated member.
Clause 160. The medical device of clause 136, wherein the elongated member is hollow.
Clause 161. The medical device of clause 160, wherein the plurality of bristles is distributed along an inner surface of the elongated member.
Clause 162. The medical device of clause 136, wherein the elongated member is a hollow tube.
Clause 163. The medical device of clause 136, wherein the elongated member is non-hollow.
Clause 164. The medical device of clause 136, wherein the elongated member is cylindrical.
Clause 165. The medical device of clause 136, wherein the plurality of bristles is distributed along an outer surface of the elongated member.
Clause 166. The medical device of clause 136, wherein the first set of bristles is located at a first bristle region of the elongated member and the second set of bristles located at a second bristle region of the elongated member, and wherein the first bristle region and the second bristle region are spaced apart by a distance along the elongated member.
Clause 167. The medical device of clause 166, wherein the first bristle region is located at the first longitudinal portion of the elongated member, wherein the second bristle region is located at the second longitudinal portion of the elongated member, and wherein the first longitudinal portion and the second longitudinal portion are spaced apart on the elongated member by a distance.
Clause 168. The medical device of clause 166, wherein the elongated member further comprises at least one coil located in a coil region of the elongated member between the first bristle region and the second bristle region.
Clause 169. The medical device of clause 168, wherein the at least one coil is a non-metal.
Clause 170. The medical device of clause 168, wherein the at least one coil is a polymer.
Clause 171. The medical device of clause 168, wherein the at least one coil is a metal.
Clause 172. The medical device of clause 166, further comprising a plurality of coils, wherein each of the plurality of coils is located in one of a plurality of coil regions on an outer surface of the elongated member, wherein each of the plurality of coil regions is wrapped around a circumference of the elongated member, wherein each of the plurality of coil regions is proximal to at least one of the plurality of bristles, and wherein each of the plurality of coil regions is distal to at least another one of the plurality of bristles.
Clause 173. The medical device of clause 166, wherein the first set of bristles comprise a first plurality of bristles having a substantially uniform first length.
Clause 174. The medical device of clause 173, wherein the second set of bristles comprise a second plurality of bristles having a substantially uniform second length.
Clause 175. The medical device of clause 174, wherein the substantially uniform first length is less than the substantially uniform second length.
Clause 176. The medical device of clause 136, wherein the elongated member further comprises an inflatable balloon.
Clause 177. The medical device of clause 176, wherein the elongated member further comprises an intermediate layer, wherein the plurality of bristles is coupled to the intermediate layer, and the inflatable balloon is configured to change a radius of the intermediate layer from an axial center of the elongated member.
Clause 178. The medical device of clause 177, wherein the intermediate layer comprises a flexible material that remains coupled to the inflatable balloon when the inflatable balloon expands from a deflated configuration to an inflated configuration, wherein the radius is greater in the inflated configuration than in the deflated configuration.
Clause 179. The medical device of clause 176, wherein the elongated member further comprises a spring shaft located, at least in part, along an axial length of the inflatable balloon.
Clause 180. The medical device of clause 179, wherein the spring shaft comprises at least one coil.
Clause 181. The medical device of clause 179, wherein the spring shaft is coupled to the inflatable balloon at two or more axial locations along the balloon catheter.
Clause 182. The medical device of clause 136, wherein the elongated member has at least one semi-continuous protrusion or groove or at least one cavity along an outer surface or an inner surface of the elongated member with a depth that is less than a thickness of the elongated member.
Clause 183. The medical device of clause 136, wherein the elongated member has at least one semi-continuous protrusion or groove or at least one cavity along an outer surface or an inner surface of the elongated member with a depth that is less than a thickness of the elongated member.
Clause 184. The medical device of clause 183, wherein each cavity of the at least one cavity has a cavity depth that is less than the thickness of the elongated member.
Clause 185. The medical device of clause 184, wherein the at least one cavity overlaps with the at least one semi-continuous protrusion or groove.
Clause 186. The medical device of clause 184, wherein the at least one cavity does not overlap with the at least one semi-continuous protrusion or groove.
Clause 187. The medical device of clause 183, wherein the at least one cavity is located at the outer surface.
Clause 188. The medical device of clause 183, wherein the at least one cavity is located at the inner surface.
Clause 189. The medical device of clause 183, wherein the at least one semi-continuous protrusion or groove is located at the outer surface.
Clause 190. The medical device of clause 183, wherein the at least one semi-continuous protrusion or groove is located at the inner surface.
Clause 191. The medical device of clause 183, wherein the at least one semi-continuous protrusion or groove forms a plurality of kite-shaped regions on the outer surface of the elongated member, and wherein each cavity of the at least one cavity is located within the plurality of kite-shaped regions.
Clause 192. The medical device of clause 191, wherein the at least one cavity is substantially centered within the plurality of kite-shaped regions.
Clause 193. The medical device of clause 183, wherein the at least one semi-continuous protrusion or groove is based on a sweep cutout.
Clause 194. The medical device of clause 183, wherein the at least one semi-continuous protrusion or groove has a helical pattern.
Clause 195. The medical device of clause 183, wherein the at least one semi-continuous protrusion or groove comprises a plurality of protrusions or grooves along the outer surface or the inner surface.
Clause 196. The medical device of clause 195, wherein the plurality of protrusions or grooves each has a helical pattern.
Clause 197. The medical device of clause 195, wherein the plurality of protrusions or grooves comprises one or more pairs of protrusions or grooves, wherein each of the one or more pairs of protrusions or grooves comprise a first semi-continuous protrusion or groove having a clockwise helical pattern and a second semi-continuous protrusion or groove having a counterclockwise helical pattern.
Clause 198. The medical device of clause 197, wherein the one or more pairs of protrusions or grooves comprise at least two pairs of protrusions or grooves.
Clause 199. The medical device of clause 197, wherein the one or more pairs of protrusions or grooves comprise at least three pairs of protrusions or grooves.
Clause 200. The medical device of clause 197, wherein the protrusions having the clockwise helical pattern are spaced substantially equidistant from one another on the outer surface of the elongated member, or wherein the protrusions having the counterclockwise helical pattern are spaced substantially equidistant from one another on the outer surface of the elongated member.
Clause 201. The medical device of clause 195, wherein the plurality of protrusions or grooves comprises at least a first semi-continuous groove having a first depth and a second semi-continuous groove having a second depth, wherein the first depth and the second depth are both less than the thickness of the elongated member, and wherein the first depth and the second depth are unequal to each other.
Clause 202. The medical device of clause 183, wherein the depth of the at least one semi-continuous protrusion or groove is a variable depth.
Clause 203. The medical device of clause 183, wherein the elongated member or the pusher includes at least one radio-opaque marker.
Clause 204. The medical device of clause 183, wherein the elongated member is conically shaped.
Clause 205. The medical device of clause 183, wherein the at least one semi-continuous protrusion or groove or the at least one cavity is located at the outer surface, and wherein the elongated member further comprises an inflatable balloon.
Clause 206. A medical device, comprising: an elongated member having an outer surface, inner lumen, and a tube body therebetween, wherein the elongated member has at least one semi-continuous protrusion or groove along the outer surface or an inner surface of the elongated member with a depth that is less than a thickness of the elongated member.
Clause 207. The medical device of clause 206, wherein the at least one semi-continuous protrusion or groove is based on a sweep cutout.
Clause 208. The medical device of clause 206, wherein the at least one semi-continuous protrusion or groove has a helical pattern.
Clause 209. The medical device of clause 206, wherein the at least one semi-continuous protrusion or groove comprises a plurality of protrusions or grooves along the outer surface.
Clause 210. The medical device of clause 209, wherein the plurality of protrusions or grooves each has a helical pattern.
Clause 211. The medical device of clause 209, wherein the plurality of protrusions or grooves comprises one or more pairs of protrusions or grooves, wherein each of the one or more pairs of protrusions or grooves comprise a first semi-continuous protrusion or groove having a clockwise helical pattern and a second semi-continuous protrusion or groove having a counterclockwise helical pattern.
Clause 212. The medical device of clause 211, wherein the one or more pairs of protrusions or grooves comprise at least two pairs of protrusions or grooves.
Clause 213. The medical device of clause 211, wherein at least one semi-continuous protrusion or groove having the clockwise helical pattern is spaced substantially equidistant from one another on the outer surface of the elongated member, or wherein at least one other semi-continuous protrusion or groove having the counterclockwise helical pattern is spaced substantially equidistant from one another on the outer surface of the elongated member.
Clause 214. The medical device of clause 209, wherein the plurality of protrusions or grooves comprises at least a first semi-continuous groove having a first depth and a second semi-continuous groove having a second depth, wherein the first depth and the second depth are both less than the thickness of the elongated member, and wherein the first depth and the second depth are unequal to each other.
Clause 215. The medical device of clause 206, wherein the depth of the at least one semi-continuous protrusion or groove is a variable depth.
Clause 216. The medical device of clause 206, further comprising a plurality of cavities on the outer surface of the elongated member, wherein each of the plurality of cavities has a cavity depth that is less than the thickness of the elongated member.
Clause 217. The medical device of clause 216, wherein at least one of the plurality of cavities overlaps with the at least one semi-continuous protrusion or groove.
Clause 218. The medical device of clause 216, wherein the plurality of cavities does not overlap with the at least one semi-continuous protrusion or groove on the outer surface of the elongated member.
Clause 219. The medical device of clause 216, wherein the plurality of cavities is spaced apart from the at least one semi-continuous protrusion or groove on the outer surface of the elongated member.
Clause 220. The medical device of clause 216, wherein the plurality of cavities is an array of cavities spaced apart from one another.
Clause 221. The medical device of clause 216, wherein the at least one semi-continuous protrusion or groove forms a plurality of kite-shaped regions on the outer surface of the elongated member, and wherein each of the plurality of cavities are located within the plurality of kite-shaped regions.
Clause 222. The medical device of clause 221, wherein the plurality of cavities is substantially centered within the plurality of kite-shaped regions.
Clause 223. A medical device, comprising: an elongated member having an outer surface, inner lumen, and a tube body therebetween, wherein the elongated member has a plurality of cavities along the outer surface each having a depth that is less than a thickness of the elongated member.
Clause 224. The medical device of clause 223, wherein the plurality of cavities is an array of cavities spaced apart from one another.
Clause 225. The medical device of clause 223, wherein the plurality of cavities comprises a first cavity having a first volume, a second cavity having a second volume, and wherein the first volume and second volume are different.
Clause 226. The medical device of clause 225, wherein the first cavity has a first depth, the second cavity has a second depth, and wherein the first depth and second depth are different.
Clause 227. The medical device of clause 225, wherein the first cavity has a first diameter at the outer surface of the elongated member, the second cavity has a second diameter at the outer surface of the elongated member, and wherein the first diameter and the second diameter are different.
Clause 228. The medical device of clause 225, where in a collection configuration a first foreign body is at least partially within the first cavity and a second foreign body is at least partially within the second cavity.
Clause 229. The medical device of clause 228, wherein the first foreign body and the second foreign body are blood clots of different sizes.
Clause 230. The medical device of clause 228, further comprising a catheter having an inner catheter lumen, where in the collection configuration the first foreign body and the second foreign body are trapped between the plurality of cavities and the inner catheter lumen.
Clause 231. The medical device of clause 223, wherein the plurality of cavities comprise at least four cavities spaced apart equally along the outer surface of the elongated member.
Clause 232. The medical device of clause 223, where in a collection configuration a first foreign body is at least partially within at least one semi-continuous protrusion or groove.
Clause 233. The medical device of clause 232, wherein the first foreign body is a blood clot.
Clause 234. The medical device of clause 232, further comprising a catheter having an inner catheter lumen, where in the collection configuration the first foreign body is trapped between the at least one semi-continuous protrusion or groove and the inner catheter lumen.
Clause 235. A medical device, comprising: an elongated member having an outer surface, inner lumen, and a tube body therebetween; and at least one helical protrusion or groove that spirals helically and longitudinally along the outer surface of the elongated member for one or more rotations.
Clause 236. The medical device of clause 235, wherein the at least one helical protrusion or groove comprises a first helical protrusion or groove oriented in a clockwise direction and a second helical protrusion or groove oriented in a counterclockwise direction.
Clause 237. The medical device of clause 235, wherein the elongated member is tapered near a distal end of the elongated member.
Clause 238. The medical device of clause 235, wherein the elongated member includes at least one cavity that extends radially outward from the elongated member to the outer surface.
Clause 239. The medical device of clause 238, wherein the at least one cavity has a depth that is less than a thickness of the elongated member of the elongated member.
Clause 240. The medical device of clause 238, wherein the at least one cavity is disposed at a region along the outer surface formed by intersections of the least one helical protrusion or groove.
Clause 241. The medical device of clause 238, wherein in a retrieval configuration, a portion of the at least one helical protrusion or groove or the at least one cavity is engaged with a clot fragment.
Clause 242. The medical device of clause 235, wherein in an unconstrained configuration, the medical device is within a catheter, and navigated distally along a vessel to a region proximate to a clot.
Clause 243. The medical device of clause 235, further comprising a plurality of cavities on the outer surface of the elongated member, wherein each of the plurality of cavities has a cavity depth that is less than a thickness of the elongated member.
Clause 244. The medical device of clause 243, wherein at least one of the plurality of cavities overlaps with at least one semi-continuous protrusion or groove.
Clause 245. The medical device of clause 244, wherein the plurality of cavities does not overlap with the at least one semi-continuous protrusion or groove on the outer surface of the elongated member.
Clause 246. The medical device of clause 244, wherein the plurality of cavities is spaced apart from the at least one semi-continuous protrusion or groove on the outer surface of the elongated member.
Clause 247. The medical device of clause 244, wherein the plurality of cavities is an array of cavities spaced apart from one another.
Clause 248. The medical device of clause 244, wherein the plurality of protrusions or grooves forms a plurality of kite-shaped regions on the outer surface of the elongated member, and wherein each of the plurality of cavities are located within the plurality of kite-shaped regions.
Clause 249. The medical device of clause 248, wherein the plurality of cavities is substantially centered within the plurality of kite-shaped regions.
Clause 250. A medical device, comprising: a balloon catheter having a proximal region, a distal region, and an internal lumen therebetween; an inflatable balloon coupled to the distal region of the balloon catheter, wherein the inflatable balloon is in fluid communication with an inflation port at the proximal region of the balloon catheter via an inflation channel within the balloon catheter; and a plurality of bristles distributed along the inflatable balloon or a spring shaft located, at least in part, along an axial length of the balloon catheter that includes the inflatable balloon.
Clause 251. The medical device of clause 250, wherein the distal region of the balloon catheter further comprises an intermediate layer, wherein the plurality of bristles is coupled to the intermediate layer, and the inflatable balloon is configured to change a radius of the intermediate layer from an axial center of the balloon catheter.
Clause 252. The medical device of clause 251, wherein the intermediate layer comprises a flexible material that remains coupled to the inflatable balloon when the inflatable balloon expands from a deflated configuration to an inflated configuration, wherein the radius is greater in the inflated configuration than in the deflated configuration.
Clause 253. The medical device of clause 250, wherein each bristle of the plurality of bristles comprise hook-shaped bristles, wherein each of the hook-shaped bristles has a curved distal bristle tip.
Clause 254. The medical device of clause 253, wherein each of the curved distal bristle tip of the hook-shaped bristles face, at least in part, in a proximal direction.
Clause 255. The medical device of clause 250, wherein the distal region of the balloon catheter further comprises the spring shaft located, at least in part, along the axial length of the balloon catheter that includes the inflatable balloon.
Clause 256. The medical device of clause 255, wherein the spring shaft comprises at least one coil.
Clause 257. The medical device of clause 255, wherein the spring shaft is coupled to the inflatable balloon at two or more axial locations along the balloon catheter.
Clause 258. The medical device of clause 257, where in an initial configuration the inflatable balloon is deflated, and where in a material-engagement configuration the inflatable balloon is inflated and the plurality of bristles are engaged with a material.
Clause 259. The medical device of clause 258, where in the material-engagement configuration, the spring shaft is in a flexed configuration.
Clause 260. The medical device of clause 258, wherein the material is a blood clot within a blood vessel.
Clause 261. A method of removing a material from a blood vessel, comprising: navigating a catheter into the blood vessel to a target site that is distal to the material, wherein the catheter comprises: a pusher with a distal end having an elongated member, a plurality of bristles, wherein each bristle has a proximal bristle end coupled to the elongated member and a distal bristle tip not coupled to the elongated member, and wherein the plurality of bristles comprises: a first set of bristles coupled to a first longitudinal portion of the elongated member, and a second set of bristles coupled to a second longitudinal portion of the elongated member; and wherein the elongated member has a first bristle-free region located between the first longitudinal portion and the second longitudinal portion of the elongated member; engaging the plurality of bristles with the material; retracting the catheter, the plurality of bristles, and the material in a proximal direction.
Clause 262. A method of removing a material from a blood vessel, comprising: navigating a catheter into the blood vessel to a target site that is distal to the material, wherein the catheter comprises: a pusher with a distal end having an elongated member, a plurality of bristles, wherein each bristle has a proximal bristle end coupled to the elongated member and a distal bristle tip not coupled to the elongated member, and wherein the plurality of bristles comprises: a first set of bristles coupled to a first cross-sectional portion of the elongated member, a second set of bristles coupled to a second cross-sectional portion of the elongated member; and wherein the elongated member has a first bristle-free region located between the first cross-sectional portion and the second cross-sectional portion of the elongated member; engaging the plurality of bristles with the material; retracting the catheter, the plurality of bristles, and the material in a proximal direction.
Clause 263. A method of removing a material from a blood vessel, comprising: navigating a balloon catheter into the blood vessel to a target site that is distal to the material, wherein the balloon catheter comprises an inflatable balloon having an outer surface with a plurality of bristles; inflating the balloon; engaging the plurality of bristles with the material; retracting the balloon catheter, the plurality of bristles, and the material in a proximal direction.
Clause 264. The method of clause 263, wherein the inflatable balloon is inflated for at least a portion of the retracting.
Clause 265. The method of clause 263, wherein the inflatable balloon catheter is retracted towards a distal tip of a catheter.
Clause 266. The method of clause 265, wherein the catheter is an aspiration catheter.
Clause 267. The method of clause 263, wherein the inflatable balloon in an inflated state has an outer diameter that is substantially similar to an inner diameter of the blood vessel at the target site.
Clause 268. The method of clause 263, wherein each bristle of the plurality of bristles comprises at least one hook-shaped bristle, wherein each of the at least one hook-shaped bristle has a curved distal bristle tip.
Clause 269. The method of clause 268, wherein each of the curved distal bristle tip of the hook-shaped bristles face, at least in part, in the proximal direction.
Clause 270. The method of clause 268, wherein the curved distal bristle tip of each of the at least one hook-shaped bristle has a curvature of at least 90 degrees.
Clause 271. The method of clause 268, wherein the curved distal bristle tip of each of the at least one hook-shaped bristle has a curvature of at least 135 degrees.
Clause 272. The method of clause 268, wherein the curved distal bristle tip of each of the at least one hook-shaped bristle has a curvature of at least 180 degrees.
Clause 273. The method of clause 268, wherein the curved distal bristle tip of each of the at least one hook-shaped bristle has a helical shape.
Clause 274. The method of clause 263, wherein the plurality of bristles comprises bristles of a circular shape, a square shape, a rectangular shape, a polygon shape, a triangular shape, an oval shape, a trapezoid shape, a straight line longitudinally, a zigzag line longitudinally, or a bent line longitudinally.
Clause 275. A medical device, comprising: a pusher with a distal end having an elongated member; and a set of bristle means for capturing a foreign body and coupled to the elongated member.
Clause 276. A medical device, comprising: a pusher with a distal end having an elongated member; and a set of cavity means or a set of semi-continuous protrusion or groove means for capturing a foreign body and coupled to the elongated member.
Clause 277. A medical device, comprising: a pusher with a distal end having an elongated member; and at least one capturing means for capturing a foreign body and distributed along an inflatable means associated with the elongated member.

What is claimed is:

1. A medical device, comprising:
a pusher; and
a clot-engaging device coupled to the pusher, the clot-engaging device including a plurality of bristles extending from a surface of the clot-engaging device.

2. The medical device of claim 1, wherein each bristle of the plurality of bristles comprises a first end attached to the surface of the clot-engaging device.

3. The medical device of claim 1, wherein the plurality of bristles comprises a first set of bristles and a second set of bristles, the second set of bristles being different than the first set of bristles.

4. The medical device of claim 3, wherein the first set of bristles is attached to a first cross-sectional portion of the surface of the clot-engaging device, and the second set of bristles is attached to a second cross-sectional portion of the surface of the clot-engaging device, the first cross-sectional portion and the second cross-sectional portion being separated by a bristle-free region on the surface of the clot-engaging device.

5. The medical device of claim 4, wherein the first set of bristles or the second set of bristles comprises hook-shaped bristles, each of the hook-shaped bristles having a curved distal bristle tip, the curved distal bristle tip pointing to one of a distal end of the clot-engaging device or a non-distal end of the clot-engaging device.

6. The medical device of claim 5, wherein at least a portion of a body of each bristle of the plurality of bristles extends linearly in a radial direction from a center of the clot-engaging device.

7. The medical device of claim 1, wherein each bristle of the plurality of bristles further comprises a tip shape that is one of rounded, circular, square, rectangular, polygonal, triangular, helical, oval, or trapezoidal.

8. The medical device of claim 1, wherein at least one bristle of the plurality of bristles is pointing in a tangential direction away from the surface of the clot-engaging device.

9. The medical device of claim 1, wherein the clot-engaging device is conically shaped.

10. The medical device of claim 1, wherein the plurality of bristles extends from the surface of the clot-engaging device at a radius from an axial center of the clot-engaging device, the clot-engaging device further comprises an inflatable balloon coupled to the pusher, and the inflatable balloon is configured to change the radius of the plurality of bristles from the axial center of the clot-engaging device.

11. The medical device of claim 10, wherein the clot-engaging device further comprises an intermediate layer, wherein the plurality of bristles is coupled to the intermediate layer, and the inflatable balloon is configured to change the radius of the intermediate layer from the axial center of the clot-engaging device.

12. The medical device of claim 10, further comprising a spring shaft located, at least in part, along an axial length of the inflatable balloon.

13. The medical device of claim 1, wherein the clot-engaging device further comprises at least one coil located in a coil region of the clot-engaging device between a first bristle region and a second bristle region.

14. A medical device, comprising:
a pusher; and
a clot-engaging device coupled to the pusher, the clot-engaging device including at least one semi-continuous protrusion or groove or at least one cavity on an outer surface or an inner surface of the clot-engaging device.

15. The medical device of claim 14, wherein each cavity of the at least one cavity has a cavity depth that is less than a thickness of the clot-engaging device.

16. The medical device of claim 14, wherein the at least one semi-continuous protrusion or groove has a helical pattern.

17. The medical device of claim 14, wherein a depth of the at least one semi-continuous protrusion or groove is a variable depth.

18. The medical device of claim 14, wherein the clot-engaging device is conically shaped.

19. The medical device of claim 14, wherein the at least one cavity is distributed along the outer surface of the clot-engaging device at a radius from an axial center of the clot-engaging device, the clot-engaging device further comprises an inflatable balloon coupled to the pusher, and the inflatable balloon is configured to change the radius of the at least one cavity from the axial center of the clot-engaging device.

20. A medical device, comprising:
a pusher with a distal end having an elongated member; and
a set of bristle means for capturing a foreign body and coupled to the elongated member.
